SEQUENCE LISTING (4 sequences)

SEQ ID NO: 1 - DNA, 10 bp, Artificial
  synthetic construct
ctcttggggg

SEQ ID NO: 2 - DNA, 10 bp, Artificial
  synthetic construct
gggggttctc

SEQ ID NO: 3 - DNA, 22 bp, Artificial
  synthetic construct
tcaacatcagtctgataagcta

SEQ ID NO: 4 - DNA, 27 bp, Artificial
  synthetic construct
tagcttatcagactgatgttgaggggg

(19) United States
(12) Patent Application Publication
(10) Pub. No.: US 2024/0285525 A1
(43) Pub. Date: Aug. 29, 2024

(54) METHODS AND MATERIALS FOR TREATING CANCER

(71) Applicant: The Johns Hopkins University, Baltimore, MD (US)

(72) Inventors: Efrosini Kokkoli, Baltimore, MD (US); Zachary Schneiderman, Baltimore, MD (US)

(21) Appl. No.: 18/569,366

(22) PCT Filed: Jun. 23, 2022
(86) PCT No.: PCT/US2022/034697

§ 371 Date: Dec. 12, 2023

Related U.S. Application Data
(60) Provisional application No. 63/214,056, filed on Jun. 23, 2021.

(51) Int. Cl. A61K 9/00; A61K 31/704; A61K 47/36

(52) U.S. Cl. CPC A61K 9/0092; A61K 31/704; A61K 47/36

(57) ABSTRACT

This document relates to methods and materials for treating cancer. For example, nanostructures (e.g., nanotubes) having one or more anti-cancer agents intercalated in the nanostructures are provided. In some cases, nanostructures (e.g., nanotubes) having one or more anti-cancer agents intercalated in the nanostructures can be administered to a mammal (e.g., a human) having cancer to treat the mammal.

### CROSS-REFERENCE TO RELATED APPLICATIONS

This application claims the benefit of U.S. Patent Application Ser. No. 63/214,056, filed on Jun. 23, 2021. The disclosure of the prior application is considered part of (and is incorporated by reference in) the disclosure of this application.

### TECHNICAL FIELD

This document relates to methods and materials for treating cancer. For example, this document provides nanostructures (e.g., nanotubes) including one or more anti-cancer agents. In some cases, nanostructures (e.g., nanotubes) including one or more anti-cancer agents can be administered to a mammal (e.g., a human) having cancer to treat the mammal.

### BACKGROUND INFORMATION

Glioblastoma (GBM) is the most malignant and aggressive type of primary brain tumor. Despite treatment through neurosurgery, radiation, and chemotherapy, long-term survival remains low with a high rate of recurrence, a median survival of 12-15 months and only 5.5% of patients are estimated to be alive 5 years after diagnosis (Cantrell et al., Mayo Clin. Proc., 94:1278-1286 (2019)).

### SUMMARY

A need exists for the development of novel therapies for the treatment of GBM. However, there are major hurdles in the development of therapeutics, such as their inability to cross the blood-brain barrier (BBB). Even though the BBB is disrupted during tumor progression in high-grade gliomas (known as the blood-brain tumor barrier, BBTB), its permeability is heterogeneous in GBM (Arvanitis et al., Nat. Rev. Cancer, 20:26-41 (2020); and Sarkaria et al., Neuro-Oncol., 20:184-191 (2018)).

This document provides methods and materials for treating cancer (e.g., a central nervous system (CNS) cancer such as GBM or a triple negative breast cancer (TNBC)). For example, this document provides nanostructures (e.g., nanotubes) including one or more anti-cancer agents (e.g., one or more chemotherapeutic agents) where the nanostructures are assembled from nucleic acid (NA)-amphiphiles that contain a hydrophilic NA headgroup and hydrophobic dialkyl tail, and where one or more anti-cancer agents are intercalated in and/or encapsulated within the nanostructure. In some cases, nanostructures (e.g., nanotubes) including one or more anti-cancer agents can be administered to a mammal (e.g., a human) having cancer to treat the mammal.

As demonstrated herein, nanotubes assembled from NA-amphiphiles including one or more anti-cancer agents such that the nanotubes have the anti-cancer agents intercalated in the nanotubes can bind to and internalize into cancer cells, but not healthy cells. For example, such nanotubes can cross the blood brain barrier (BBB), bind to and internalize into glioma cells and macrophages (e.g., microglia), and can accumulate in the tumoral brain hemisphere.

Having the ability to transport one or more anti-cancer agents (e.g., one or more chemotherapeutic agents) to cancer cells but not healthy cells (e.g., by administering nanostructures such as nanotubes including one or more anti-cancer agents as described herein) provides a targeted treatment for mammals having cancer. For example, using nanostructures provided herein (e.g., nanotubes including one or more anti-cancer agents) to deliver one or more anti-cancer agents to a mammal having a CNS cancer or a TNBC can specifically target cancer cells within the mammal and can reduce or eliminate toxic effects in healthy organs. Further, having the ability to transport one or more anti-cancer agents across the BBB as described herein (e.g., by administering nanostructures such as nanotubes including one or more anti-cancer agents as described herein) provides a unique and unrealized opportunity to safely and effectively treat mammals having a CNS cancer (e.g., GBM). For example, using nanostructures provided herein (e.g., nanotubes including one or more anti-cancer agents) to deliver one or more anti-cancer agents to a mammal having a CNS cancer can specifically target cancer cells within the CNS of the mammal.

In general, one aspect of this document features nanotubes comprising a chemotherapeutic agent, where the nanotube includes NA-amphiphiles, each NA amphiphile including a hydrophilic NA headgroup and hydrophobic dialkyl tail having a hydrophobic spacer, where the chemotherapeutic agent is intercalated in the nanotube, and where the chemotherapeutic agent is doxorubicin, gemcitabine, 5FU, carboplatin, cyclophosphamide, cisplatin, or oxaliplatin. The hydrophilic NA headgroup can include from about 4 nucleotides to about 52 nucleotides. The hydrophilic NA headgroup can include single stranded nucleic acid. The hydrophilic NA headgroup can include double stranded nucleic acid. The hydrophilic NA headgroup can include a non-targeting nucleotide sequence. The non-targeting nucleotide sequence can include a nucleotide sequence selected from the group consisting of CTCTTGGGGG (SEQ ID NO:1) and GGGGGTTCTC (SEQ ID NO:2). The hydrophobic dialkyl tail having the hydrophobic spacer can include a structure set forth in Formula I:

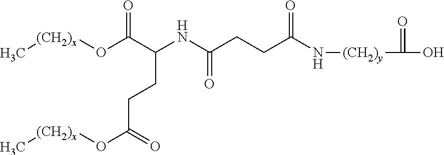

where x=15, and wherein y=11. The NA-amphiphiles can include a linker between the hydrophilic NA headgroup and the hydrophobic dialkyl tail. The linker can bea near-infrared (NIR) light sensitive linker, a pH sensitive linker, a disulfide linker, an acetal, or a positively charged polypeptide.

In another aspect, this document features nanotubes comprising a hydrophobic therapeutic agent, where the nanotube includes NA-amphiphiles, each NA amphiphile including a hydrophilic NA headgroup and hydrophobic dialkyl tail having a hydrophobic spacer, where the hydrophobic therapeutic agent is intercalated in the nanotube, and where the hydrophobic therapeutic agent is a senotherapeutic agent. The senotherapeutic agent can be ABT-263, ABT-199, A1155463, A1331852, dasatinib, quercetin, or methadone. The hydrophilic NA headgroup can include from about 4 nucleotides to about 52 nucleotides. The hydrophilic NA headgroup can include single stranded nucleic acid. The hydrophilic NA headgroup can include double stranded nucleic acid. The hydrophilic NA headgroup can include a non-targeting nucleotide sequence. The non-targeting nucleotide sequence can include a nucleotide sequence selected from the group consisting of CTCTTGGGGG (SEQ ID NO:1) and GGGGGTTCTC (SEQ ID NO:2). The hydrophobic dialkyl tail having the hydrophobic spacer can include a structure set forth in Formula I:

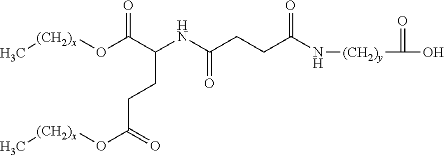

where x=15, and wherein y=11. The NA-amphiphiles can include a linker between the hydrophilic NA headgroup and the hydrophobic dialkyl tail. The linker can bea near-infrared (NIR) light sensitive linker, a pH sensitive linker, a disulfide linker, an acetal, or a positively charged polypeptide.

In another aspect, this document features nanotubes comprising a chemotherapeutic agent, where the nanotube includes NA-amphiphiles, each NA amphiphile including a hydrophilic NA headgroup and hydrophobic dialkyl tail having a hydrophobic spacer, where the chemotherapeutic agent is encapsulated within the nanotube, and where the chemotherapeutic agent is tamoxifen, paclitaxel, docetaxel, temozolomide, camptothecin, curcumin, dexamethasone, furosemide, IPI-549, and KPT-9274. The hydrophilic NA headgroup can include from about 4 nucleotides to about 52 nucleotides. The hydrophilic NA headgroup can include single stranded nucleic acid. The hydrophilic NA headgroup can include double stranded nucleic acid. The hydrophilic NA headgroup can include a non-targeting nucleotide sequence. The non-targeting nucleotide sequence can include a nucleotide sequence selected from the group consisting of CTCTTGGGGG (SEQ ID NO:1) and GGGGGTTCTC (SEQ ID NO:2). The hydrophobic dialkyl tail having the hydrophobic spacer can include a structure set forth in Formula I:

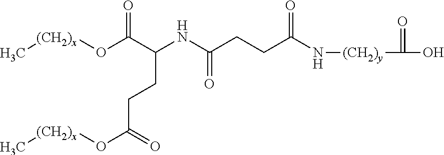

where x=15, and wherein y=11. The NA-amphiphiles can include a linker between the hydrophilic NA headgroup and the hydrophobic dialkyl tail. The linker can bea near-infrared (NIR) light sensitive linker, a pH sensitive linker, a disulfide linker, an acetal, or a positively charged polypeptide.

In another aspect, this document features nanotubes comprising NA-amphiphiles, where each NA-amphiphile includes a hydrophilic NA headgroup and hydrophobic dialkyl tail having a hydrophobic spacer, where the hydrophilic NA headgroup includes a microRNA (miRNA) or a miRNA mimic. The miRNA can be miR-34a, miR128, miR-21, miR-603, miR-218, miR-219, miR-183m, miR-451, miR-133, miR-134, miR-302c, miR-324, miR-379, miR-491, miR-340, miR-7, miR-128, miR-368-3p, miR-10b, miR-15a, miR-16, miR-17-5p, miR-26a, miR-29, miR-29b, miR-31, miR-33a, miR-34, miR-93, miR-101, miR-101-3p, miR-122, miR-122a, miR-125b, miR-130a, miR-133-b, miR-136, miR-143, miR-145, miR-146a-5p, miR-148a, miR-181d, miR-182, miR-183, miR-195, miR-199a-5p, microRNAs in the miR-200 family, miR-203, miR-203b-3p, miR-205, miR-206, miR-217, miR-298, miR-375, miR-490-3p, miR-539, miR-542-3p, miR-613, miR-638, miR-940, or a microRNAin the let-7 family. The hydrophobic dialkyl tail having the hydrophobic spacer can include a structure set forth in Formula I.

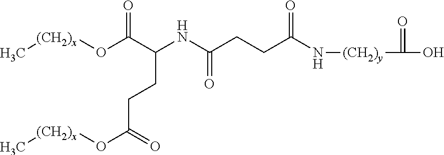

where x=15, and wherein y=11. The NA-amphiphiles can include a linker between the miRNA or the miRNA mimic and the hydrophobic dialkyl tail. The linker can be a NIR light sensitive linker, a pH sensitive linker, a disulfide linker, an acetal, or a positively charged polypeptide.

In another aspect, this document features nanotubes comprising NA-amphiphiles, where each NA-amphiphile includes a hydrophilic NA headgroup and hydrophobic dialkyl tail having a hydrophobic spacer, where the hydrophilic NA headgroup includes an anti-miRNA. The anti-mRNA can be anti-miR-9, anti-miR-10b, anti-miR-20a-5p, anti-miR-21, anti-miR-25-3p, anti-miR-26a, anti-miR-27a, anti-miR-29b, anti-miR-30b/30e/30d, anti-miR-31, anti-miR-34a, anti-miR-103, anti-miR-107, anti-miR-122, anti-miR-125b, anti-miR-126, anti-miR-139, anti-miR-143, anti-miR-146a, anti-miR-146b-5p, anti-miR-153, anti-miR-155, anti-miR-181a/b, anti-miR-182, anti-miR-199a-3p, anti-miR-199a-5p, anti-miR-200s, anti-miR-210, anti-miR-214, anti-miR-221, anti-miR-222, anti-miR-223, anti-miR-335, anti-miR-342, anti-miR-373, anti-miR-455-3p, anti-miR-429, anti-miR-493, anti-miR-494, anti-miR-520c, anti-miR-520h, or anti-miR-1908, The hydrophobic dialkyl tail having the hydrophobic spacer can include a structure set forth in Formula I:

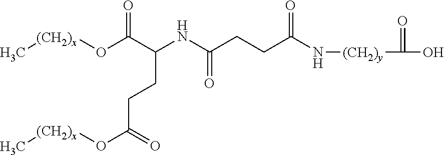

where x=15, and wherein y=11. The NA-amphiphiles can include a linker between the anti-miRNA and the hydrophobic dialkyl tail. The linker can be a NIR light sensitive linker, a pH sensitive linker, a disulfide linker, an acetal, or a positively charged polypeptide.

In another aspect, this document features nanotubes comprising NA-amphiphiles, where each NA-amphiphile includes a hydrophilic NA headgroup and hydrophobic dialkyl tail having a hydrophobic spacer, where the hydrophilic NA headgroup includes a small interfering RNA (siRNA). The hydrophobic dialkyl tail having the hydrophobic spacer can include a structure set forth in Formula I:

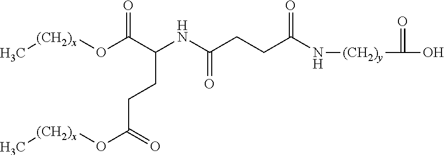

where x=15, and wherein y=11. The NA-amphiphiles can include a linker between the siRNA and the hydrophobic dialkyl tail. The linker can be a NIR light sensitive linker, a pH sensitive linker, a disulfide linker, an acetal, or a positively charged polypeptide.

In another aspect, this document features methods for treating a mammal having cancer. The methods can include, or consist essentially of, administering a composition comprising any one or more of the nanotubes described herein to a mammal having cancer.

The mammal can be a human. The cancer can be a glioblastoma, an astrocytoma, an oligodendroglioma, an oligoastrocytoma, an ependymoma, a medulloblastoma, a meningioma, a diffuse intrinsic pontine glioma (DIPG), a breast cancer, a colon cancer, a liver cancer, a pancreatic cancer, a prostate cancer, a lung cancer, an ovarian cancer, a kidney cancer, a spleen cancer, or a gastric cancer.

In another aspect, this document features methods for repolarizing a tumor-associated microglia and macrophage (TAM) to an M1-phenotype within a mammal having cancer. The methods can include, or consist essentially of, administering a composition comprising any one or more of the nanotubes described herein to a mammal having cancer. The mammal can be a human. The cancer can be a glioblastoma, an astrocytoma, an oligodendroglioma, an oligoastrocytoma, an ependymoma, a medulloblastoma, a meningioma, a diffuse intrinsic pontine glioma (DIPG), a breast cancer, a colon cancer, a liver cancer, a pancreatic cancer, a prostate cancer, a lung cancer, an ovarian cancer, a kidney cancer, a spleen cancer, or a gastric cancer.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1C. 10 nucleotide (nt) ssDNA-amphiphiles form parallel G-quadruplexes and self-assemble into hollow nanotubes. FIG. 1A) Chemical structures of the 10 nt ssDNA-amphiphiles. FIG. 1B) CD spectra in Milli-Q water and PBS of the free 10 nt ssDNA and the ssDNA-amphiphiles. FIG. 1C) Schematic representation of the self-assembled nanotubes (top) and cryo-TEM image (bottom) of the ssDNA nanotubes. The white arrows point to nanotubes that are viewed end-on, demonstrating their hollow nature.

FIGS. 2A-2D. Preferential uptake of nanotubes by GL261 GBM cells via the macropinocytosis pathway and scavenger receptors. FIG. 2A) Confocal microscopy of nanotubes (shown in green) after incubation with GL261 GBM cells and C8-D1A normal astrocytes for 24 hours at 37° C. Nuclei are shown in gray and cell membranes in red. Scale bars are 20 μm. FIG. 2B) Confocal images of nanotubes (shown in red) after incubation with GL261 cells for 24 hours at 37° C. Frames show slices at 2, 3, 4, and 5 μm above the glass coverslip. Nuclei are shown in gray, early endosomes in blue and acidic organelles in green. Colocalization of nanotubes with early nanotubes is shown in magenta and with acidic organelles in yellow/orange. Scale bars are 20 μm. FIG. 2C) Manders coefficient values quantify the different levels of colocalization between nanotubes and early endosomes, acidic organelles and other vesicles. FIG. 2D) GL261 association of nanotubes in the presence of different endocytosis inhibitors. Data are shown as mean±SD (n=3). Statistical significance with that of the control (incubation with nanotubes in the absence of inhibitors) was determined using a two-sided unpaired t-test; * P<0.05, ** P<0.01, *** P<0.005. There was no significant statistical difference for all other treatments (P>0.05).

FIGS. 3A-3C. Nanotube stability and tumor preferential uptake. FIG. 2A) Stability of nanotubes in different concentrations of serum, DNase I and exonuclease III.

FIG. 3B) Schematic of bilateral intracranial injections of nanotubes to mice with GL261 tumor only on the right hemisphere of their brain. NIR fluorescent images of mouse brains excised at different time points: mouse 1 at 45 minutes, mouse 2 at 70 minutes, mouse 3 at 105 minutes. Control mouse had a GL261 tumor but did not receive intracranial nanotube injections. The radiant efficiency of NIR fluorescently-labeled nanotubes is shown with a heat map. Wide-field fluorescent microscopy image of a brain tissue section excised from mouse 2, with nuclei stained in blue, glial fibrillary acidic protein stained in green, NIR fluorescently-labelled nanotubes shown in red. Scale bar is 500 μm. FIG. 2C) Mice bearing orthotopic tumors of GL261 cells expressing GFP were intracranially injected with nanotubes. Mice were sacrificed 3 hours post nanotube injection, and tumor tissues were removed and processed. Maximum intensity projection of a confocal microscopy image showing colocalization of GL261 cells (shown in green) with the nanotubes (shown in red). Scale bars are 20 μm.

FIGS. 4A-4F. Local brain delivery of DOX and nanotubes intercalating DOX. FIG. 4A) Preparation and treatment schedule of mice. The right side of the brain was injected with 10⁴ GL261-Luc cells on day 0. Immediately after that, a micro-osmotic Alzet pump was implanted subcutaneously and the cannula, connected to the pump through a catheter, was lowered into the same burr hole used to inject the cells. The pumps were loaded with either PBS, 70 μM of DOX (0.2 mg DOX/Kg mouse), nanotubes (NT) at 95 μM of ssDNA-amphiphiles or DOX intercalated in the nanotubes (NT-DOX) at the same concentrations of DOX and amphiphiles and delivered their content in about 14 days at a pumping rate of 0.25 μL/hour. FIG. 4B) Representative bioluminescence images of mice at different time points. Scale bars are shown on the side. FIG. 4C) Quantification of tumor bioluminescence values in the different treatment groups over time. FIG. 4D) Body weight of mice in different groups during treatment. In FIGS. 4C and 4D data are shown as mean±SEM (n=9-10). For the NT group, data are not reported on day 28 as only 3 mice were alive. Statistical significance on day 28 was determined using a two-sided unpaired t-test; † P>0.05, * P<0.05. FIG. 4E) Survival curves corresponding to the different treatment groups (n=9-10). Statistical significance was determined using a two-sided log-rank test; * P<0.05. There was no significant statistical difference for pairs without brackets (P>0.05). FIG. 4F) Representative images of H&E staining of tumors and other organs from mice that received different treatments. Images were taken with 20× objective lens. Scale bars are 100 μm.

FIGS. 5A-5E. Nanotube biodistribution and BBTB crossing. FIG. 5A) Schematic of intravenous (IV) injection of nanotubes to mice with GL261 tumor on the right hemisphere of their brain. FIG. 5B) Full body volumetric 3D reconstruction of PET/CT mice imaged at 1, 3 and 24 hours after intravenous injection of ⁶⁴Cu-radiolabeled nanotubes. The PET intensity scalebar for all images has units of Ci/mL, and the intensity of the PET signal has not been adjusted for the half-life of ⁶⁴Cu. FIG. 5C) Ex vivo biodistribution of ⁶⁴Cu-labeled nanotubes at 3 or 24 hours after intravenous injection to mice bearing GL261 orthotopic tumors. Radioactivity and weights of different organs were measured, data were adjusted for the 12.7 hour half-life of ⁶⁴Cu and plotted as mean SEM (n=3-4). Statistical significance was determined using a two-sided unpaired t-test; * P<0.05. There was no significant statistical difference for pairs without brackets (P>0.05).

FIGS. 5D-5E) Mice bearing orthotopic tumors of GL261 cells expressing GFP were injected intravenously with HEX-labeled nanotubes. Mice were sacrificed 6 h post nanotube injection, and tumor tissues were removed and processed with confocal microscopy (FIG. 5D) and flow cytometry (FIG. 5E). FIG. 5D) Maximum intensity projection of a confocal microscopy image showing colocalization of GL261 cells (shown in green) with the nanotubes (shown in red). Scale bars are 20 μm. FIG. 5E) Flow cytometry plots from GL261-GFP tumors from mice that were intravenously injected with PBS or HEX-labeled nanotubes (NT IV).

FIG. 6A-6B. Synthesis schemes of ssDNA-amphiphiles. FIG. 6A) ssDNA used as purchased, with or without a HEX fluorophore at the 5′. FIG. 6B) Modifications added to the ssDNA via an alkyne reaction. The insets show the chemical structures of all modifications used.

FIG. 7. Cell viability after exposure to endocytosis inhibitors. Viability of GL261 cells treated with different endocytosis inhibitors at the same concentrations used for FIG. 2D. Data are shown as mean±SD (n=3). Statistical significance with that of the control was determined using a two-sided unpaired t-test; P>0.05 for all inhibitors.

FIG. 8. Nanotube retention by tumor hemisphere. Mouse 1 (FIG. 3B) bearing an orthotopic GL261 tumor on the right hemisphere received bilateral intracranial injections of NIR fluorescently-labelled nanotubes. Wide-field fluorescent microscopy images of brain tissue sections excised from mouse 1, showing the nanotubes (shown in red) only on the right, tumor-bearing hemisphere. The white arrow shows an area close to the left hemisphere injection point. Scale bars are 500 μm.

FIG. 9. Nanotube colocalization with tumor associated microglia and macrophages (TAMs). Mice bearing orthotopic GL261 tumors received intracranial injections of nanotubes. Mice were sacrificed 3 hours post intracranial injection, and tumor tissues were removed and processed. Confocal microscopy images showing colocalization of nanotubes (shown in red) with TAMs (shown in yellow). Nuclei were stained with DAPI (blue). Scale bars are 20 μm.

FIG. 10. DOX release from nanotubes. Release profile of DOX from nanotubes in PBS at 37° C. Results are reported as mean±SD (n=3).

FIG. 11. GL261 viability after exposure to different treatments. Viability of GL261 cells treated with ssDNA nanotubes (NT) at 5-6.4 μM of ssDNA-amphiphiles, free DOX at 5 μg/mL, or DOX intercalated in the ssDNA nanotubes (NT-DOX) at the same DOX and amphiphile concentrations. Cells were incubated with the different samples for 12 hours at 37° C., washed, replenished with media and incubated for another 36 hours at 37° C. Data are presented as mean±SD (n=6). Statistical analysis was performed by one-way ANOVA with Tukey's honest significant difference post-hoc test; † P>0.05, * * * P<0.005, for all other groups P<0.00001.

FIGS. 12A-12B. Hematoxylin and eosin (H&E) staining of brains. Representative images of H&E staining of brain tissues from mice that received different treatments (PBS, NT, DOX, NT-DOX) and either survived at the end of the experiment (FIG. 12; day 82 from day of surgery) or died during the experiment (FIG. 12B). Images were taken with 1× objective lens.

FIGS. 13A-13B. Nanotube accumulation in the brain after intravenous injection. FIG. 13A) Tail-view maximum intensity projection of PET/CT scans of mice heads at 1, 3 and 24 hours after intravenous injection of ⁶⁴Cu-radiolabeled nanotubes to mice bearing GL261 orthotopic tumors. The intensity of the PET signal was not adjusted for the half-life of ⁶⁴Cu. FIG. 13B) Percent of maximum PET brain signal as a function of distance from the left side of the brain from the head-view images at 1 hour. Background signal was subtracted, and radiation intensity values were not adjusted for the half-life decay of ⁶⁴Cu.

FIGS. 14A-14B. FIGS. 14A) Confocal microscopy images of nanotubes incubated for 3 hours at 37° C. with Hs578Bst normal breast cells, MCF-7 breast cancer cells that express estrogen receptors and the following triple negative breast cancer (TNBC) cells: BT549, SUM159 and MDA-MB-231. Nanoparticles are shown in green, nuclei in gray and cell membranes in red. Scale bars are 20 m. FIGS. 14B) Flow cytometry results after incubating TNBC cells (SUM159, BT549 and MDA-MB-231) with nanotubes for 3, 12, or 24 hours at 37° C. The cell autofluorescence was subtracted from all samples. Data are presented as the mean±SD (n=3). One-way ANOVA with Tukey's HSD post-hoc analysis was used to determine statistical significance; for each cell and for all time pairs P<0.05.

FIG. 15. Viability of TNBC cells after treatment with empty nanotubes (NT) at 1.15 μM of ssDNA-amphiphiles for SUM159 and BT549 or 11.1 μM for MDA-MB-231, free DOX at 0.5 μg/mL for SUM159 and BT549 or 5 μg/ml for MDA-MB-231, or DOX intercalated in the ssDNA nanotubes (NT-DOX) at the same DOX and amphiphile concentrations. Cells were incubated with the different samples for 12 hours at 37° C., washed, replenished with media and incubated for another 36 hours at 37° C. Data are shown as percentage of untreated cells and presented as mean±SD (n=3). One-way ANOVA with Tukey's HSD post-hoc analysis was used to determine statistical significance; t p>0.05, for all other groups P<0.00001.

FIGS. 16. Confocal microscopy images of nanotubes incubated for 3 hours at 37° C. with CT26 colon cancer cells, HepG2 liver cancer cells and PANC-1 pancreatic cancer cells. Nanoparticles are shown in green, nuclei in gray and cell membranes in red. Scale bars are 20 m.

FIGS. 17A-17B. FIGS. 17A) Confocal microscopy images of anti-miR-21 nanotubes incubated with A172 GBM cells for 3 hours at 37° C. Nanoparticles are shown in green, nuclei in blue and cell membranes in red. Scale bar is 20 m. FIGS. 17B) Viability of A172 cells after treatment with free DOX (0.2 μg/mL), anti-miR-21 nanotubes (90 nM), or anti-miR-21 nanotubes followed by DOX at the same concentrations. Data are shown as percentage of untreated cells and presented as mean±SD (n=5). One-way ANOVA with Tukey's HSD post-hoc analysis was used to determine statistical significance; p<0.001 for all pairs.

FIGS. 18A-18B. FIGS. 18A) Fluorescent microscopy images of miR21 duplex nanotubes formed after hybridization. FIGS. 18B) Confocal microscopy image of miR-21 nanotubes incubated with A172 GBM cells for 3 hours at 37° C. Nanoparticles are shown in green, nuclei in blue and cell membranes in red. Scale bar is 20 m.

FIGS. 19. Schematic representation of a dynamic (peptide-NA)-amphiphile that composes the nanotubes, where the peptide-NA release from the amphiphile and nanotube after a trigger. The nucleic acid (NA) can be single-stranded or double-stranded. Not drawn to scale.

FIGS. 20A-20C. Cryo-TEM images of ssDNA-amphiphiles that self-assembled into small micelles and short nanotubes (FIG. 20A), micron-long nanotubes formed from ssDNA-amphiphiles via the excess tail method (FIG. 20B), and short nanotubes prepared from long nanotubes after probe sonication (FIG. 20C). White arrows highlight tubes viewed head on which demonstrate the hollow nature of the nanotubes. Scale bars, 200 nm.

FIGS. 21A-21C. FIG. 21A) Cytotoxicity of ABT-263 encapsulated in ssDNA nanotubes to proliferating or senescent MDA-MB-231 TNBC cells for 48 hours at 37° C. Data are shown as means±SD (n=3). Statistical significance was assessed between the proliferating and senescent groups for each concentration using pairwise t-tests; *P<0.05. All other groups were not statistically significant (P>0.05). Free doxorubicin (DOX), free DOX+ABT-263 encapsulated in nanotubes (DOX+ABT-263-NT), DOX intercalated in the nanotubes (DOX-NT), and DOX intercalated in the nanotubes and ABT-263 encapsulated in the nanotubes (DOX-NT+ABT-263-NT) were delivered to proliferating MDA-MB-231 cells (FIG. 21B) and to senescent MDA-MB-231 cells (FIG. 21C) for 48 hours at 37° C. to evaluate cytotoxicity. Concentration of DOX was 0.5 μg/mL, and concentration of ABT-263 was 0.1 μM. Data are shown as means±SD (n=3). Statistical significance was assessed between treatments using pairwise t-tests; * P<0.05. All other combinations were not statistically significant (P>0.05).

FIG. 22. Cytotoxicity of free KPT-9274 or KPT-9274 encapsulated in ssDNA nanotubes to U87 GBM cells after 72 hours incubation at 37° C. Data are shown as means SD (n=4). Statistical significance was assessed using pairwise t-tests; †P>0.05.

FIGS. 23A-23B. Freshly isolated primary CD14+human monocytes were first differentiated to M0 and then to M2 macrophages. The M2 macrophages were treated with either 100 nM IPI-549 or 1 μM thiostrepton (TS), free or encapsulated in the nanotubes (NT).

FIG. 23A) Cells were evaluated for surface markers CD80 and CD86 via flow cytometry (pairwise t-test; † P>0.05). FIGS. 23B) Cells were evaluated for the expression of different genes at the mRNA level via RT-qPCR.

FIG. 24. Cryo-TEM image of nanotubes formed via the LBL method with a fucoidan outer layer (NT-F).

FIGS. 25A-25C. mRNA levels of miR-21 relative to PBS control determined by RT-qPCR in U87 GBM cells (FIG. 25A), MDA-MB-231 TNBC cells (FIG. 25B), and Panc 10.05 pancreatic cancer cells (FIG. 25C), following exposure to indicated treatments for 48 hours at 37° C. Data are presented as mean±SD (n=3-4). Statistical significance was evaluated with one-way ANOVA with Tukey's HSD post-hoc analysis. Brackets show pairs that were not statistically different († P>0.05). All other pairs were statistically significant (P<0.05).

FIGS. 26A-26B. Mean squared displacement (MSD) of U87 GBM cells (FIG. 26A) and MDA-MB-231 TNBC cells (FIG. 26B) that were embedded in collagen I gel, treated for 72 hours with PBS (control), 270 nM of anti-miR-21 LBL nanotubes (NT-F and NT-10), or free anti-miR-21 complexed with RNAiMAX (RNAiMAX), and tracked for 6 hours after treatment. Data are presented as mean±SD (n=2-3). Statistical significance was evaluated with one-way ANOVA with Tukey's HSD post-hoc analysis. Symbols directly above each bar represent the significance compared to the control: * P<0.05; ** P<0.005; † P>0.05. There was no significant statistical difference between all other pairs (P>0.05).

FIG. 27. Freshly isolated primary CD14+human monocytes were first differentiated to M0 and then to M2 macrophages. The M2 macrophages were treated with 180 nM of LBL anti-miR-21 NT-F nanotubes. Cells evaluated for the expression of different genes at the mRNA level via RT-qPCR.

### DETAILED DESCRIPTION

This document provides methods and materials for treating cancer (e.g., a CNS cancer such as GBM or a TNBC). For example, this document provides nanostructures (e.g., nanotubes) including one or more anti-cancer agents (e.g., one or more chemotherapeutic agents) where the nanostructures are assembled from NA-amphiphiles that contain a hydrophilic NA headgroup and hydrophobic dialkyl tail, and have one or more anti-cancer agents (e.g., one or more hydrophilic anti-cancer agents) intercalated within the NA-amphiphiles. In some cases, nanostructures (e.g., nanotubes) including one or more anti-cancer agents intercalated in the nanostructures can be administered to a mammal (e.g., a human) having cancer to treat the mammal. For example, this document provides nanostructures (e.g., nanotubes) including one or more anti-cancer agents (e.g., one or more chemotherapeutic agents) where the nanostructures are assembled from NA-amphiphiles that contain a hydrophilic NA headgroup and hydrophobic dialkyl tail, and have one or more anti-cancer agents (e.g., one or more hydrophobic anti-cancer agents) encapsulated within the NA-amphiphiles. In some cases, nanostructures (e.g., nanotubes) having one or more anti-cancer agents encapsulated within the nanostructures can be administered to a mammal (e.g., a human) having cancer to treat the mammal.

In some cases, a nanostructure provided herein (e.g., a nanotube including one or more anti-cancer agents) can be assembled from a plurality of identical NA-amphiphiles that contain a hydrophilic NA headgroup and hydrophobic dialkyl tail.

In some cases, a nanostructure provided herein (e.g., a nanotube including one or more anti-cancer agents) can be assembled from a population of two or more (e.g., two, three, four, five, or more) different NA-amphiphiles that each contain a hydrophilic NA headgroup and hydrophobic dialkyl tail.

A nanostructure provided herein (e.g., a nanotube including one or more anti-cancer agents) can be any appropriate type of nanostructure. Examples of nanostructures that can be assembled from NA-amphiphiles include, without limitation, nanotubes, twisted nanotapes, helical nanotapes, ribbons, micelles (e.g., cylindrical micelles, spherical micelles, and ellipsoidal micelles), toroids, and vesicles. In some cases, a nanostructure provided herein can be assembled from NA-amphiphiles that begin to twist to form a twisted nanotape, continues twisting to form a helical nanotape, and can ultimately transition into a nanotube.

A nanostructure provided herein (e.g., a nanotube including one or more anti-cancer agents) can be assembled (e.g., can self-assemble) from any appropriate NA-amphiphiles. A hydrophilic NA headgroup that can be included in a NA-amphiphile that can be used to form a nanostructure provided herein can include DNA, RNA, or a combination thereof. When a hydrophilic NA headgroup that can be included in a NA-amphiphile that can be used to form a nanostructure provided herein includes RNA, the RNA can be any type of RNA (e.g., microRNAs (miRNAs), small interfering RNA (siRNAs), miRNA mimics, and anti-miRNAs). A hydrophilic NA headgroup that can be included in a NA-amphiphile that can be used to form a nanostructure provided herein can be a single-stranded nucleic acid (ssNA) or a double-stranded nucleic acid (dsNA). A hydrophilic NA headgroup that can be included in a NA-amphiphile that can be used to form a nanostructure provided herein can be any appropriate length. In some cases, a NA headgroup that can be included in a NA-amphiphile that can be used to form a nanostructure provided herein can include from about 4 nucleotides to about 52 nucleotides (e.g., from about 4 nucleotides to about 50 nucleotides, from about 4 nucleotides to about 45 nucleotides, from about 4 nucleotides to about 40 nucleotides, from about 4 nucleotides to about 35 nucleotides, from about 4 nucleotides to about 30 nucleotides, from about 4 nucleotides to about 25 nucleotides, from about 4 nucleotides to about 20 nucleotides, from about 4 nucleotides to about 25 nucleotides, from about 4 nucleotides to about 10 nucleotides, from about 5 nucleotides to about 52 nucleotides, from about 10 nucleotides to about 52 nucleotides, from about 15 nucleotides to about 52 nucleotides, from about 20 nucleotides to about 52 nucleotides, from about 25 nucleotides to about 52 nucleotides, from about 30 nucleotides to about 52 nucleotides, from about 35 nucleotides to about 52 nucleotides, from about 40 nucleotides to about 52 nucleotides, from about 45 nucleotides to about 52 nucleotides, from about 5 nucleotides to about 50 nucleotides, from about 10 nucleotides to about 45 nucleotides, from about 15 nucleotides to about 40 nucleotides, from about 20 nucleotides to about 35 nucleotides, from about 25 nucleotides to about 30 nucleotides, from about 5 nucleotides to about 15 nucleotides, from about 10 nucleotides to about 20 nucleotides, from about 15 nucleotides to about 25 nucleotides, from about 20 nucleotides to about 30 nucleotides, from about 25 nucleotides to about 35 nucleotides, from about 30 nucleotides to about 40 nucleotides, from about 35 nucleotides to about 45 nucleotides, or from about 40 nucleotides to about 50 nucleotides). For example, a hydrophilic NA headgroup that can be included in a NA-amphiphile that can be used to form a nanostructure provided herein can be about 10 nucleotides in length. For example, a hydrophilic NA headgroup that can be included in a NA-amphiphile that can be used to form a nanostructure provided herein can be about 22 nucleotides in length. For example, a hydrophilic NA headgroup that can be included in a NA-amphiphile that can be used to form a nanostructure provided herein can be about 27 nucleotides in length. A hydrophilic NA headgroup that can be included in a NA-amphiphile that can be used to form a nanostructure provided herein can have any appropriate nucleotide sequence. In some cases, hydrophilic NA headgroup that can be included in a NA-amphiphile that can be used to form a nanostructure provided herein can have a random (e.g., a non-targeting) sequence. Examples of nucleotide sequences that can be included in hydrophilic NA headgroup that can be included in a NA-amphiphile that can be used to form a nanostructure provided herein include, without limitation, CTCTTGGGGG (SEQ ID NO:1), GGGGGTTCTC (SEQ ID NO:2), TCAACATCAGTCTGATAAGCTA (SEQ ID NO:3), and TAGCTTATCAGACTGATGTTGAGGGGG (SEQ ID NO:4). In some cases, a nucleotide sequence that can be included in hydrophilic NA headgroup that can be included in a NA-amphiphile that can be used to form a nanostructure provided herein can bind to one or more scavenger receptors.

A hydrophobic dialkyl tail that can be included in a NA-amphiphile that can be used to form a nanostructure provided herein (e.g., a nanotube including one or more anti-cancer agents) can be any appropriate hydrophobic dialkyl tail. A hydrophobic dialkyl tail of a NA-amphiphile that can be used to form a nanostructure provided herein can include alkyl chains having any appropriate length. In some cases, an alkyl chain in a dialkyl tail of a NA-amphiphile that can be used to form a nanostructure provided herein can be a hydrocarbon chain having from about C4 to about C30 (e.g., from about C4 to about C25, from about C4 to about C₂₀, from about C₄ to about C₁₅, from about C₄ to about C₁₀, from about C₅ to about C₃₀, from about C₁₀ to about C₃₀, from about C₁₅ to about C₃₀, from about C₂₀ to about C₃₀, from about C₂₅ to about C₃₀, from about C₅ to about C₂₅, from about C₁₅ to about C₂₀, from about C₅ to about C₁₀, from about C₁₀ to about C₁₅, or from about C₂₀ to about C₂₅). For example, an alkyl chain in a dialkyl tail of a NA-amphiphile that can be used to form a nanostructure provided herein can be a C₁₆ hydrocarbon chain. In some cases, each alkyl chain in a dialkyl tail of a NA-amphiphile that can be used to form a nanostructure provided herein can have the same length. In some cases, two or more alkyl chains in a dialkyl tail of a NA-amphiphile that can be used to form a nanostructure provided herein can have a different length.

In some cases, a nanostructure provided herein (e.g., a nanotube including one or more anti-cancer agents) can include a spacer (e.g., a hydrophobic spacer). For example, a nanostructure provided herein can be assembled from NA-amphiphiles that include a spacer between the hydrophilic NA headgroup and hydrophobic dialkyl tail. When a spacer is included between the hydrophilic NA headgroup and hydrophobic dialkyl tail of a NA-amphiphile that can be used to form a nanostructure provided herein, the spacer can be a hydrophobic spacer. Examples of spacers that can be included between the hydrophilic NA headgroup and hydrophobic dialkyl tail of a NA-amphiphile that can be used to form a nanostructure provided herein include, without limitation, alkyl spacers and positively charged spacers. When a spacer is an alkyl spacer, the spacer can be any appropriate alkyl spacer. An alkyl spacer can be saturated or unsaturated. An alkyl spacer can be a hydrocarbon chain having from about C₂ to about C₃₀ (e.g., from about C₂ to about C₂₅, from about C₂ to about C₂₀, from about C₂ to about C₁₅, from about C₂ to about C₁₀, from about C₅ to about C₃₀, from about C₁₀ to about C₃₀, from about C₁₅ to about C₃₀, from about C₂₀ to about C₃₀, from about C₂₅ to about C₃₀, from about C₅ to about C₂₅, from about C₁₅ to about C₂₀, from about C₅ to about C₁₀, from about C₁₀ to about C₁₅, or from about C₂₀ to about C₂₅). For example, an alkyl spacer can be a C₁₂ alkyl spacer.

In some cases, a hydrophobic dialkyl tail having a hydrophobic spacer can include a structure set forth in Formula I.

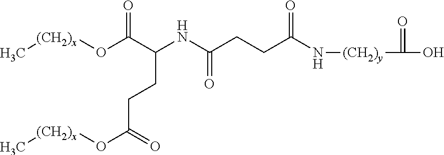

where x can be from 3 to 29, and y can be from 1 to 29. For example, a hydrophobic dialkyl tail having a hydrophobic spacer can include a structure set forth in Formula I where x is 15 and where y is 11.

In some cases, a nanostructure provided herein (e.g., a nanotube including one or more anti-cancer agents) can be assembled from NA-amphiphiles that include a linker. For example, a NA-amphiphile can include a linker between the hydrophilic NA headgroup and an anti-cancer agent. For example, a NA-amphiphile can include a linker between the hydrophilic NA headgroup and a hydrophobic tail. When a linker is included in a NA-amphiphile that can be used to form a nanostructure provided herein, the linker can be any appropriate linker. In some cases, a linker can be sensitive to a stimulus (e.g., near-infrared (NIR) light and pH) such that stimulus can be used to trigger a release of the anti-cancer agent(s) from the nanostructure. In some cases, a linker can promote escape of the nanostructure from endosomes and/or lysosomes after cell internalization. Examples of linkers that can be included between the hydrophilic NA headgroup and hydrophobic dialkyl tail of a NA-amphiphile that can be used to form a nanostructure provided herein include, without limitation, NIR light sensitive linkers (e.g., heptamethine cyanine caging group, and 1-(bromomethyl)-4,5-dimethoxy-2-nitrobenzene), pH sensitive linkers (e.g., boronic acid-catechol bonding), disulfide linkers, acetals, and positively charged polypeptides (e.g., Aureinl.2, [D]-H6L9, R4, R8, TAT). In some cases, a linker that can be included between the hydrophilic NA headgroup and hydrophobic dialkyl tail of a NA-amphiphile that can be used to form a nanostructure provided herein can be selected based on its ability to influence assembly (e.g., self-assembly) of the NA-amphiphiles into a nanostructure (e.g., a nanotube). In some cases, a linker can be as described elsewhere (see, e.g., Pearce et al., Chem. Commun., 50: 210-212 (2014); and Kuang et al., Advanced Drug Delivery Reviews, 110-111:80-101 (2017)).

A nanostructure provided herein (e.g., a nanotube including one or more anti-cancer agents) can include any appropriate one or more (e.g., one, two, three, four, or more) anti-cancer agents. An anti-cancer agent can be any appropriate type of molecule (e.g., small molecules, nucleic acids, and polypeptides such as antibodies). In some cases, an anti-cancer agent can be a chemotherapeutic agent. Examples of anti-cancer agents that can be included in a nanostructure provided herein (e.g., a nanotube including one or more anti-cancer agents) include, without limitation, doxorubicin, epirubicin, tamoxifen, paclitaxel, docetaxel, gemcitabine, 5FU, carboplatin, cyclophosphamide, temozolomide, cisplatin, IPI-549, camptothecin, curcumin, dexamethasone, furosemide, oxaliplatin, and KPT-9274. In some cases, an anti-cancer agent can be a nucleic acid (e.g., miRNAs, siRNAs, miRNA mimics, and anti-miRNAs). Examples of nucleic acids that can used as an anti-cancer agent and can be included in a nanostructure provided herein (e.g., a nanotube including one or more anti-cancer agents) include, without limitation, miR-34a, miR128, miR-21, miR-603, miR-218, miR-219, miR-183m, miR-451, miR-133, miR-134, miR-302c, miR-324, miR-379, miR-491, miR-340, miR-7, miR-128, miR-368-3p, miR-10b, miR-15a, miR-16, miR-17-5p, miR-26a, miR-29, miR-29b, miR-31, miR-33a, miR-34, miR-93, miR-101, miR-101-3p, miR-122, miR-122a, miR-125b, miR-130a, miR-133-b, miR-136, miR-143, miR-145, miR-146a-5p, miR-148a, miR-181d, miR-182, miR-183, miR-195, miR-199a-5p, microRNAs in the miR-200 family, miR-203, miR-203b-3p, miR-205, miR-206, miR-217, miR-298, miR-375, miR-490-3p, miR-539, miR-542-3p, miR-613, miR-638, miR-940, microRNAs in the let-7 family, anti-miR-9, anti-miR-10b, anti-miR-20a-5p, anti-miR-21, anti-miR-25-3p, anti-miR-26a, anti-miR-27a, anti-miR-29b, anti-miR-30b/30e/30d, anti-miR-31, anti-miR-34a, anti-miR-103, anti-miR-107, anti-miR-122, anti-miR-125b, anti-miR-126, anti-miR-139, anti-miR-143, anti-miR-146a, anti-miR-146b-5p, anti-miR-153, anti-miR-155, anti-miR-181a/b, anti-miR-182, anti-miR-199a-3p, anti-miR-199a-5p, anti-miR-200s, anti-miR-210, anti-miR-214, anti-miR-221, anti-miR-222, anti-miR-223, anti-miR-335, anti-miR-342, anti-miR-373, anti-miR-455-3p, anti-miR-429, anti-miR-493, anti-miR-494, anti-miR-520c, anti-miR-520h, and anti-miR-1908. When an anti-cancer agent is a nucleic acid (e.g., a miRNA or an anti-miRNA), the nucleic acid also can include one or more molecules and/or moieties that can prevent degradation of the nucleic acid (e.g., 2′-O-methyl RNA bases and/or ZEN-end groups). For example, a nucleic acid that is an anti-cancer agent can include one or more locked nucleic acids (LNAs). In some cases, an anti-cancer agent can be as described elsewhere (see, e.g., Piwecka et al., Mol. Oncol., 9:1324-1340 (2015)).

Any appropriate method can be used to include one or more anti-cancer agents into a nanostructure (e.g., a nanotube). In some cases, one or more anti-cancer agents can be intercalated in a nanostructure provided herein (e.g., a nanotube provided herein). For example, one or more hydrophilic anti-cancer agents can be intercalated in a nanostructure provided herein (e.g., a nanotube provided herein). In some cases, one or more anti-cancer agents can be encapsulated within a nanostructure provided herein (e.g., a nanotube provided herein). For example, one or more hydrophobic anti-cancer agents can be encapsulated within a nanostructure provided herein (e.g., a nanotube provided herein). In some cases, an anti-cancer agent can be incorporated into a pre-assembled nanostructure (e.g., nanotube). In some cases, an anti-cancer agent can be hybridized to a pre-assembled nanostructure (e.g., nanotube). In some cases, an anti-cancer agent can be hybridized to one or more NA-amphiphiles that can assemble (e.g., can self-assemble) to form a nanostructure provided herein (e.g., a nanotube provided herein) prior to the NA-amphiphiles assembling (e.g., self-assembling) into the nanostructure.

In some cases, a nanostructure (e.g., a nanotube) provided herein can include, in addition to or as an alternative to one or more anti-cancer agents, one or more therapeutic agents. For example, one or more therapeutic agents can be intercalated in a nanostructure provided herein (e.g., a nanotube provided herein). In some cases, one or more hydrophilic therapeutic agents can be intercalated in a nanostructure provided herein (e.g., a nanotube provided herein). For example, one or more therapeutic agents can be encapsulated within a nanostructure provided herein (e.g., a nanotube provided herein). In some cases, one or more hydrophobic therapeutic agents (e.g., one or more hydrophobic senotherapeutic agents) can be encapsulated within a nanostructure provided herein (e.g., a nanotube provided herein). In some cases, a therapeutic agent can be incorporated into a pre-assembled nanostructure (e.g., nanotube). In some cases, a therapeutic agent can be hybridized to a pre-assembled nanostructure (e.g., nanotube). In some cases, a therapeutic agent can be hybridized to one or more NA-amphiphiles that can assemble (e.g., can self-assemble) to form a nanostructure provided herein (e.g., a nanotube provided herein) prior to the NA-amphiphiles assembling (e.g., self-assembling) into the nanostructure.

In some cases, a nanostructure provided herein (e.g., a nanotube including one or more anti-cancer agents) can include one or more stabilizing molecules. For example, a stabilizing molecule can be conjugated to one or more NA-amphiphiles that can assemble (e.g., can self-assemble) to form a nanostructure provided herein (e.g., a nanotube provided herein) prior to the NA-amphiphiles assembling (e.g., self-assembling) into the nanostructure. For example, a stabilizing molecule can be conjugated to an assembled nanostructure (e.g., nanotube). Examples of stabilizing molecules that can be conjugated to one or more NA-amphiphiles that can assemble (e.g., can self-assemble) to form a nanostructure provided herein (e.g., a nanotube provided herein) include, without limitation, polyethylene glycol (PEG) and polyethylene oxide (PEO).

A nanostructure provided herein (e.g., a nanotube including one or more anti-cancer agents) can be any appropriate size. In cases where a nanostructure provided herein is a nanotube, the nanotube including one or more anti-cancer agents can have a length of from about 20 nm to about 2000 nm (e.g., from about 20 nm to about 1500 nm, from about 20 nm to about 1000 nm, from about 20 nm to about 900 nm, from about 20 nm to about 800 nm, from about 20 nm to about 700 nm, from about 20 nm to about 600 nm, from about 20 nm to about 500 nm, from about 20 nm to about 400 nm, from about 20 nm to about 300 nm, from about 20 nm to about 200 nm, from about 20 nm to about 100 nm, from about 20 nm to about 75 nm, from about 20 nm to about 50 nm, from about 50 nm to about 2000 nm, from about 100 nm to about 2000 nm, from about 200 nm to about 2000 nm, from about 300 nm to about 2000 nm, from about 400 nm to about 2000 nm, from about 500 nm to about 2000 nm, from about 600 nm to about 2000 nm, from about 700 nm to about 2000 nm, from about 800 nm to about 2000 nm, from about 900 nm to about 2000 nm, from about 1000 nm to about 2000 nm, from about 1500 nm to about 2000 nm, from about 50 nm to about 1500 nm, from about 75 nm to about 1000 nm, from about 100 nm to about 500 nm, from about 200 nm to about 300 nm, from about 100 nm to about 300 nm, from about 300 nm to about 500 nm, from about 500 nm to about 800 nm, or from about 800 nm to about 1000 nm). For example, a nanotube including one or more anti-cancer agents can have a length of from about 50 nm to about 650 nm (e.g., 238+122 nm). For example, a nanotube including one or more anti-cancer agents can have a length of from about 195 nm to about 450 nm (e.g., 319+125 nm).

In cases where a nanostructure provided herein is a nanotube, the nanotube can have a diameter (e.g., an outer diameter) of from about 10 nm to about 200 nm (e.g., from about 10 nm to about 175 nm, from about 10 nm to about 150 nm, from about 10 nm to about 125 nm, from about 10 nm to about 100 nm, from about 10 nm to about 80 nm, from about 10 nm to about 60 nm, from about 10 nm to about 50 nm, from about 10 nm to about 40 nm, from about 25 nm to about 200 nm, from about 50 nm to about 200 nm, from about 75 nm to about 200 nm, from about 100 nm to about 200 nm, from about 125 nm to about 200 nm, from about 150 nm to about 200 nm, from about 20 nm to about 180 nm, from about 30 nm to about 160 nm, from about 50 nm to about 150 nm, from about 75 nm to about 125 nm, from about 25 nm to about 50 nm, from about 50 nm to about 100 nm, from about 100 nm to about 125 nm, from about 125 nm to about 150 nm, or from about 150 nm to about 175 nm). For example, a nanotube can have a diameter (e.g., an outer diameter) of from about 20 nm to about 50 nm (e.g., 35+4 nm). For example, a nanotube can have a diameter (e.g., an outer diameter) of from about 30 nm to about 50 nm. In cases where a nanostructure provided herein is a nanotube, the nanotube including one or more anti-cancer agents can have a wall thickness of from about 2 nm to about 20 nm (e.g., from about 2 nm to about 18 nm, from about 2 nm to about 15 nm, from about 2 nm to about 12 nm, from about 2 nm to about 10 nm, from about 2 nm to about 7 nm, from about 2 nm to about 5 nm, from about 5 nm to about 20 nm, from about 8 nm to about 20 nm, from about 10 nm to about 20 nm, from about 12 nm to about 20 nm, from about 15 nm to about 20 nm, from about 17 nm to about 20 nm, from about 5 nm to about 17 nm, from about 8 nm to about 15 nm, from about 10 nm to about 12 nm, from about 5 nm to about 10 nm, from about 10 nm to about 15 nm, or from about 15 nm to about 18 nm). For example, a nanotube including one or more anti-cancer agents can have a wall thickness of from about 4 nm to about 12 nm (e.g., 8+2 nm)

In some cases, a nanostructure provided herein (e.g., a nanotube including one or more anti-cancer agents) can be conjugated to a detectable label. For example, a detectable label can be conjugated to an assembled nanostructure provided herein. For example, a detectable label can be conjugated to one or more NA-amphiphiles that can assemble (e.g., can self-assemble) to form a nanostructure provided herein prior to the NA-amphiphiles assembling (e.g., self-assembling) into a nanostructure. In some cases, a detectable label can be radioactive. In some cases, a detectable label can be fluorescent. In some cases, a detectable label can be luminescent. In some cases, a detectable label can be a dye. A non-limiting example of a detectable label that can be conjugated to a nanostructure provided herein is ⁶⁴Cu-DOTA. A detectable label can be conjugated to a nanostructure provided herein at any appropriate location. In cases, a detectable label can be conjugated to the 5′ end of an NA-amphiphile that can assemble to form a nanostructure provided herein. In cases, a detectable label can be conjugated to the 3′ end of an NA-amphiphile that can assemble to form a nanostructure provided herein. In cases, a detectable label can be conjugated to an end of an NA-amphiphile that can assemble to form a nanostructure provided herein that is exposed at the interface. In cases where a detectable label is conjugated to a NA-amphiphile that can assemble to form a nanostructure provided herein, a detectable label can be conjugated to the NA headgroup of the NA-amphiphile.

In some cases, a nanostructure provided herein (e.g., a nanotube including one or more anti-cancer agents) can lack any targeting molecule. For example, a nanostructure provided herein can lack a targeting molecule typically used to target a cancer treatment to a cancer cell within a mammal (e.g., a human).

In some cases, a nanostructure provided herein (e.g., a nanotube including one or more anti-cancer agents) can be stable. For example, a nanostructure provided herein can be stable in the presence of (e.g., is not degraded by) one or more enzymes typically present in the body of mammal that the nanostructure provided herein can be administered to (e.g., a mammal such as a human having cancer). In some cases, a nanostructure provided herein is not degraded by one or more endonuclease polypeptides (e.g., DNase polypeptides such as DNase I polypeptides and RNase polypeptides such as RNase 1 polypeptides). In some cases, a nanostructure provided herein is not degraded by one or more exonuclease polypeptides (e.g., exonuclease III polypeptides).

In some cases, a nanostructure provided herein (e.g., a nanotube including one or more anti-cancer agents) can be coated, at least in part, with one or more layers. For example, a nanostructure provided herein can be coated, at least in part, with a layer including one or more polymers. In some cases, a polymer that can be included in a layer coating at least part of a nanostructure provided herein can be a polysaccharide-based polymer. Examples of polymers that can be included in a layer coating at least part of a nanostructure provided herein include, without limitation, polyethylenimine (PEI), poly(allylamine), polyamine-based polymers, polylysine, polyarginine, polyglutamic acid, polyamino esters, polymethacrylates, cyclodextrin-based polymers, polysaccharides (e.g., fucoidan, chitosan, hyaluronic acid, dextran, dextran sulfate, p-cyclodextrin, cyclodextrins, alginic acid, alginate, cellulose sulfate, cellulose, heparin, protamine sulfate, and carboxymethylcellulose), poly(styrene sulfonate), poly(dimethyldiallylammonium chloride), poly(N-isopropyl acrylamide), poly(acrylic acid), poly(methacrylic acid), poly(vinyl sulfate), poly(ethylene oxide), and poly(ethylene glycol). In some cases, a layer including one or more polymers and coating at least part of a nanostructure provided herein can promote escape of the nanostructure from endosomes and/or lysosomes after cell internalization.

In some cases, a nanostructure provided herein can be coated, at least in part, with a layer including one or more targeting molecules. Examples of molecules that can be targeted by a targeting molecule that can be included in a layer coating at least part of a nanostructure provided herein include, without limitation, scavenger receptors, toll-like receptors, C-type lectins, selectins, integrins, vascular endothelial growth factors, vascular endothelial growth factor receptors, chemokines, elastin peptide receptors, extracellular matrix proteins, and transforming growth factor-β (TGF-β) polypeptides. In some cases, a nanostructure provided herein can be coated, at least in part, with a layer including one or more random (e.g., a non-targeting) sequences. Examples of random (e.g., a non-targeting) sequences that can be included in a layer coating at least part of a nanostructure provided herein include, without limitation, CTCTTGGGGG (SEQ ID NO:1) and GGGGGTTCTC (SEQ ID NO:2). In some cases, a nanostructure provided herein can be coated, at least in part, with a layer including one or more polysaccharides. Examples of polysaccharides that can be included in a layer coating at least part of a nanostructure provided herein include, without limitation, fucoidan, chitosan, hyaluronic acid, dextran, dextran sulfate, β-cyclodextrin, cyclodextrins, alginic acid, alginate, cellulose sulfate, cellulose, heparin, protamine sulfate, and carboxymethylcellulose.

In some cases, a nanostructure provided herein can be coated, at least in part, with a layer including one or more miRNAs. Examples of miRNAs that can be targeted by a targeting molecule that can be included in a layer coating at least part of a nanostructure provided herein include, without limitation, miR-603, miR-34a, miR-21, miR-218, miR-219, miR-183m, miR-451, miR-133, miR-134, miR-302c, miR-324, miR-379, miR-491, miR-340, miR-7, miR-128, miR-368-3p, miR-10b, miR-15a, miR-16, miR-17-5p, miR-26a, miR-29, miR-29b, miR-31, miR-33a, miR-34, miR-93, miR-101, miR-101-3p, miR-122, miR-122a, miR-125b, miR-130a, miR-133-b, miR-136, miR-143, miR-145, miR-146a-5p, miR-148a, miR-181d, miR-182, miR-183, miR-195, miR-199a-5p, microRNAs in the miR-200 family, miR-203, miR-203b-3p, miR-205, miR-206, miR-217, miR-298, miR-375, miR-490-3p, miR-539, miR-542-3p, miR-613, miR-638, miR-940, and microRNAs in the let-7 family.

In some cases, a nanostructure provided herein can be coated, at least in part, with a layer including one or more anti-miRNAs. Examples of anti-miRNAs that can be targeted by a targeting molecule that can be included in a layer coating at least part of a nanostructure provided herein include, without limitation, anti-miR-9, anti-miR-10b, anti-miR-20a-5p, anti-miR-21, anti-miR-25-3p, anti-miR-26a, anti-miR-27a, anti-miR-29b, anti-miR-30b/30e/30d, anti-miR-31, anti-miR-34a, anti-miR-103, anti-miR-107, anti-miR-122, anti-miR-125b, anti-miR-126, anti-miR-139, anti-miR-143, anti-miR-146a, anti-miR-146b-5p, anti-miR-153, anti-miR-155, anti-miR-181a/b, anti-miR-182, anti-miR-199a-3p, anti-miR-199a-5p, anti-miR-200s, anti-miR-210, anti-miR-214, anti-miR-221, anti-miR-222, anti-miR-223, anti-miR-335, anti-miR-342, anti-miR-373, anti-miR-455-3p, anti-miR-429, anti-miR-493, anti-miR-494, anti-miR-520c, anti-miR-520h, and anti-miR-1908.

In some cases, a nanostructure provided herein (e.g., a nanotube including one or more anti-cancer agents) can be as described elsewhere (see, e.g., U.S. Pat. No. 10,415,040; Pearce et al., Chem. Commun., 50: 210-212 (2014); Pearce et al., Soft Matter., 11:109-117 (2015); and Kuang et al., Nanoscale, 11:19850-19861 (2019)).

Any appropriate method can be used to make a nanostructure provided herein (e.g., a nanotube including one or more anti-cancer agents). In some cases, a nanostructure provided herein can self-assemble. In some cases, DNA origami and/or DNA tile assembly can be used to make a nanostructure provided herein. In some cases, a nanostructure provided herein can be as described elsewhere (see, e.g., Rothemund, Nature, 440:297-302 (2006); and Yan, Science, 301:1882-1884 (2003)). In some cases, methods for making a nanostructure provided herein (e.g., a nanotube including one or more anti-cancer agents) can include isolating the nanostructure. For example, when a nanostructure is a nanotube, methods for making the nanotube can include isolating the nanotubes from other nanostructures (e.g., micelles such as spherical micelles and/or cylindrical micelles) that may also form during the self-assembly process. In some cases, methods for making a nanostructure provided herein (e.g., a nanotube including one or more anti-cancer agents) can include altering (e.g., shortening) the length of the nanostructure. For example, when a nanostructure is a nanotube, methods for making the nanotube can include sonication (e.g., probe sonication) to shorten the length of the nanotube.

In some cases, nanostructures provided herein (e.g., nanotubes including one or more anti-cancer agents) can be made using a layer-by-layer (LBL) synthesis. For example, a LBL synthesis can be used to make a nanostructure provided herein that is coated, at least in part, with a one or more layers.

In some cases, a nanostructure provided herein (e.g., a nanotube including one or more anti-cancer agents) can be produced from micelles (e.g., spherical micelles). For example, a population of one or more NA-amphiphiles described herein can be neutralized, precipitated, and dried. The dried precipitate can then be combined with dialkyl tail molecules (e.g., dialkyl tail molecules attached to a spacer) to form a nanostructure provided herein.

In some cases, methods for making a nanostructure provided herein (e.g., a nanotube including one or more anti-cancer agents) can be as described in any one or more of Examples 1-9.

In some cases, nanostructures provided herein (e.g., nanotubes including one or more anti-cancer agents) can be formulated into a composition (e.g., a pharmaceutically acceptable composition). For example, a composition including nanostructures provided herein can include one or more pharmaceutically acceptable carriers (additives), excipients, and/or diluents. Examples of pharmaceutically acceptable carriers, excipients, and diluents that can be used in a composition described herein include, without limitation, PEGs, phosphate-buffered saline (PBS), polymers (e.g., thermosensitive polymers and biodegradable polymers), water, salts or electrolytes (e.g., saline, protamine sulfate, disodium hydrogen phosphate, potassium hydrogen phosphate, sodium chloride, potassium chloride, calcium chloride, magnesium chloride, manganese chloride, and zinc salts), colloidal silica, magnesium trisilicate, polyacrylates, waxes, wool fat, and lecithin. A pharmaceutical composition can be formulated for administration in solid or liquid form including, without limitation, sterile solutions, suspensions, sustained-release formulations, tablets, capsules, pills, powders, and granules.

In some cases, a composition including nanostructures provided herein (e.g., nanotubes including one or more anti-cancer agents) can be formulated as a delivery system. For example, a composition including nanostructures provided herein can be formulated as a controlled-release delivery system for the one or more anti-cancer agents. Examples of types of controlled-release delivery that a composition including nanoparticles described herein can be formulated for include, without limitation, induced release, burst release, slow release, delayed release, and sustained release.

This document also provides methods and materials for using nanostructures provided herein (e.g., nanotubes including one or more anti-cancer agents). In some cases, a mammal (e.g., a human) having cancer (e.g., a CNS cancer such as GBM or a TNBC) can be treated by administering nanostructures provided herein (e.g., a composition including nanostructures provided herein) to the mammal.

Any type of mammal having or having cancer (e.g., a CNS cancer such as GBM or a TNBC) can be treated using the methods and materials described herein (e.g., by administering nanostructures such as nanotubes including one or more anti-cancer agents). Examples of mammals that can be treated as described herein include, without limitation, humans, non-human primates such as monkeys, dogs, cats, horses, cows, pigs, sheep, rabbits, mice, and rats. In some cases, a human having cancer (e.g., a CNS cancer such as GBM or a TNBC) can be treated by administering nanostructures provided herein (e.g., nanotubes including one or more anti-cancer agents).

In some cases, the methods described herein can include identifying a mammal (e.g., a human) as having cancer (e.g., a CNS cancer such as GBM or a TNBC). Any appropriate method can be used to identify a mammal as having cancer (e.g., a glioma such as GBM or a TNBC). For example, medical history (e.g., a history of having had a prior CNS cancer), neurological examinations (e.g., to check vision, hearing, balance, coordination, strength, and/or reflexes), imaging techniques such as magnetic resonance imaging (MRI), magnetic resonance spectroscopy, computed tomography (CT) scanning, and positron emission tomography (PET) scanning (e.g., to determine the location and size of a brain tumor), and/or biopsy techniques can be used to identify mammals (e.g., humans) having, or at risk of developing, a cancer (e.g., a CNS cancer such as GBM or a TNBC).

A mammal (e.g., a human) having any type of cancer can be treated as described herein (e.g., by administering nanostructures such as nanotubes including one or more anti-cancer agents). In some cases, a cancer can be a blood cancer (e.g., lymphomas and leukemias). In some cases, a cancer can include one or more solid tumors. In some cases, a cancer can be a primary cancer. In some cases, a cancer can be a metastatic cancer. In some cases, a cancer can be a recurrent cancer. In some cases, a cancer can be a chemotherapy-resistant cancer. In some cases, a cancer can be a CNS cancer. Examples of cancers that can be treated as described herein include, without limitation, gliomas (e.g., brain stem gliomas and GBMs), astrocytomas, oligodendrogliomas, oligoastrocytomas, ependymomas, medulloblastomas, meningiomas, diffuse intrinsic pontine glioma (DIPG), breast cancers (e.g., TNBCs), colon cancers, liver cancer, pancreatic cancer, prostate cancer, lung cancer, ovarian cancer, kidney cancer, spleen cancer, and gastric cancer.

In some cases, nanostructures provided herein (e.g., nanotubes including one or more anti-cancer agents) can be administered to a mammal in need thereof (e.g., a mammal having cancer such as a human having a CNS cancer such as GBM or a TNBC) to reduce or eliminate the number of cancer cells present within a mammal. In some cases, nanostructures provided herein can be administered to a mammal having cancer (e.g., a CNS cancer such as GBM or a TNBC) to reduce or eliminate the number of senescent cancer cells present within the mammal. In some cases, nanostructures provided herein can be administered to a mammal having cancer (e.g., a CNS cancer such as GBM or a TNBC) to reduce or eliminate the number of proliferating cancer cells present within the mammal. For example, the materials and methods described herein can be used to reduce the number of cancer cells present within a mammal having cancer by, for example, 10, 20, 30, 40, 50, 60, 70, 80, 90, 95, or more percent. For example, the materials and methods described herein can be used to reduce the size (e.g., volume) of one or more tumors present within a mammal having cancer (e.g., a CNS cancer such as GBM or a TNBC) by, for example, 10, 20, 30, 40, 50, 60, 70, 80, 90, 95, or more percent.

In some cases, nanostructures provided herein (e.g., nanotubes including one or more anti-cancer agents) can be administered to a mammal in need thereof (e.g., a mammal having cancer such as a human having a CNS cancer such as GBM or a TNBC) to improve survival of the mammal. For example, disease-free survival (e.g., recurrence-free survival) can be improved using the materials and methods described herein. For example, progression-free survival can be improved using the materials and methods described herein.

In some cases, the materials and methods described herein can be used to improve the survival of a mammal having cancer (e.g., a CNS cancer such as GBM or a TNBC) by, for example, 10, 20, 30, 40, 50, 60, 70, 80, 90, 95, or more percent.

In some cases, nanostructures provided herein (e.g., nanotubes including one or more anti-cancer agents) can be administered to a mammal in need thereof (e.g., a mammal having cancer such as a human having a CNS cancer such as GBM or a TNBC) to repolarize one or more TAMs (e.g., one or more TAMs having a M2-phenotype) within the mammal.

For example, nanostructures provided herein can be administered to a mammal (e.g., a human) having cancer (e.g., a CNS cancer such as GBM or a TNBC) to repolarize TAMs present within the mammal to an M1-phenotype. In some cases, the materials and methods described herein can be used to repolarize, for example, 10, 20, 30, 40, 50, 60, 70, 80, 90, 95, or more percent of the TAMs present within a mammal having cancer (e.g., a CNS cancer such as GBM or a TNBC) to an M1-phenotype.

Any appropriate method can be used to administer nanostructures provided herein (e.g., nanotubes including one or more anti-cancer agents) to a mammal (e.g., a human) having cancer (e.g., a CNS cancer such as GBM or a TNBC). For example, a composition including nanostructures provided herein can be designed for oral or parenteral (including, without limitation, a subcutaneous, intramuscular, intracranial, intravenous, intradermal, intra-cerebral, intrathecal, or intraperitoneal injection) administration to a mammal having cancer. Compositions suitable for parenteral administration include, without limitation, aqueous and non-aqueous sterile injection solutions that can contain anti-oxidants, buffers, bacteriostats, and solutes that render the formulation isotonic with the blood of the intended recipient. In some cases, a composition including nanostructure provided herein can be designed for use with a delivery system (e.g., a convection-enhanced delivery (CED) system such as a pump).

In some cases, nanostructures provided herein (e.g., nanotubes including one or more anti-cancer agents), when administered to a mammal (e.g., a human), can cross the blood brain barrier. For example, a composition including nanostructures provided herein, when administered to a mammal (e.g., a human), can cross the blood brain barrier and enter the brain of that mammal thereby delivering the nanostructures provided herein to the brain of that mammal.

In some cases, nanostructures provided herein (e.g., nanotubes including one or more anti-cancer agents) can accumulate in a tissue containing cancers cells within a mammal having cancer (e.g., a CNS cancer such as GBM or a TNBC).

Nanostructures provided herein (e.g., nanotubes including one or more anti-cancer agents) can be administered to a mammal (e.g., a human) having cancer (e.g., a CNS cancer such as GBM or a TNBC) in any appropriate amount (e.g., any appropriate dose). For example, an effective amount of a composition containing nanostructures provided herein can be any amount that can treat a mammal having cancer as described herein without producing significant toxicity (e.g., systemic toxicity) to the mammal. For example, when nanotubes including doxorubicin are administered to a mammal (e.g., a human) having cancer (e.g., a CNS cancer such as GBM or a TNBC), an effective amount of nanotubes can include from about 5 milligrams doxorubicin per body surface area of the mammal (mg/m²) to about 60 mg/m² (e.g., from about 5 mg/m² to about 55 mg/m², from about 5 mg/m² to about 50 mg/m², from about 5 mg/m² to about 45 mg/m², from about 5 mg/m² to about 40 mg/m², from about 5 mg/m² to about 35 mg/m², from about 5 mg/m² to about 30 mg/m², from about 5 mg/m² to about 25 mg/m², from about 5 mg/m² to about 20 mg/m², from about 5 mg/m² to about 15 mg/m², from about 5 mg/m² to about 10 mg/m², from about 10 mg/m² to about 60 mg/m², from about 15 mg/m² to about 60 mg/m², from about 20 mg/m² to about 60 mg/m², from about 25 mg/m² to about 60 mg/m², from about 30 mg/m² to about 60 mg/m², from about 35 mg/m² to about 60 mg/m², from about 40 mg/m² to about 60 mg/m², from about 45 mg/m² to about 60 mg/m², from about 50 mg/m² to about 60 mg/m², from about 55 mg/m² to about 60 mg/m², from about 10 mg/m² to about 50 mg/m², from about 20 mg/m² to about 40 mg/m², from about 10 mg/m² to about 30 mg/m², or from about 30 mg/m² to about 50 mg/m²) of doxorubicin (e.g., per month). The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and/or severity of the cancer may require an increase or decrease in the actual effective amount administered.

Nanostructures provided herein (e.g., nanotubes including one or more anti-cancer agents) can be administered to a mammal (e.g., a human) having cancer (e.g., a CNS cancer such as GBM or a TNBC) in any appropriate frequency. The frequency of administration can be any frequency that can treat a mammal having cancer without producing significant toxicity (e.g., systemic toxicity) to the mammal. For example, the frequency of administration can be from about once a week to about once a month, from about once a week to about once every two weeks, or from about once a month to about once every two months. The frequency of administration can remain constant or can be variable during the duration of treatment. As with the effective amount, various factors can influence the actual frequency of administration used for a particular application. For example, the effective amount, duration of treatment, use of multiple treatment agents, and/or route of administration may require an increase or decrease in administration frequency.

Nanostructures provided herein (e.g., nanotubes including one or more anti-cancer agents) can be administered to a mammal (e.g., a human) having cancer (e.g., a CNS cancer such as GBM or a TNBC) for any appropriate duration. An effective duration for administering or using a composition containing nanostructures provided herein can be any duration that can treat a mammal having cancer without producing significant toxicity (e.g., systemic toxicity) to the mammal. For example, the effective duration can vary from several weeks to several months, from several months to a year, or for a year or more. Multiple factors can influence the actual effective duration used for a particular treatment. For example, an effective duration can vary with the frequency of administration, effective amount, use of multiple treatment agents, and/or route of administration.

In some cases, nanostructures provided herein (e.g., nanotubes including one or more anti-cancer agents) can be administered to a mammal in need thereof (e.g., a mammal having cancer such as a human having a CNS cancer such as GBM or a TNBC) as the sole active ingredient used to the treat the mammal. For example, a composition containing nanostructures provided herein can include the nanostructures as the sole active ingredient in the composition that is effective to treat a mammal having cancer (e.g., a CNS cancer such as GBM or a TNBC).

In some cases, methods for treating a mammal (e.g., a human) having cancer (e.g., a CNS cancer such as GBM or a TNBC) as described herein (e.g., by administering nanostructures such as nanotubes including one or more anti-cancer agents) can include administering to the mammal nanostructures provided herein (e.g., nanotubes including one or more anti-cancer agents) together with one or more (e.g., one, two, three, four, five or more) senotherapeutic agents. In some cases, a senotherapeutic agent can be a senolytic agent (i.e., an agent having the ability to induce cell death in senescent cells). In some cases, a senotherapeutic agent can be a senomorphic agent (i.e., an agent having the ability to suppress senescent phenotypes without cell killing). In some cases, a senotherapeutic agent can be an inhibitor of a BCL-2 polypeptide. Examples of senotherapeutic agents that can be administered together with nanostructures provided herein include, without limitation, ABT-263, ABT-199, A1155463, A1331852, dasatinib, quercetin, methadone, and any combinations thereof. In cases where nanostructures provided herein are used in combination with one or more senotherapeutic agents, the one or more senotherapeutic agents can be administered at the same time (e.g., as nanostructures including both the one or more anti-cancer agents and the one or more senotherapeutic agents or in a single composition containing both nanostructures provided herein and the one or more senotherapeutic agents) or independently. For example, nanostructures provided herein can be administered first, and the one or more senotherapeutic agents administered second, or vice versa.

In some cases, methods for treating a mammal (e.g., a human) having cancer (e.g., a CNS cancer such as GBM or a TNBC) as described herein (e.g., by administering nanostructures such as nanotubes including one or more anti-cancer agents) can include administering to the mammal nanostructures provided herein (e.g., nanotubes including one or more anti-cancer agents) together with one or more (e.g., one, two, three, four, five or more) additional anti-cancer agents (e.g., chemotherapeutic agents) used to treat cancer. Examples of anti-cancer agents that can be administered together with nanostructures provided herein include, without limitation, doxorubicin, epirubicin, tamoxifen, paclitaxel, docetaxel gemcitabine, 5FU, carboplatin, cyclophosphamide temozolomide, cisplatin, IPI-549, camptothecin, curcumin, dexamethasone, furosemide, oxaliplatin, and KPT-9274. In cases where nanostructures provided herein are used in combination with additional agents used to treat cancer, the one or more additional agents can be administered at the same time (e.g., in a single composition containing both nanostructures provided herein and the one or more additional agents) or independently. For example, nanostructures provided herein can be administered first, and the one or more additional agents administered second, or vice versa.

In some cases, methods for treating a mammal (e.g., a human) having cancer (e.g., a CNS cancer such as GBM or a TNBC) as described herein (e.g., by administering nanostructures such as nanotubes including one or more anti-cancer agents) can include administering to the mammal nanostructures provided herein (e.g., nanotubes including one or more anti-cancer agents) together with one or more (e.g., one, two, three, four, five or more) additional therapies used to treat cancer. Examples of therapies that can be used to treat cancer include, without limitation, surgery, radiation therapy, and immunotherapies (e.g., CAR-T cell therapies). In cases where nanostructures provided herein are used in combination with one or more additional therapies used to treat cancer, the one or more additional therapies can be performed at the same time or independently of the administration of the nanostructures provided herein. For example, the nanostructures provided herein can be administered before, during, or after the one or more additional therapies are performed.

The invention will be further described in the following examples, which do not limit the scope of the invention described in the claims.

### EXAMPLES

### Example 1: Self-assembled ssDNA Nanotubes for Selective Targeting of Intracranial Glioblastoma and Delivery of Doxorubicin

This Example demonstrates that nanotubes formed from the self-assembly of short single-stranded DNA (ssDNA)-amphiphiles can be used to deliver doxorubicin (DOX) across the blood brain barrier (BBB; e.g., the blood brain tumor barrier (BBTB)) to treat GBM. For example, treatment of mice with orthotopic GBM with DOX intercalated in the nanotubes resulted in enhanced survival with no systemic toxicity.

### Results and Discussion

Synthesis and Characterization of ssDNA-Amphiphile Nanotubes

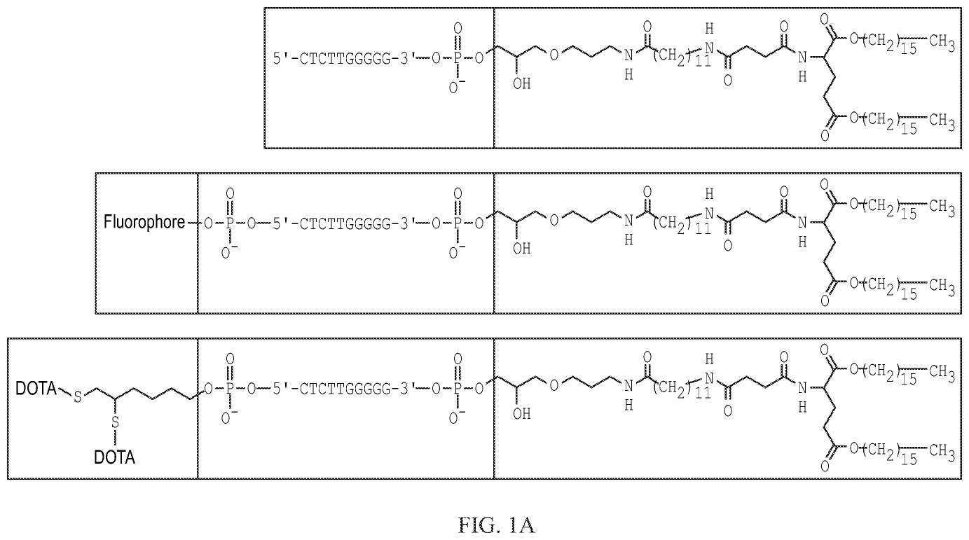

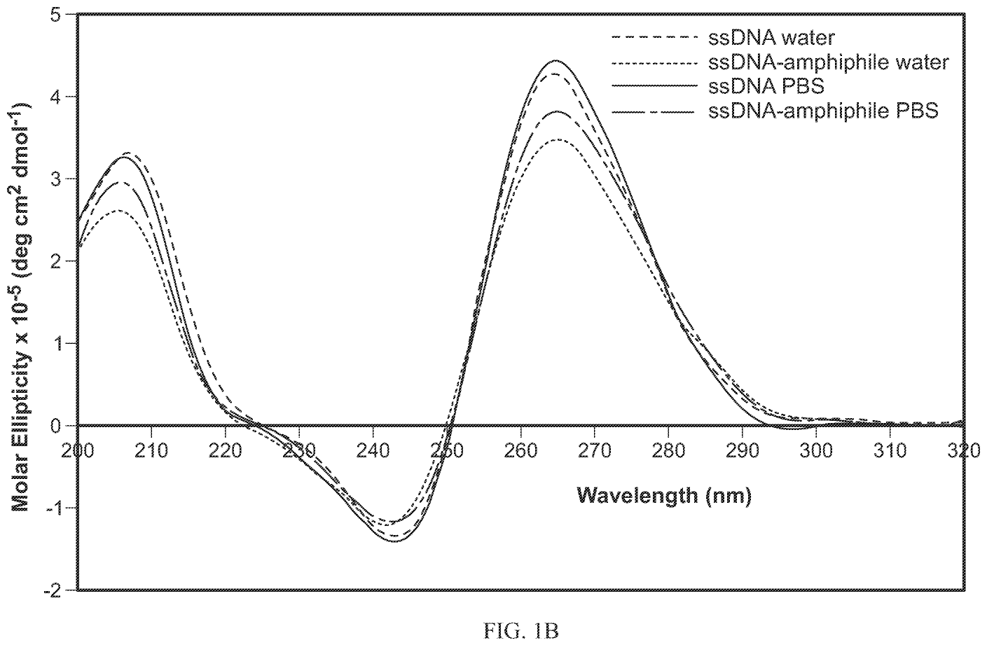

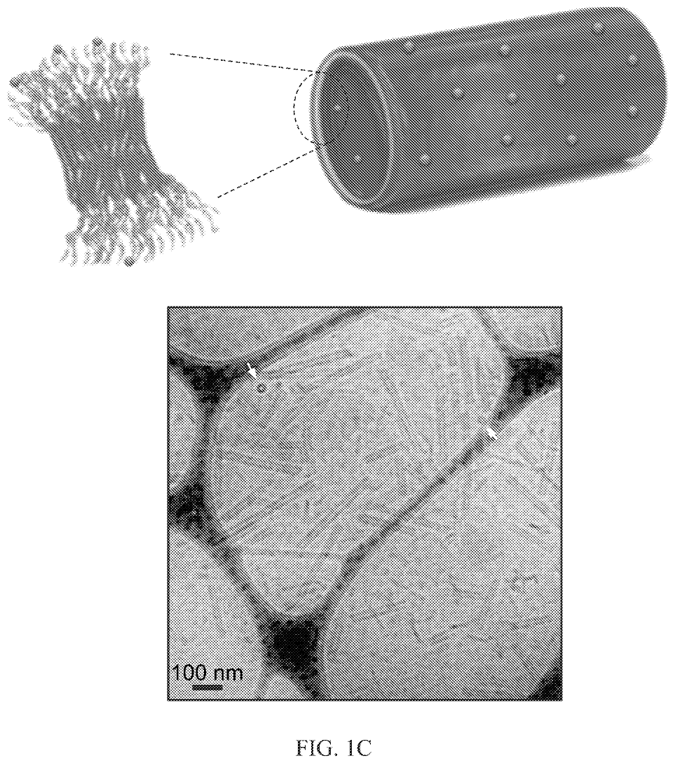

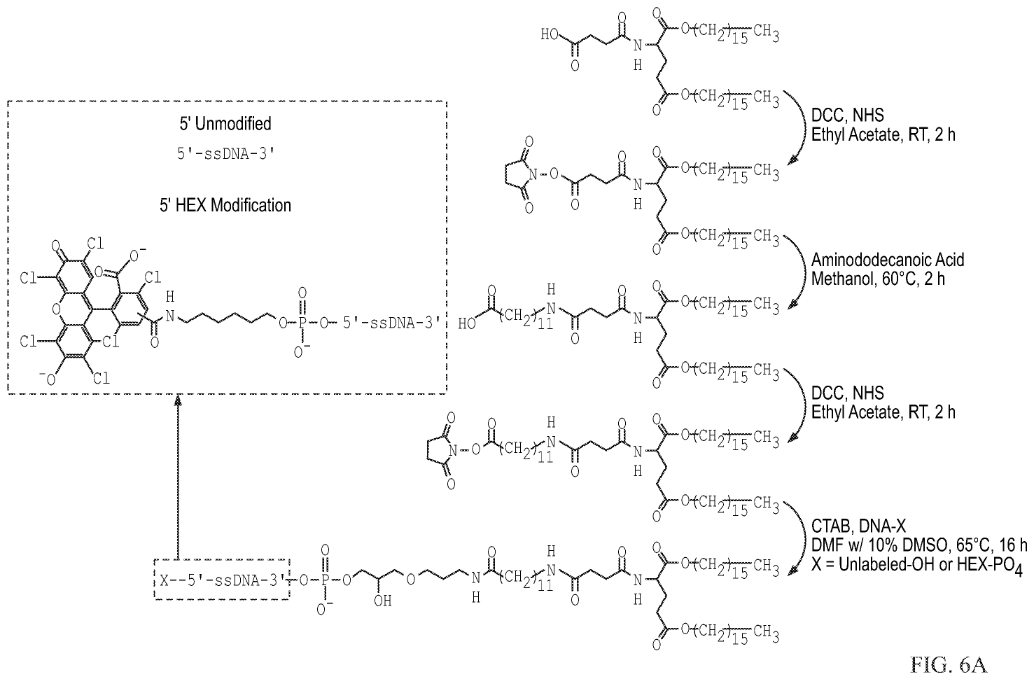

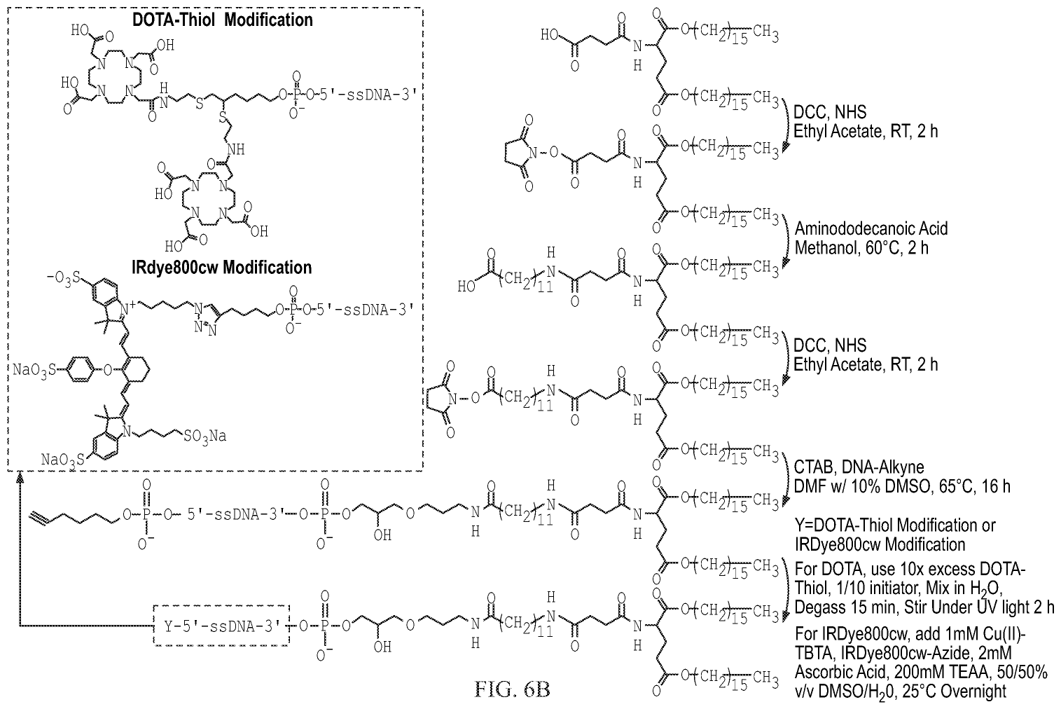

Synthesis steps for all ssDNA-amphiphiles are shown in FIG. 6. Successful synthesis was verified using liquid chromatography-mass spectrometry (Table 1). FIG. 1A shows the chemical structure of the 10 nucleotide (nt) ssDNA-amphiphile, with and without a fluorophore and chelating agent. The secondary structure of the free ssDNA and nanotubes assembled from the ssDNA-amphiphiles was investigated by circular dichroism (CD). The CD spectra of the free 10nt ssDNA and ssDNA nanotubes in Milli-Q water and phosphate buffered saline (PBS) have maxima at 206 and 265 nm and a minimum at 243 nm (FIG. 1i), characteristic of a parallel G-quadruplex structure. Conjugation of the 10 nt ssDNA to a hydrophobic tail and self-assembly did not alter its secondary structure. The 10 nt ssDNA-amphiphiles form weakly ellipsoidal micelles and hollow nanotubes in water, as demonstrated via cryogenic transmission electron microscopy (cryo-TEM) and small angle X-ray scattering. In this study, the nanotubes were separated from the micelles using size exclusion chromatography. The nanotubes were 238±122 nm long, with a diameter of 35±4 nm and a bilayer wall thickness of 8±2 nm (n=100), as evaluated by cryo-TEM images (FIG. 1C). The white arrows in FIG. 1C point to short nanotubes that are viewed end-on, demonstrating the hollow nature of the ssDNA-amphiphile nanotubes.

TABLE 1
Masses of ssDNA-amphiphiles as determined by LC-MS.

| | Expected mass | Measured mass |
|---|---|---|
| ssDNA-amphiphiles | (Da) | (Da) |
| 10 nt ssDNA-amphiphile | 4161.6 | 4159.3 |
| HEX-10 nt ssDNA-amphiphile | 4905.7 | 4901.2 |
| DOTA-10 nt ssDNA-amphiphile | 5245.8 | 5243.7 |

Preferential GL261 Uptake of ssDNA Nanotubes Via Scavenger Receptors and Micropinocytosis

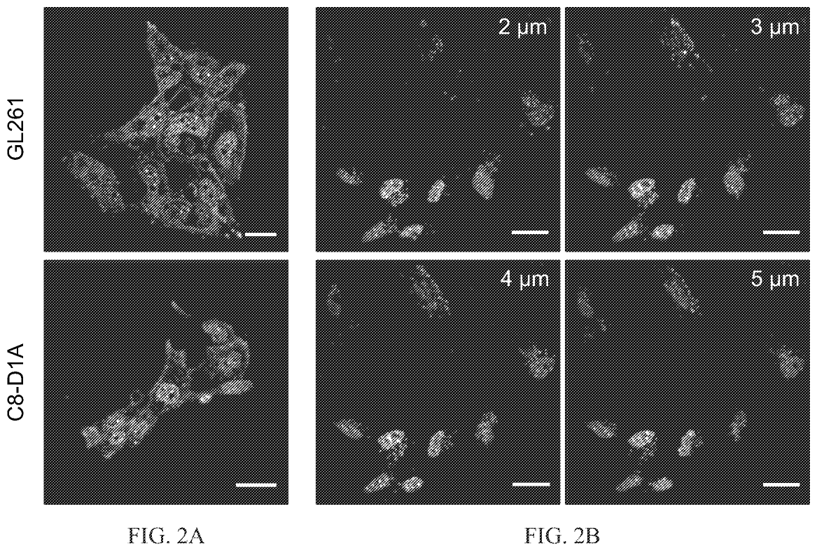

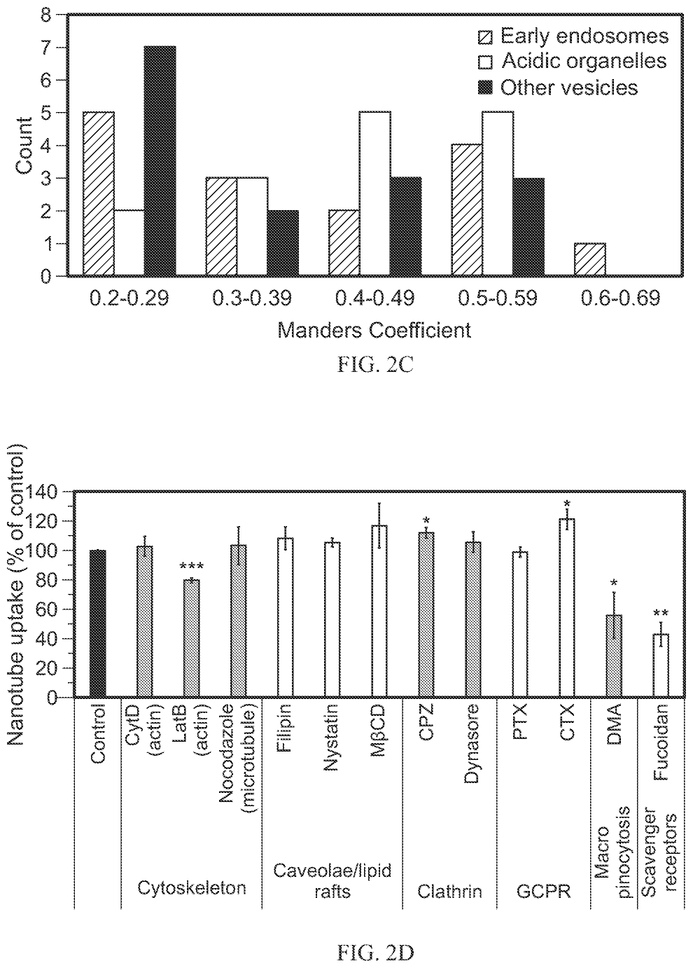

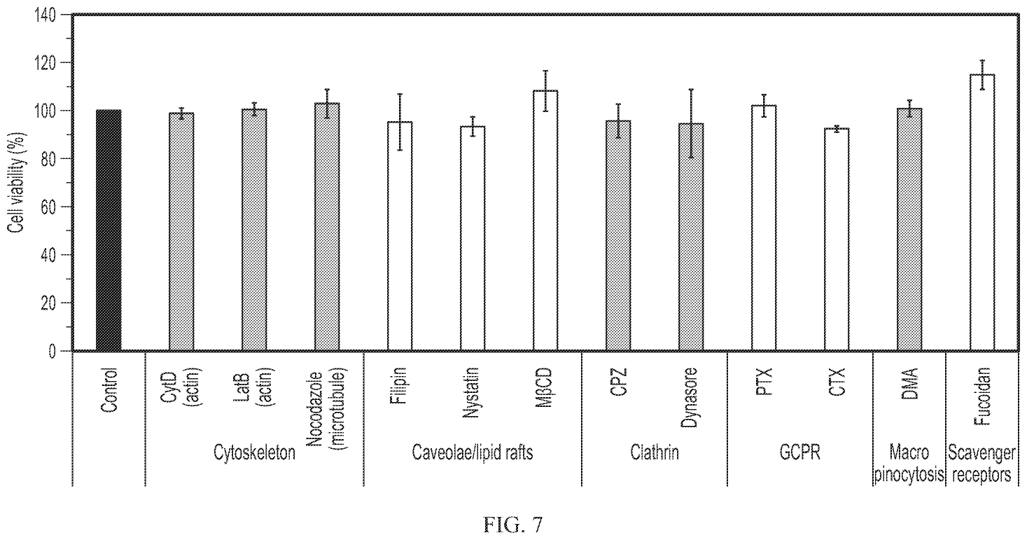

The interaction between the 10 nt ssDNA-amphiphile nanotubes with GL261 mouse GBM cells and C8-D1A healthy mouse astrocytes was studied. HEX-labeled nanotubes were incubated with the cells for 24 hours at 37° C. and confocal microscopy was used to determine qualitatively the extent of cell internalization (FIG. 2a). The nanotubes showed strong cell internalization into the GL261 cells with minimal surface binding and no internalization into the C8-D1A cells. The intracellular fate of the nanotubes was further determined by incubating GL261 cells with the nanotubes for 24 hours and staining for early endosomes and acidic organelles, such as late endosomes and lysosomes. Results showed that after 24 hours the nanotubes were colocalized with early endosomes and acidic organelles as indicated by the magenta and yellow color observed in the images respectively (FIG. 2B). In addition, dots were also located in the cytosol, not associated with early endosomes or acidic organelles, suggesting that the nanotubes may also be located in non-acidic or moderately acidic vesicles. Calculation of the Manders coefficient (FIG. 2C) verified these observations. To reveal the cellular internalization mechanism of the nanotubes, an endocytosis inhibition experiment was performed where the cellular uptake of the ssDNA nanotubes was evaluated in GL261 cells in the presence of different endocytosis inhibitors that did not induce any toxicity to the cells (FIG. 2D and FIG. 7). The inhibitors used can be classified into six major groups based on their effects in cells: cytoskeleton (cytochalasin D (CytD) disrupts actin microfilaments, latrunculin B (LatB) inhibits actin polymerization, and nocodazole disrupts microtubule assembly), caveolae/lipid rafts (filipin, nystatin, and methyl-o-cyclodextrin (MPCD) inhibit caveolae and lipid raft internalization through depletion of cholesterol from the cell membrane by forming inclusion complexes with cholesterol), clathrin (chlorpromazine (CPZ) prevents the assembly and disassembly of clathrin lattices on cell surfaces, and dynasore is an inhibitor of dynamin that participates in clathrin-mediated endocytosis), G-protein coupled receptors (GPCR) (pertussis toxin (PTX) is an inhibitor of Gαi-protein and cholera toxin (CTX) is an inhibitor of Gat/s-proteins), macropinocytosis (5-(N,N-dimethyl)-amiloride hydrochloride (DMA) inhibits Na*/H+exchanger activity), and scavenger receptors (fucoidan is a polysaccharide that binds to various types of scavenger receptors). The cellular update of the nanotubes was inhibited on average by 44% in the presence of DMA and 57% in the presence of fucoidan. In addition, treatment with LatB decreased the internalization of the ssDNA nanotubes by 20%, consistent with the finding that macropinocytosis is involved in the internalization of the nanotubes, since macropinocytosis depends on actin polymerization. In contrast, neither caveolae/lipid rafts, clathrin, or GPCR-associated pathway inhibitors decreased nanotube internalization. These results demonstrate that the ssDNA nanotubes bind to scavenger receptors on GL261 cells followed by cell internalization through macropinocytosis, thus providing a strategy to target the macropinocytosis pathway and provide a novel therapeutic approach to treat GBM.

### Nanotube Stability and In Vivo Tumor Targeting

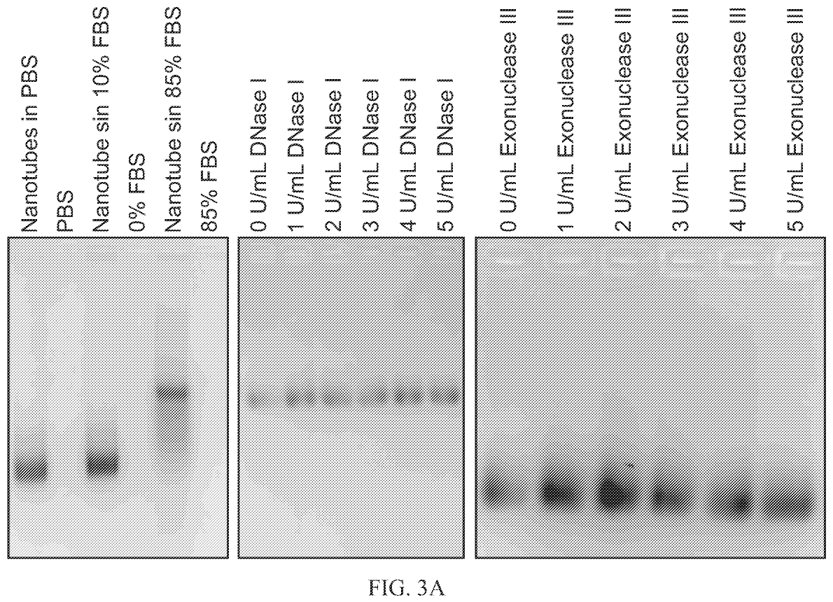

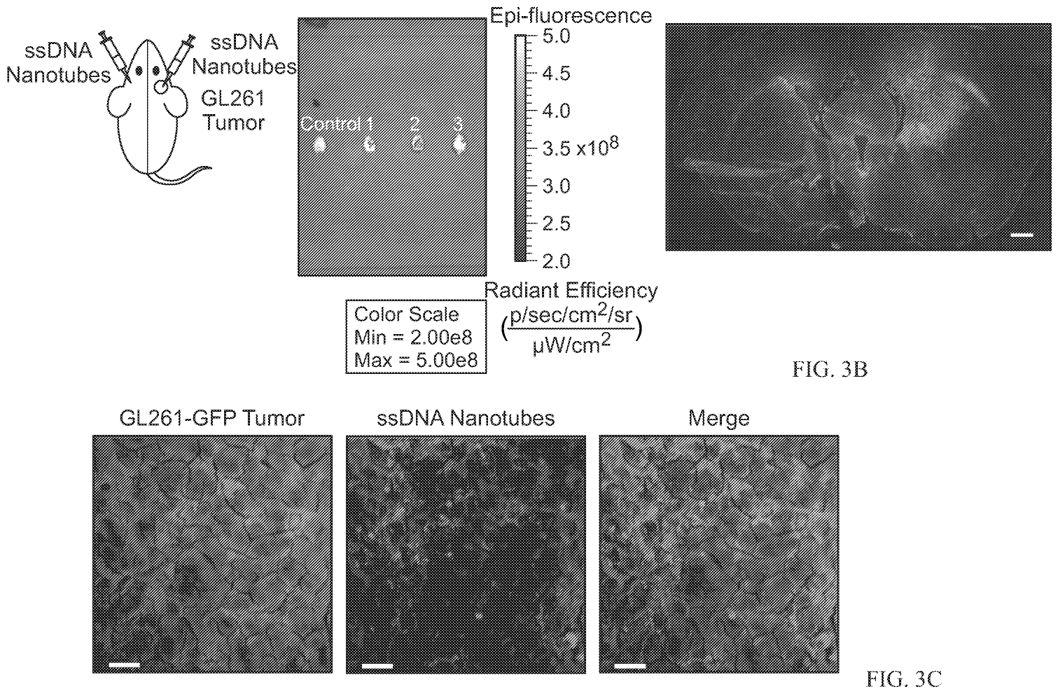

A common limitation of ssDNA-based nanoparticles is their stability when delivered in vivo. The main degradation pathways as reported in the literature are through desorption of ssDNA by serum proteins, and degradation by nucleases, where direct cleavage of ssDNA at an internal site by endonucleases, or removal of nucleotides at the terminus by exonucleases is a possibility. The stability of the nanotubes in different serum and nuclease concentrations was investigated using gel electrophoresis to evaluate degradation. The nanotubes were exposed to PBS, 10% (v/v) fetal bovine serum (FBS) in PBS to mimic in vitro serum conditions, and 85% (v/v) FBS in PBS to mimic in vivo serum conditions (FIG. 3A). As a control, each of the solutions was tested in the absence of the ssDNA-amphiphile nanotubes to ensure that all signal observed originated from the nanotubes. After incubations at 37° C. for 24 hours, it was found that there no change in the electrophoretic mobility of the nanotubes when incubated with 10% FBS, suggesting no change to the nanotube structure. At 85% FBS concentration, the nanotubes showed a decrease in their electrophoretic mobility, possibly as a result of adsorption of serum proteins onto the surface of the nanotubes. However, no degradation products were observed after incubation with any serum solutions as indicated by the lack of distinct bands with higher mobility than the control sample. The nanotubes were also tested for their stability after exposure to varying concentrations of endonuclease DNase I and exonuclease III (FIG. 3A). After incubation with nuclease concentrations between 0-5 U/mL for 24 hours at 37° C., it was found that there was no degradation of the ssDNA nanotubes when exposed to either DNase I or exonuclease III, which is promising for their in vivo use. The average activity of circulating DNase I in healthy human patients is 0.356±0.410 U/mL, while the circulating activity of DNase I in human GBM patients is 0.045±0.007 U/mL. Therefore, even at much higher physiologically relevant concentrations of DNase, the ssDNA-amphiphile nanotubes show no degradation. No degradation was also observed for the exonuclease III, as the 3′-terminus of the amphiphiles is conjugated to the dialkyl tail, preventing exonuclease III from binding to the amphiphile. Degradation of the amphiphiles due to 5′ exonucleases was not investigated, as it has been shown that 5′ exonucleases from serum do not play a significant role in DNA breakdown even when the 5′-terminus is exposed. The high local concentration of the ssDNA headgroups in the self-assembled nanotubes likely prevents interaction with DNase I thus preventing degradation by internal cleavage.

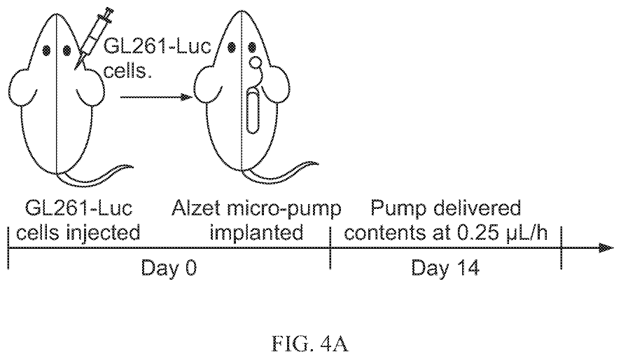

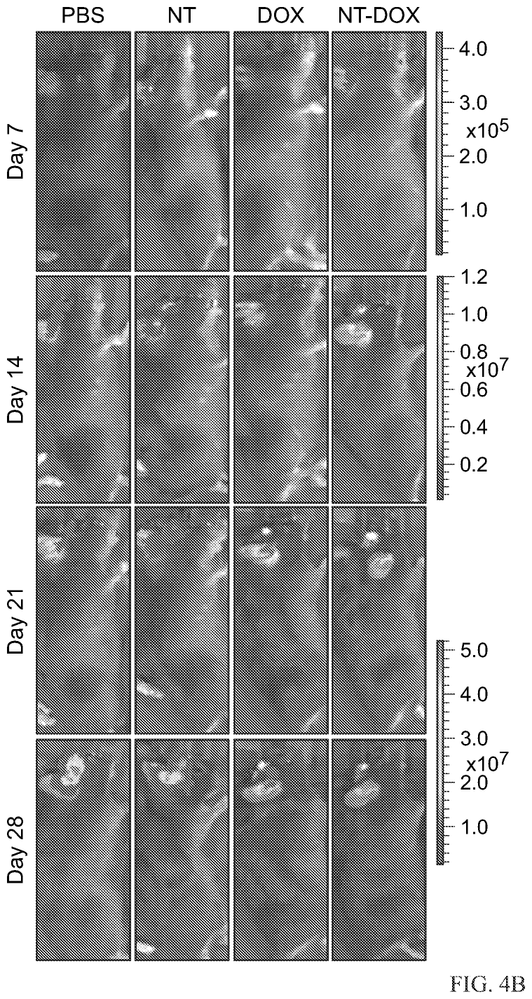

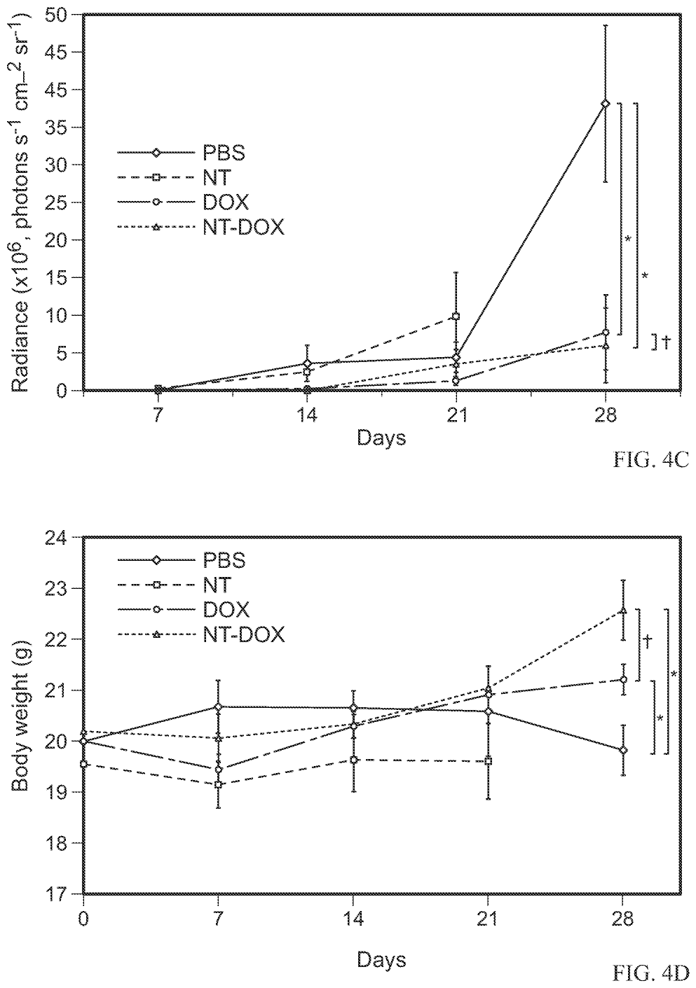

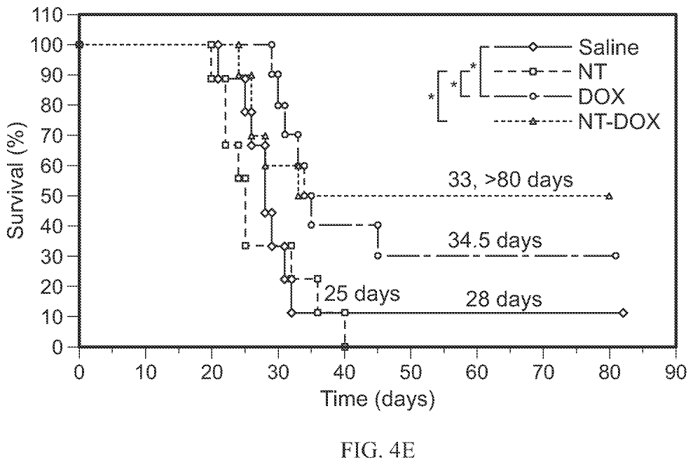

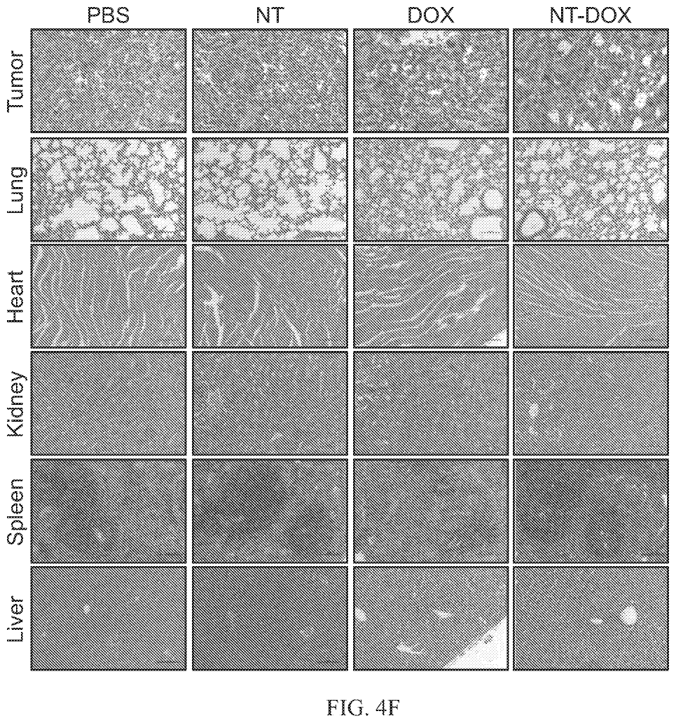

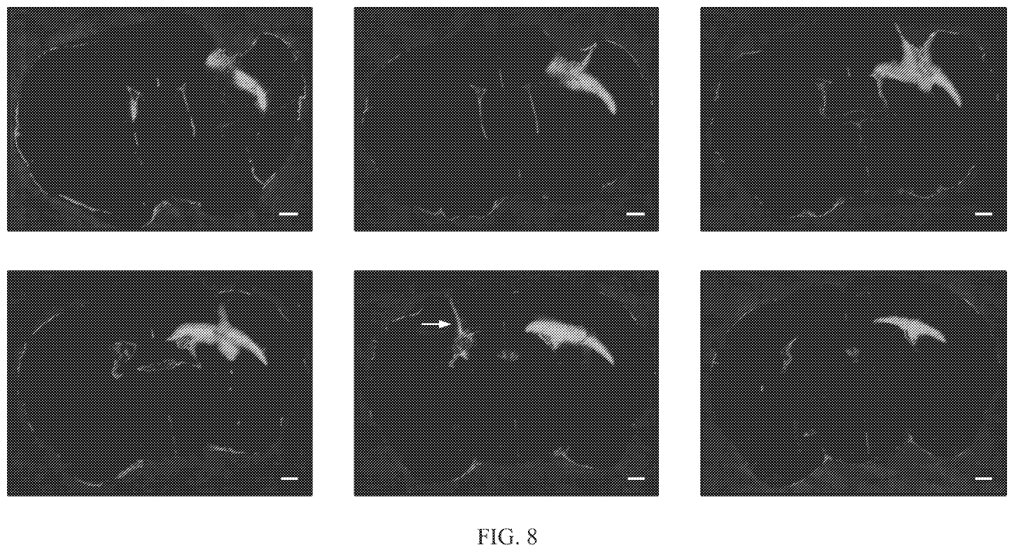

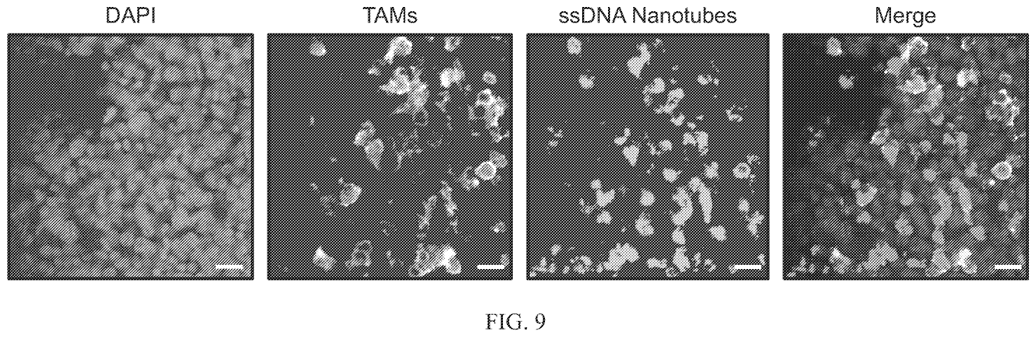

After observing strong cell internalization of the nanotubes after incubation with GL261 cells in vitro and verifying their stability, the retention of nanotubes in a more clinically-applicable system was tested by directly injecting intracranially nanotubes into an orthotopic GBM mouse model. GL261 tumors were grown in the right hemisphere of mouse brains and IRDye 800CW-labeled nanotubes were intracranially injected into both the tumor right hemisphere and healthy left hemisphere of the brain (FIG. 3B). The mice were euthanized at different time points, their brains were excised, and imaged for near infrared (NIR) fluorescence. A GL261—tumor bearing mouse did not receive any nanotube injections as a control. The average ratio of radiant efficiency between the right tumor hemisphere and left normal hemisphere was 2.25+0.07 (n=3), while the ratio for the control mouse was 1.02. The excised brains were then sectioned and stained for nuclei and glial fibrillary acidic protein (GFAP). Observed differences in NIR fluorescence between normal and tumor hemispheres was a result of differential retention of the nanotubes by the two regions despite both sides receiving an equivalent volume of IRDye 800CW-labeled nanotubes. Additional imaging of brain slices showed that this observation was consistent throughout different brain sections (FIG. 8), indicating that only the tumor hemisphere retained the ssDNA-amphiphile nanotubes. To examine the type of cells that were targeted by the ssDNA nanotubes in the tumor, fluorescently-labelled nanotubes were injected intracranially into mice bearing GL261 tumors, where the GL261 cells were either unlabeled or were expressing green fluorescent protein (GFP). Results showed that 3 hours post nanotube injection, the nanotubes were uptaken by GL261 cells (FIG. 3C and FIG. 4) and tumor associated macrophages (FIG. 9).

Treatment of GBM with Nanotubes Intercalating DOX

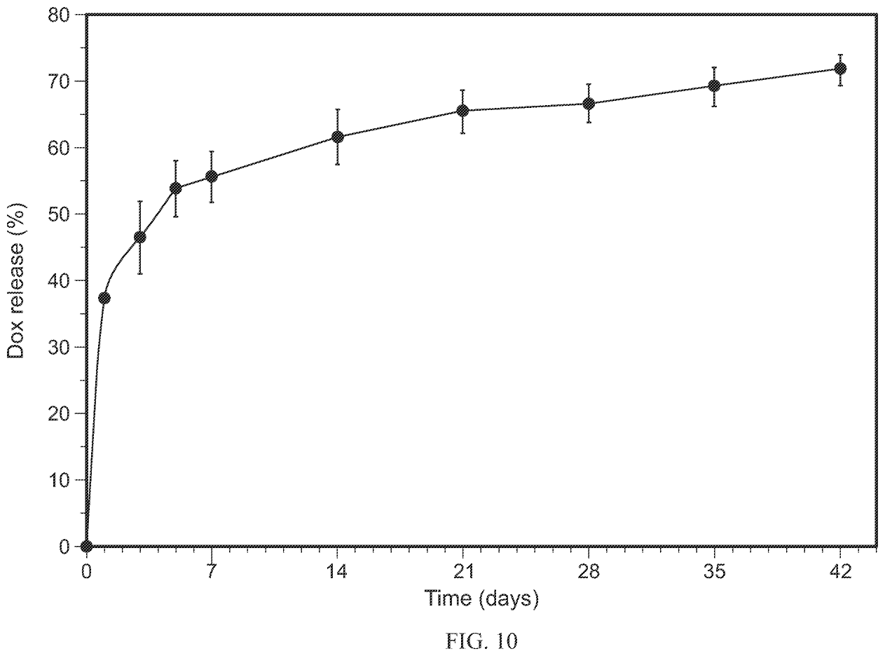

The nanotubes were used further to examine the ability of these nanoparticles to deliver a therapeutic load, such as DOX, to the GL261 cells. DOX has been shown to intercalate into the double-stranded region of ssDNA stem-loop or G-quadruplex structures, thus forming physical complexes with the ssDNA sequences through noncovalent intercalations (Manet et al., Physical Chemistry Chemical Physics, 13:540-551 (2011); and Kuang et al., Bioeng. Transl. Med., (2020)). The retention of DOX by the nanotubes was investigated by dialyzing a sample of the nanotubes that intercalated DOX against PBS at 37° C. for 6 weeks. PBS was used as a dialysis medium, both inside and outside the dialysis membrane, because it closely mimics the salt concentration of cell media and serum. As shown in FIG. 10, there is an initial burst release in the first day, where 37±1% of DOX has released, followed by a slower sustained release, with 56±4% of DOX released after 1 week, and 72±2% after 6 weeks. The effect of nanotubes (NT), DOX and DOX intercalated in the nanotubes (NT-DOX) on the viability of the GL261 cells was also assessed (FIG. 11). The empty ssDNA nanotubes were shown to have no effect on cell viability. However, there was a significant improvement in delivering DOX through the nanotubes (31±6%) compared to free DOX (47±6%), so NT-DOX was shown to be more cytotoxic to GL261 cells.

Based on the in vitro results, the nanotubes as a delivery vehicle for DOX was evaluated in an orthotopic GL261 mouse model. On the day of the surgery, 3×10⁴ viable GL261-Luc cells (GL261 cells expressing luciferase) were injected to the right side of the brain across 3 sites. Immediately following intracranial injection of the cells, a micro-osmotic pump was implanted subcutaneously and the cannula, connected to the pump through a catheter, was lowered into the brain though the same burr hole used to inject the cells (FIG. 4A). The pump delivered different treatments in about 14 days at a rate of 0.25 μL/hour. The pumps were loaded with either PBS, 70 μM of DOX (0.2 mg DOX/Kg mouse), nanotubes (NT) at 95 μM of ssDNA-amphiphiles, or NT-DOX at the same concentrations of DOX and amphiphiles. Mice were imaged weekly for 4 weeks (FIG. 4B). After 28 days from the day of surgery, analysis of the bioluminescence signal from the brain showed that the mice that received PBS had a significant increase in the size of the tumor compared to mice that received DOX or NT-DOX (FIG. 4C). Data are not reported for the NT group, as only 3 mice were alive on day 28. Mice treated with DOX or NT-DOX demonstrated an 80% and 84% decrease in tumor signal respectively. There was no significant statistical difference in the tumor signal for mice that received DOX and NT-DOX. The changes in bodyweight were also measured for all groups (FIG. 4D) and it was found that mice treated with PBS lost on average 1% body weight after 28 days (not statistically significant), whereas mice treated with DOX gained 6% body weight and mice treated with NT-DOX gained on average 12% body weight during the same time (both changes were statistically significant). In addition, on day 28 the difference between the weight of mice treated with PBS and DOX or NT-DOX was statistically significant, while the difference between the weight of mice treated with DOX and NT-DOX was not statistically significant. The effective inhibition of tumor growth by the DOX and NT-DOX treatments correlated with an increase in animal survival (FIG. 4E). The median survival time of mice was 28 days for the PBS group, with 1 mouse out of 9 surviving for more than 82 days. The median survival time of mice that were treated with NT was 25 days (difference not statistically significant with PBS). In contrast, mice receiving DOX had a significant increase in their median survival (34.5 days, 3 out 10 mice survived for more than 82 days). For the NT-DOX group, the survival curve is horizontal at 50% survival. 50% of mice (5 mice) had a median survival time of 33 days and 50% (5 mice) survived for more than 82 days (although differences between the NT-DOX and PBS or DOX groups were not statistically significant).

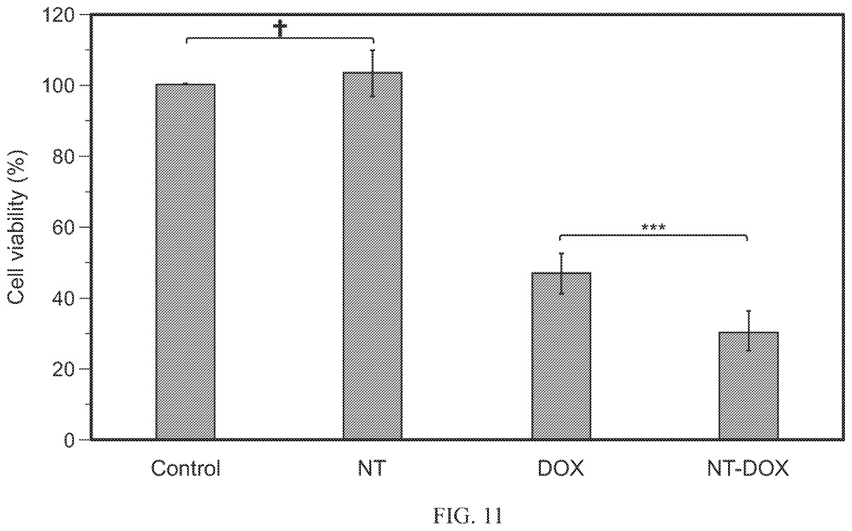

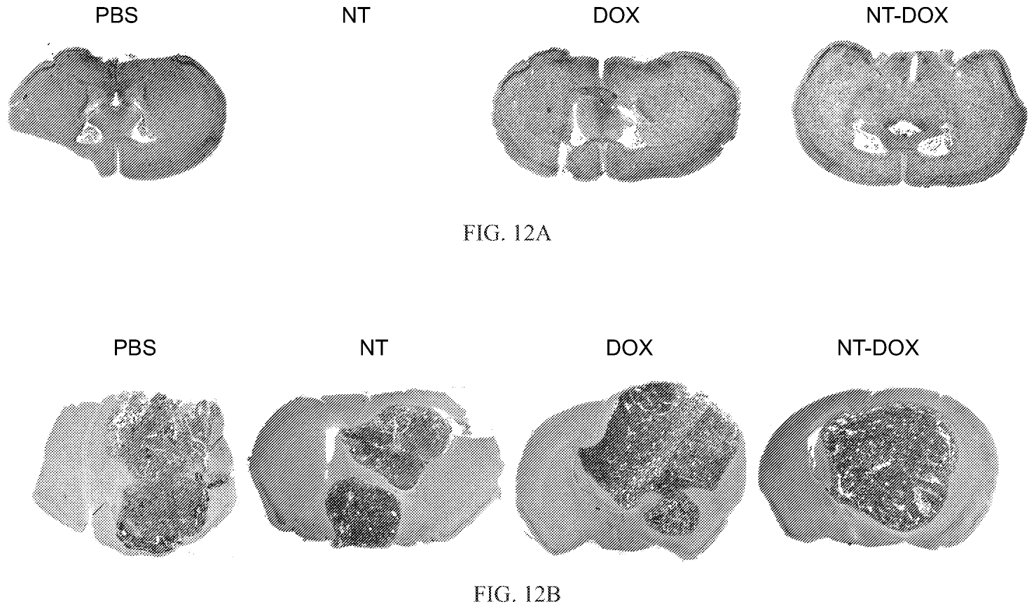

The advantage of the NT-DOX group compared to DOX was shown after histological examination of different organs. Histological analysis of the brain, liver, spleen, lungs, kidneys and heart tissues of mice was performed at the end of the experiment. From the mice that were examined and were long-term survivors at the end of the experiment on day 82, no tumors were observed in their brains (FIG. 12A). For the rest of the mice, invasive tumors with anaplastic features were present (FIG. 12B and FIG. 9F). The tumor cells had well delimited margins. In all cases the tumors were infiltrative, focally necrotic (ranging from <5% to 10%) and consisted of sheets of cells separated by a mucinous or a fine fibrovascular stroma. The tumor cells showed marked anaplasia, anisokaryosis and mitoses, with frequent atypia. Many multinucleate giant tumor cells were also present. There were few and inconsistent differences between the morphology of the tumors in the different groups. Compared to the PBS control, lung, kidney and heart tissues of mice treated with DOX, NT or NT-DOX showed no significant findings (FIG. 4F). However, the spleen of mice treated with DOX showed diffuse, moderate to marked depletion of the spleen white pulp associated with zonal lymphocytic apoptosis and necrosis, and moderate, diffuse hemosiderosis was observed in the red pulp (FIG. 4F). In addition, the liver exhibited multifocal areas of hepatocyte degeneration and necrosis occasionally associated with neutrophilic and lymphohistiocytic infiltrate, multifocal individual hepatocyte necrosis/apoptosis and diffuse glycogen depletion (FIG. 4F). DOX has been shown to trigger splenic marginal depletion of the spleen white pulp and liver focal necrosis. In contrast, there were no significant findings in spleen and liver tissues of mice treated with NT or NT-DOX, thus demonstrating no microscopic toxicity to mice.

### Biodistribution of Systemically Delivered Nanotubes in Orthotopic GL261-Tumor Bearing Mice

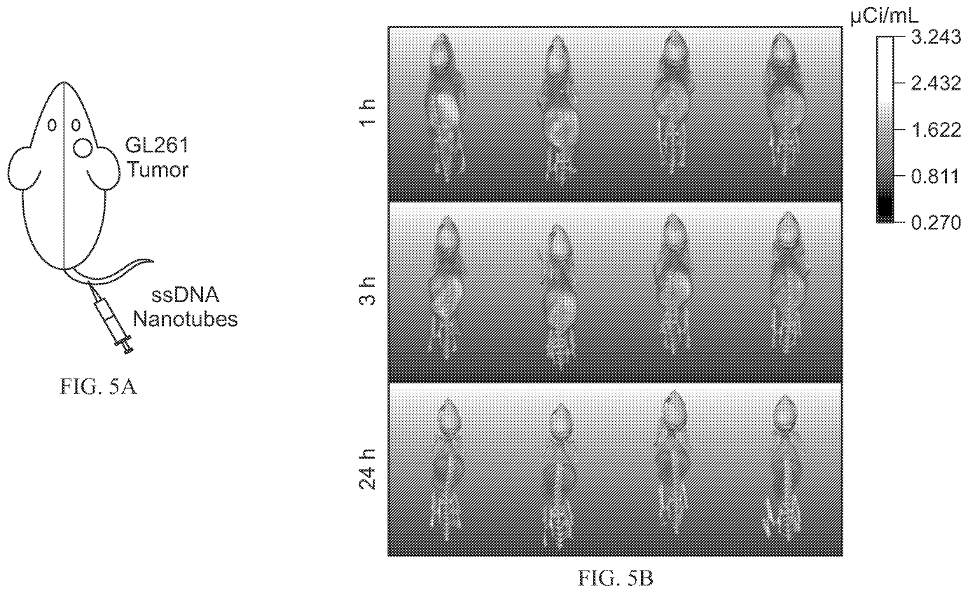

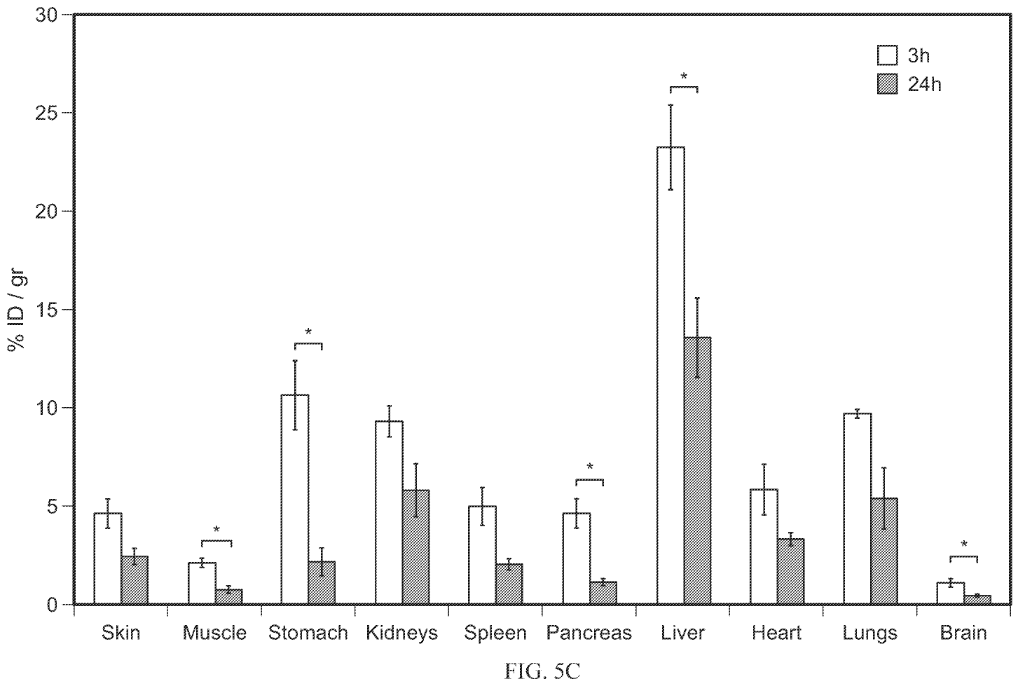

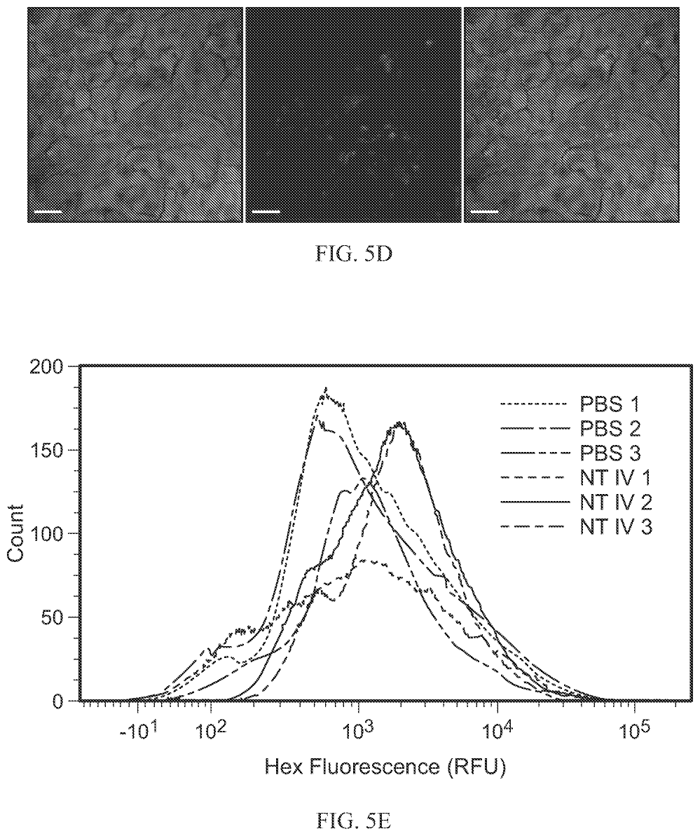

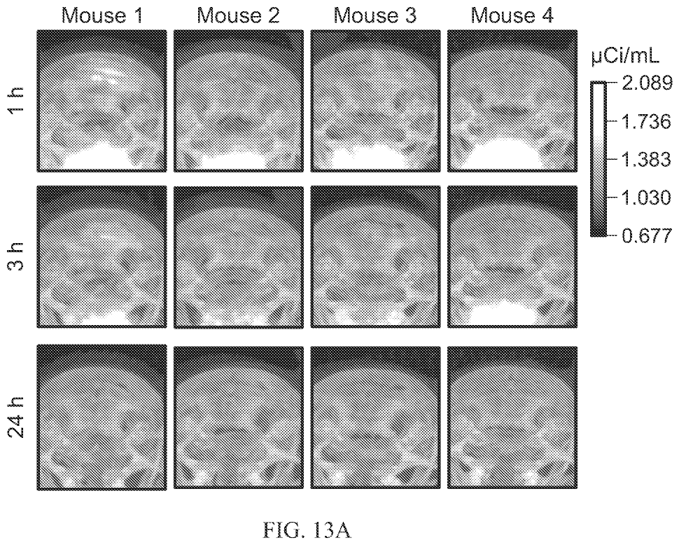

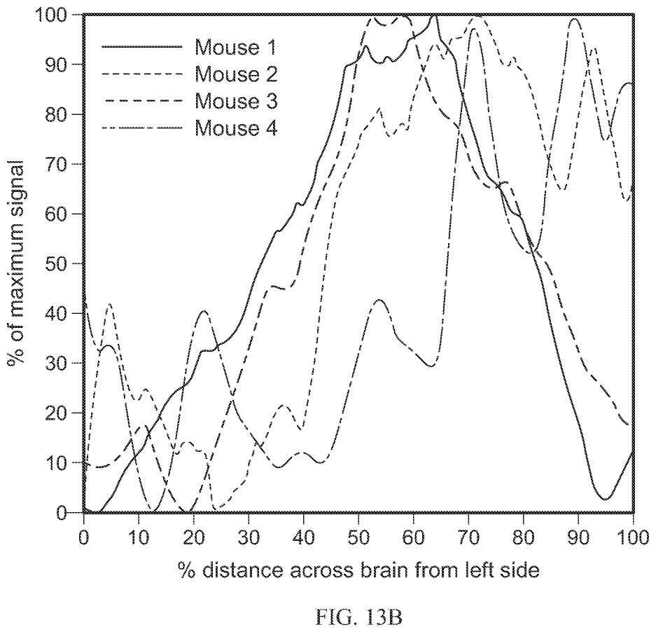

To explore the nanotube biodistribution and if they can pass the BBTB after tail vein injection, nanotubes were labeled with 1,4,7,10-tetraazacyclododecane-1,4,7,10-tetraacetic acid (DOTA) to chelate ⁶⁴Cu for biodistribution experiments. The DOTA-labeled amphiphiles were present in approximately 1850× molar excess of ⁶⁴Cu, ensuring that all available copper was chelated by the nanotubes. Mice bearing orthotopic GL261 tumors on the right brain hemisphere were injected with nanotubes through their lateral tail vein (FIG. 5A) and imaged with micropositron emission tomography/computed tomography (μPET/CT) at 1, 3, and 24 hours post injection (FIG. 5B). FIG. 4B shows that the liver is the organ with the highest radioactivity at all time points and some activity is also shown on the head of the mice. To accurately evaluate biodistribution of the nanotubes, mice were euthanized at either 3 or 24 hours. Excised organs were weighed and measured for radioactivity to evaluate nanotube biodistribution at each time point. The activity of each organ was adjusted for the half-life decay of ⁶⁴Cu and expressed as percentage of injected dose per gram of tissue (% ID/gr), shown in FIG. 5C. All organs measured showed a decrease in radioactivity between 3 and 24 hours (FIG. 5C), and the organ with the highest accumulation at both time points was the liver, consistent with the blood clearance profiles of many types of nanoparticles. Brain accumulation at 3 hours was 1.08±0.16% ID/gr (0.54±0.09% ID) and at 24 hours was 0.40±0.07% ID/gr (0.19±0.03% ID). To evaluate if the nanotubes were on the tumor-bearing brain hemisphere after systemic administration, maximum-intensity projections of the heads of each mouse from the μPET/CT images at 1, 3 and 24 hour time points were examined (FIG. 13A). These images are tail-view projections, viewing the head of the mouse looking from the tail, through the head, and out through the nose of the mouse. μPET profile intensity plots relative to the left edge of the mouse cranium suggest that there may be preferential nanotube accumulation in the right hemisphere of the brain, the hemisphere which received the GL261 cells (FIG. 13B). To further elucidate if the nanotubes could cross the BBTB, the colocalization of GL261 cells and fluorescently-labelled nanotubes was evaluated after intravenous injection of the nanotubes into mice bearing orthotopic tumors of GL261 cells expressing GFP at different times.

Tumor tissues were removed and evaluated via confocal microscopy and flow cytometry. Confocal images showed that 3 hours post nanotube injection, the nanotubes were uptaken by GL261 cells (FIG. 5D). In addition, 6 hours post nanotube injection the nanotubes were also associated with GL261 cells as evident by the extra peak on the flow cytometry data (FIG. 5E), that is attributed to the presence of the nanotubes in the tumor samples. Taken together these data suggest that the nanotubes can cross the BBTB after intravenous injection and associate with the GBM cells.

Collectively, these results demonstrate the promising therapeutic advantages of ssDNA nanotubes intercalated with DOX as an anticancer agent, as it increased the number of long-term survivors and minimized toxicity to healthy organs. In addition, the ability of the nanotubes to target tumors by binding to scavenger receptors that are over-expressed in GBM and other cancers, internalize through micropinocytosis that is highly activated in GBM cells compared to normal cells, stability in nucleases and serum, ability to cross the BBTB and accumulated in the brain at higher amounts compared to other nanoparticles, preferential retention by tumors compared to normal brain, and ability to load and deliver chemotherapy drugs such as DOX, nanotubes formed through the self-assembly of ssDNA-amphiphiles may have potential for translation as a drug delivery vehicle to GBM tumors.

### Methods

### Materials

All materials were purchased from Sigma Aldrich and used without further purification or modification unless otherwise stated. Buffers include high performance liquid chromatography (HPLC) buffer A (100 mM hexafluoroisopropanol and 15 mM triethylamine in Milli-Q water), HPLC buffer B (100 mM hexafluoroisopropanol and 15 mM triethylamine in methanol), TEAA buffer (50% molar basis triethylamine, 50% molar basis glacial acetic acid, pH=7.0), Cu-TBTA (10 mM Copper (II)-Tris[(1-benzyl-1H-1,2,3-triazol-4-yl)methyl]amine) in 55% dimethyl sulfoxide (DMSO), 45% Milli-Q water), 1× phosphate buffered saline (PBS) (137 mM sodium chloride, 2.7 mM potassium chloride, 10 mM disodium phosphate, 1.8 mM monopotassium phosphate in Milli-Q water, pH=7.4), and 1× TAE (40 mM tris(hydroxymethyl)aminomethane, 20 mM acetic acid, and 1 mM ethylenediaminetetraacetic acid).

Synthesis of ssDNA-Amphiphiles

ssDNA sequences were purchased from Integrated DNA Technologies (Coralville, IA) with a 3′-amino modifier and an optional 5′ HEX (538/555 nm ex/em) or hexynyl (alkyne) group. A 10 nt sequence (5′-CTCTTGGGGG-AmMO-3′; SEQ ID NO:1) was used in this study. ssDNA was precipitated in Milli-Q water using 100 mM cetyl trimethylammonium bromide (CTAB) and centrifuged for 15 minutes at 16,100 g, followed by removal of the liquid and drying of the precipitate under an airstream to remove any excess water. The dried precipitated ssDNA was then resuspended in 90%/10% (v/v) mixture of dimethylformamide (DMF) and DMSO at 500 μM. The C₁₆ dialkyl tail with the C₁₂ hydrocarbon spacer was synthesized as described elsewhere (Waybrant et al., Langmuir, 30:7465-7474, (2014)), added in 10 times molar excess, and reacted for 16 hours at 65° C. The solution was concentrated by drying in a vacuum oven until approximately 50 μL in volume. The reaction product with the ssDNA-amphiphile and unreacted ssDNA was precipitated by a lithium perchlorate precipitation, where 1 mL of lithium perchlorate in acetone (2.5% w/v) was added and the solution was mixed until homogeneous, followed by the addition of 100 μL of Milli-Q water and placed in a −20° C. freezer for 15 minutes. The precipitate was centrifuged for 15 minutes at 16,100 g and rehydrated with 1 mL of Milli-Q water and filtered through a 0.45 μm polyether sulfone filter (GE Healthcare, Chicago, IL). The filtered ssDNA-amphiphiles were separated from unreacted ssDNA using HPLC with HPLC buffer A and HPLC buffer B over 30 minutes. ssDNA-amphiphiles were then dried under an air stream to approximately 150 μL, precipitated with 1 mL of lithium perchlorate in acetone to remove HPLC buffer components and rehydrated at 500 μM in Milli-Q water for storage at −20° C. DOTA-labeled ssDNA-amphiphiles were synthesized as described elsewhere (Harris et al., Nanomedicine: NBM, 14:85-96 (2018)). The molecular weight of ssDNA-amphiphiles was verified by liquid chromatography-mass spectrometry (LC-MS). For the synthesis of RDye 800CW-labeled amphiphiles, ssDNA-amphiphiles with a 5′-alkyne modification were mixed in 50% Milli-Q water and 50% DMSO to a final concentration of 100 μM. TEAA buffer was added to a concentration of 200 mM, Cu-TBTA buffer was added to a concentration of 1 mM, ascorbic acid was added to a concentration of 2 mM, and TRDye 800CW Azide (778/794 nm ex/em) (Licor, Lincoln, NE) was added in five times molar excess of the ssDNA-amphiphiles. The solution was mixed and left overnight in the dark at room temperature, followed by a lithium perchlorate in acetone precipitation to remove excess buffer components. The dye-labeled ssDNA-amphiphiles were rehydrated at 500 μM in Milli-Q water for storage at −20° C.

### Circular Dichroism (CD)

ssDNA-amphiphiles solutions or pure ssDNA were diluted to 35 μM in Milli-Q water or PBS and transferred to a 0.1 cm path length cuvette. CD spectra from 320-200 nm were collected using an AVIV 420 CD Spectrometer using a 1 nm step size with an averaging time of 5 seconds and a settling time of 0.333 seconds. The background spectrum from the Milli-Q water or PBS was subtracted and the raw ellipticity values were converted to molar ellipticity. Data were smoothed using the Sovitsky Golay Filter function (sgolayfilt) on Matlab using an order of 3 and a frame length of 11.

### Nanotube Preparation

ssDNA nanotubes containing different amphiphiles (unlabeled mixed with fluorescently-labeled or DOTA-labeled) were created by combining the desired amphiphiles at the correct ratio in Milli-Q water. One volume equivalent of DMSO was added to the mixtures so the final DMSO concentration was 50% (v/v). The mixtures were then stirred for 4 hours, during which Milli-Q water was slowly added to the mixtures until the final DMSO concentration at 4 hours was 10% (v/v). Mixtures were dialyzed overnight using a Tube-O-DIALYZER Medi 1K MWCO dialysis membrane (G-Biosciences, St. Louis, MO) to remove excess DMSO and dried under an air stream to 500 μM to prepare for nanoparticle separation. Nanotubes were separated from micelles using size exclusion chromatography on an Akta fast protein liquid chromatography (FPLC) (Amersham Biosciences, Piscataway, NJ). A C10/20 Column (GE Healthcare, Chicago, IL) loaded with Sepharose CL-4B chromatography matrix was used to separate the nanoparticles. 500 μM of ssDNA-amphiphile mixtures were loaded at 500 μL per run onto the column and separated using Milli-Q water as a buffer. Fractions were collected based on UV absorbance of the eluent, dried under an airstream to 500 μM.

### Cryogenic Transmission Electron Microscopy (Cryo-TEM)

Lacey Formvar/Carbon 200 mesh copper grids were purchased from Ted Pella (Redding, CA) and glow-discharged for 1 minute to make the grids more hydrophilic. 4.5 μL of 500 μM ssDNA-amphiphiles in Milli-Q water were deposited onto the grid and vitrified in liquid ethane using a Vitrobot (Vitrobot parameters: 5 second blot time, 3 second wait time, 3 second relax time, 0 offset, 95% humidity, 25° C.). The grids were transferred to and kept under liquid nitrogen until imaged on a Tecnai G2 Spirit TWIN 20-120 kV/LaB₆ TEM operated at an accelerating voltage of 120 kV using an Eagle 2k CCD camera.

### Cell Culture

GL261 mouse GBM cells (originally from NIH) or C8-D1A normal mouse astrocytes (ATCC, Manassas, VA) were cultured at 37° C. and 5% CO₂ using Dulbecco's Modified Eagle Medium (DMEM) (Thermo Fisher Scientific, Rockford, IL) supplemented with 10% (v/v) fetal bovine serum (FBS) (Thermo Fisher Scientific, Rockford, IL) and 100 units/mL penicillin, 0.1 mg/mL streptomycin. Cells were passaged when they reached 80% confluence by treatment with TrypLE Express Cell dissociation agent (Thermo Fisher Scientific, Rockford, IL).

### Nanotube Cell Internalization and Organelle Colocalization Via Confocal Microscopy

ssDNA-amphiphile nanotubes containing 20 mol % HEX-labeled ssDNA-amphiphiles were prepared at 250 μM in PBS. 200,000 GL261 or C8-D1A cells were deposited onto glass coverslips within wells of a 24-well plate and allowed to adhere and proliferate for 24 hours. The next day, media was replaced with 500 μL of fresh media, and nanoparticles were added to a final concentration of 12.5 μM. After 24 hours, the media containing nanotubes was removed and the cells were washed once with PBS. The cells were then stained simultaneously for their nuclei and membranes using Hoechst 33342 (Thermo Fisher Scientific, Rockford, IL) at 0.92 μg/mL and Wheat Germ Agglutinin AlexaFluor647 (Thermo Fisher Scientific, Rockford, IL) at 5.0 μg/mL respectively for 7 minutes at 37° C. The cells were then washed once with PBS and fixed using 4% paraformaldehyde in PBS for 10 minutes at room temperature, and then washed twice with PBS to remove any remaining paraformaldehyde. Cells were mounted onto glass slides using Prolong Diamond Antifade Mountant (Thermo Fisher Scientific, Rockford, IL) and imaged with an Olympus FluoView FV1000 BX2 Upright Confocal Microscope. Image analysis was performed using ImageJ software. Organelle colocalization was performed in the same manner, but the media replenishment after 24 hours used 1 mL instead of 500 μL. Early endosomes were stained by adding 10 μL of CellLight Early Endosomes-GFP Bacman 2.0 (Thermo Fisher Scientific, Rockford, TL) for a final concentration of 10 particles per well. The CellLight solution was added at the same time as the HEX-labeled ssDNA-amphiphiles, which had a final concentration of 12.5 μM. 2 hours prior to the completion of the 24 hours incubation, Lysotracker Deep Red (Thermo Fisher Scientific, Rockford, IL) was added to the wells at a final concentration of 200 nM. At the end of the 24 hours incubation, the media containing nanoparticles was removed and the cells were washed once with PBS. The nuclei were then stained using Hoechst 33342 at 0.92 μg/mL for 10 minutes at 37° C., washed once with PBS, fixed with 4% paraformaldehyde in PBS for 10 minutes at room temperature, washed twice with PBS, and mounted onto glass slides using Prolong Diamond Antifade Mountant. The cells were then imaged with an Olympus FluoView FV1000 BX2 Upright Confocal Microscope, with image analysis performed in ImageJ software. Manders coefficients were calculated by drawing a region of interest around each cell excluding the nuclei and using ImageJ's Coloc2 plugin. The coefficients reported here are the percent of nanoparticle signal that overlapped with either early endosomes or lysosomes. Percent free nanoparticle signal for each cell was calculated by using ImageJ's Measure tool to sum the total pixel intensity of nanoparticle signal that did not co-occur with either endosomes or lysosomes and dividing by the total nanoparticle signal pixel intensity.

### Inhibition of Endocytosis

200,000 GL261 cells were plated per well in 12-well plates and incubated at 37° C. and 5% CO₂ overnight. The following day, media was replaced with fresh media, and inhibitor stock solutions were diluted and delivered to the cells at targeted concentrations: 1.25 μg/mL cytochalasin D (CytD), 10 μM latrunculin B (LatB), 15 μg/mL nocodazole, 5 μg/mL fillipin, 2.5 μg/mL nystatin, 1.32 mg/mL methyl-β-cyclodextrin (MPCD), 2.5 μg/mL chlorpromazine (CPZ), 12.5 μg/mL dynasore, 0.2 μg/mL pertussis toxin (PTX), 2 μg/mL cholera toxin (CTX), 30 μg/mL 5-(N,N-dimethyl)-amiloride hydrochloride (DMA), and 500 μg/mL fucoidan. Cells with no inhibitor were used as positive controls. Plates were gently shaken by hand to ensure even distribution of inhibitors, and placed in the incubator for 30 minutes. After that, 5 nmol of nanotubes containing 20 mol % HEX-labeled ssDNA-amphiphiles were added to each well, plates were gently shaken by hand and placed in the incubator for 3 hours. Cells were washed twice with 1 mL PBS, trypsin-EDTA at 37° C. was added to each well, the contents of each well were individually mixed via up and down pipetting and transferred to 1.5 mL centrifuge tubes. Cells were washed twice with PBS, reconstituted in 500 μL PBS and transferred to flow cytometry tubes. 5 μL of 10 μg/mL propidium iodide (PI) solution was added to each tube and vortexed. The cells were run on a BD FACSCanto flow cytometer and were examined for PI and HEX fluorescence via excitation at 488 nm with a 585/42 filter. Cells were gated by PI staining to select for live cells, and by scattering (FSC-A and SSC-A) to select for single cells. To evaluate cytotoxicity of inhibitors, 10,000 GL261 cells were plated in 96-well plates and incubated overnight in a 37° C. 5% C02 incubator. The next day, media was replaced with fresh formulated media, and inhibitor stock solutions were diluted and delivered to the cells at concentrations mentioned above. Plates were gently shaken by hand and placed in the incubator for 3 hours. Cell viability was measured using CellTiter-Glo 2.0 assay (Promega, Madison, WI) following the manufacturer's protocol. Luminescence was recorded using a Synergy H1 microplate reader (Biotek, Winooski, VT), and cell viability was normalized to untreated cells.

### Nanotube Serum and Nucleases Stability Evaluated Via Gel Electrophoresis

Nanotubes containing 20 mol % HEX-labeled ssDNA-amphiphiles were prepared at 250 μM in Milli-Q water on an amphiphile basis. For the serum stability experiment, nanotubes were mixed into three separate conditions using 2.5 μL of nanotubes and 47.5 μL of solution, for 50 μL total volume of mixture and 12.5 μM final ssDNA-amphiphile concentration. The three solutions used were: 5 μL of 10× PBS with 42.5 μL Milli-Q water as a control, 5 μL 10× PBS with 5 μL FBS with 37.5 μL of Milli-Q water (10% v/v FBS) to mimic in vitro conditions, and 5 μL 10× PBS with 42.5 μL FBS (85% v/v FBS) to mimic in vivo conditions. For the nucleases stability experiment, nanotubes were mixed with DNase I and exonuclease III (Thermo Fisher Scientific, Rockford, IL) using 2.5 μL of nanotubes and 47.5 μL of solution for 50 μL total solution and 12.5 μM final ssDNA-amphiphile concentration. The 47.5 μL solutions of nucleases contained 5 μL of the 10× reaction buffer provided by each kit to create a final concentration with the ssDNA-amphiphiles of 1× reaction buffer. Nuclease concentrations were tested between 0 and 5 U/mL final concentration. All nanotube-serum and nanotube-nucleases solutions were incubated at 37° C. for 24 hours and were run on 2% agarose gels (2% agarose in 1× TAE buffer) at 120 V for 35 minutes, and imaged using a ChemiDoc MP Imaging System (Bio-Rad, Hercules, CA).

### Animals

C57BL/6J mice were purchased from Jackson Laboratories (Bar Harbor, ME). Studies were in accordance with the NIH Guide for the Care and Use of Laboratory Animals.

### Bilateral Intracranial (IC) Injections of Nanotubes to Orthotopic GL261 Tumor-Bearing Mice and Whole Brain Imaging

Four mice were placed into deep anesthesia using an intraperitoneal injection of 100 mg/kg ketamine (Vedco, St. Joseph, MO) and 10 mg/kg xylazine (Akorn Animal Health, Lake Forest, IL). Buprenorphine (0.03 mg/mL intramuscular) was administered, the mouse head was sterilized, and a 1 cm incision was made along the scalp. 30,000 GL261 cells in sterile PBS were implanted into the right-side striatum of the mice using a murine stereotaxic system (Stoelting Co, Wood Dale, IL). Nanotubes containing 20 mol % IRDye 800CW-labeled ssDNA-amphiphiles were prepared in PBS. Three of the mice were anesthetized with ketamine (100 mg/kg) and xylazine (10 mg/kg) and 2 μL of the nanotube solution (2 nmol of ssDNA-amphiphiles) was injected bilaterally into both the left (normal side) and right (tumor side) striatum, 14 days after tumor cell implantation. At 45 to 105 minutes after nanotube injections, mice were decapitated, and brains were taken and fixed in 4% paraformaldehyde overnight. Mouse brains were imaged using an In Vivo Imaging System (IVIS) with 780/820 nm excitation emission settings. Following direct imaging, the mouse brains were dehydrated in 30% sucrose in PBS and embedded with Tissue-Tek optimal cutting temperature (OCT.) cryo-compound (Sacura, Torrance, CA). The brains were then frozen at −80° C., 10 μm sections were cut using a Leica cryostat (Wetzlar, Germany), mounted onto charged Superfrost Plus glass slides (Thermo Fisher Scientific, Rockford, IL), and stored at −20° C. until staining. The tissue sections were incubated with polyclonal antibodies against glial fibrillary acidic protein (GFAP) (Lifespan Biosciences, Seattle WA) diluted 1:500 in PBS with 1% tween and 5% donkey serum in a humidified chamber at 4° C. overnight. The sections were then incubated with AlexaFluor-488 secondary antibody diluted 1:750 (R&D Systems, Minneapolis MN) for 1 hour at room temperature, followed by staining with DAPI for 10 minutes at room temperature. Mounted slices were imaged in three fluorescent channels using a Nikon Eclipse TE2000-U inverted wide-field fluorescent microscope. Image analysis was performed using ImageJ software.

Evaluation of Nanotube Association with Tumors after Intracranial (IC) or Intravenous (IV) Injections to Orthotopic GL261 Tumor-Bearing Mice To establish the xenograft, GFP-labeled GL261 cells were dissociated into single-cell suspensions and stereotactically injected into the brains of 12 week old mice (50,000 cells per injection). 3 weeks post tumor cell injection, 20 mol % HEX-labeled nanotubes were administered to the mice via IC or IV injection. For the IC route, 1 nmole of ssDNA-amphiphiles dissolved in 2 μL PBS, or 2 μL PBS were injected for each mouse. For the IV route, 30 nmole of ssDNA-amphiphiles dissolved in 200 μL PBS, or 200 μL PBS were injected for each mouse. The mice were sacrificed 3 hours post IC injection, or 6 hours post IV injection of nanotubes and tumor tissues were removed. For confocal, resected tumor tissues were immediately snap-freezed and later cryo-sectioned axially into 30 μm slices using a Lecia CM 1905 cryostat. Mounted slices were imaged on a Zeiss LSM700 confocal microscope. Settings were optimized to avoid background fluorescence using untreated brain slices. Zen software was used to process the obtained images.

Tumors evaluated via flow cytometry were rinsed with PBS, transferred to a petri dish, and mechanically disaggregated to slurry consistency with fine scissors. 1-2 mL DMEM-F12 medium (Thermofisher Scientific, Rockford, IL) was added to tumor slurry, followed by repeatedly pipetting up and down with a 1 mL pipette tip to break down visible aggregates. Dissociated tumor samples were then pipetted up and down with a 200 μL pipette tip, transferred to a 15 mL tube and centrifuged at 300×g for 5 minutes. Cell pellets were resuspended in 2 mL DMEM-F12 medium and filtered through a cell strainer (70 μm). Cells were diluted with PBS and fixed with 4% paraformaldehyde. Fixed samples in PBS were subsequently run on a BD FACSCanto flow cytometer.

For evaluation of nanotube association with TAMs, 8 week old mice were IC inoculated with 100,000 GL261 cells. On day 14 after tumor inoculation, 20 mol % HEX-labeled nanotubes (1 nmole of ssDNA-amphiphiles dissolved in 2 μL PBS) were administered intratumorally to a depth of 2 mm into the original burr hole for tumor inoculation. Brains were collected 3 hours after nanotube injection and immediately placed in 10% formalin solution overnight, followed by a daily sucrose gradient (10, 20, then 30% sucrose in PBS) to wash out the formalin. Fixed brains were then flash frozen on dry ice and cryosectioned axially into 30 μm slices using a Lecia CM 1905 cryostat. Brains were stained with DAPI to visualize cell nuclei and Ibal primary antibody at 1:200 (Wako Pure Chemical Corporation, Tokyo, Japan) to visualize macrophages. Briefly, brain slices were blocked with tris-buffered saline (Corning, Coming, NY) supplemented with 0.1% triton-X, 1% bovine serum albumin, and 5% normal goat serum (ThermoFisher, Waltham, MA) for 4 hours, followed by incubation with unconjugated primary antibodies overnight at 4° C. Then slices were washed and incubated with goat anti-rabbit 488 secondary antibody (Invitrogen, Carlsbad, CA) for 2 hours at room temperature. Finally, slices were incubated with DAPI nuclear stain for 15 minutes, mounted in fluorescence mounting media (Agilent Technologies, Santa Clara, CA), sealed and imaged using a Zeiss LSM710 confocal microscope. Settings were optimized to avoid background fluorescence using untreated brain slices. Zen software was used to process the obtained images.

### Preparation of Nanotubes Intercalating DOX (NT-DOX) and DOX Release

Doxorubicin-hydrogen chloride (DOX) dissolved in water at 1 mg/mL was combined on an equimolar basis with ssDNA-amphiphiles in water at 500 μM. DMSO was added to the solution until the final DMSO concentration was 50% (v/v). The solution was stirred for 2 hours. Over 4 additional hours, water was slowly added until the final concentration was 90% water, 10% DMSO (v/v) at the end of the 4 hour period. The mixture was dialyzed overnight in a Tube-O-DIALYZER Medi 1k MWCO dialysis membrane (G-Biosciences, St. Louis, MO) to remove the DMSO. Nanotubes intercalating DOX were separated from micelles intercalating DOX as described above under nanotube preparation. DNA concentration was calculated through the absorbance of light at 260 nm. However, DOX also absorbs light at this wavelength. Therefore, the absorbance of mixtures of ssDNA and DOX was measured at both 260 nm and 488 nm, the maximum absorbance wavelengths for DNA and DOX respectively. The extinction coefficient of the ssDNA at 260 nm was provided by IDT as 89300 cm⁻¹M⁻¹ and assumed to remain the same after the attachment of the hydrophobic tail. The extinction coefficient of the ssDNA at 488 nm was calculated by measuring the absorbance of a known amount of ssDNA at both 260 nm and 488 nm, providing an extinction coefficient at 488 nm of 135 cm⁻¹ M⁻¹. Several known concentrations of DOX were prepared by weighing out solid DOX and suspending in known volumes of Milli-Q water. The absorbance for each DOX sample was measured at both 260 nm and 488 nm, allowing for the calculation of the extinction coefficients for DOX as 14715 cm⁻¹ M⁻¹ and 10200 cm⁻¹ M⁻¹, respectively. With all four extinction coefficients and the absorbance measurements at both 260 nm and 488 nm, the concentration of ssDNA-amphiphiles and DOX was calculated by solving the two coupled linear equations. It was assumed that the absorbance of the nanotubes and DOX was additive with no interacting terms. NT-DOX mixtures (200 μL) with 75 μg/mL of DOX and 76 μM of ssDNA-amphiphiles on average in PBS were placed in a D-Tube Dialyzer Midi, MWCO 3.5 KDa.

The dialysis tube was placed in a beaker with 100 mL PBS at 37° C. At several time points during the dialysis small samples were taken out and the absorbance at 260 nm and 488 nm was measured to determine the DOX concentration.

### Cell Viability

The effect of DOX, nanotubes, and NT-DOX on cell viability was assessed using the CellTiter-Glo 2.0 assay. 10,000 GL261 cells were deposited into black 96-well tissue culture treated plates with 100 μL of media and allowed to adhere for 24 hours at 37° C. The next day, media was removed, 95 μL of new media was added and 5 μL of each test sample dissolved in Milli-Q water was added: water (control), nanotubes at 5-6.4 μM of ssDNA-amphiphiles, free DOX at 5 μg/mL, or NT-DOX at the same DOX and amphiphile concentrations. The samples were incubated with cells for 12 hours at 37° C., followed by a single wash with PBS. 100 μL of fresh media was added, and cells were incubated at 37° C. for an additional 36 hours. Cells were allowed to equilibrate to room temperature, while the CellTiter-Glo 2.0 solution was placed in a room temperature water bath. 100 μL of the CellTiter-Glo 2.0 solution was added to each well of cells simultaneously and the entire plate was placed on an orbital shaker for 2 minutes and then allowed to rest for 10 minutes. The luminescence signal of each well was measured, and the luminescence of each group was normalized to the luminescence of the untreated cells.

Intracranial Delivery of Nanotubes Intercalating DOX Via CED and Bioluminescence Imaging of Mice GL261-Luc cells were transfected to express luciferase. Glioma media consisted of DMEM high glucose and L-glutamine (Genesee Scientific 25-500), supplemented with 10% FBS, 1% penicillin-streptomycin (HyClone SV30010) and 1% MEM NEAA (Gibco 1140-050). Media was changed every other day and cells were passaged when reaching 80% confluence using TrypLE. Prior to transplantation cells were washed three times with PBS followed by trypsinization for 5 minutes at 37° C. followed by inactivation of the trypsin and centrifugation. The resulting pellet was resuspended in cold Hank's Balanced Salt Solution (HBSS; Life Technologies) for counting using a hemocytometer. The cells were centrifuged a second time and resuspended at a concentration of roughly 1×10⁴ cells per μL of cold HBSS. The final cell solution was counted and viability was assessed using Trypan Blue exclusion. The final cell count was calculated as the total number of viable cells per μL.

8 week old mice were used for this study. Animals were first anesthetized with isoflurane oxygen mixture, then the head of the animal was shaved and treated with betadine. Following mounting in a stereotaxic frame, a single midline incision was made along the scalp and skin retracted to expose bregma. A 10 μL Hamilton syringe was loaded with the cell solution. A small burr hole was drilled in the skull above the injection site in the right hemisphere (from bregma: anterior 1.0 mm and lateral 1.5 mm). The needle was slowly inserted into the brain (3.1 mm ventral to the pia mater in mice) and 1×10⁴ viable GL261 cells were injected at a speed of 0.5 μL/minute. Following injection, the needle remained in place for 1 minute. The injection needle was raised 0.1 mm and again 0.2 mm from the initial injection site and the injection was repeated with 1×10⁴ cells injected at each site for a total of 3×10⁴ viable cells across three sites. At the conclusion of the last injection, the needle remained in place for 3 minutes before being slowly withdrawn. Immediately following intracranial injection of GL261-Luc cells, hemostats were then inserted into the incision site and used to create a subcutaneous pouch immediately posterior to the scapula of the mouse by which the micro-osmotic pump (Alzet 1002) was inserted with the catheter tubing connected to the cannula (Alzet brain infusion kit 3) extending through the incision site. The cannula was slowly lowered into the brain though the same burr hole using a cannula holder (Alzet cannula holder 1) to sit 3 mm below the skull. Cannulas were fixed to the skull of mice using Loctite 454 and then the cannula guide was removed using bone shears. The incision was then closed using 4-0 absorbable sutures and mice were transferred to a heated recovery cage until fully sternal at which point mice were singly housed and returned to colony rooms. The pumps were loaded with either PBS, 70 μM of DOX (0.2 mg DOX/Kg mouse), nanotubes (NT) at 95 μM of ssDNA-amphiphiles, or NT-DOX at the same concentrations of DOX and amphiphiles. Mice were monitored twice daily for signs of advanced tumor progression. Mice were imaged weekly for 4 weeks after tumor implantation. The substrate D-luciferin (ThermoFisher) was administered via intraperitoneal injection (i.p.) at 150 μgr/gr body weight in 200 μL PBS. The mice were then placed onto the warmed stage inside the imaging chamber with continuous exposure to 1-1.5% isoflurane in 1 L/min oxygen. Bioluminescence images were acquired using the IVIS 1000 system (Xenogen) equipped with a highly sensitive cooled CCD camera, 10-15 minutes after D-luciferin administration. Images were analyzed by using the Living Image software (Xenogen). Regions of interest (ROI) were defined in the brain, which were held constant across all images. The photon counts within each ROI were quantified. For visualization purposes, the bioluminescent image and the corresponding white light surface image were fused into a transparent pseudo-color overlay. When a mouse reached a moribund state, the animal was deeply anesthetized via ketamine overdose (100 mg/kg, i.p.) and perfused with ice-cold PBS followed by 4% paraformaldehyde (PFA). The brain, heart, lung, kidneys, and spleen were removed from the animals and stored in PFA overnight at 4° C. and then transferred to 70% ethanol.

### Histopathological Analysis

Following perfusion and fixation with 10% neutral buffered formalin, tissues were processed into paraffin blocks using standard histology techniques, sectioned at a thickness of 4 m, stained with hematoxylin and eosin (H&E), and evaluated using light microscopy.

### μPET/CT Imaging of Orthotopic GL261 Tumor-Bearing Mice

Tumors were prepared in the same manner as for the bilateral intracranial injections of nanotubes. Fourteen days after GL261 implantation, nanotubes containing 5 mol % DOTA-labeled ssDNA-amphiphiles were mixed with ⁶⁴CuCl₂. The dried ⁶⁴CuCl₂ salt was dissolved in 100 mM sodium acetate in Milli-Q water (pH=6) at 2 μCi/μL. The nanotubes at 250 μM were diluted to 150 μM in 2×PBS and then mixed with the ⁶⁴Cu solution (1:1 v/v) giving final concentrations of 75 μM ssDNA-amphiphile and 1 μCi/L ⁶⁴Cu in 1×PBS. The mixture was incubated at 37° C. for 1 hour to allow for chelation of the radioisotope by the DOTA moieties as well as to pre-heat the solution prior to injection.

Mice were placed under a heat lamp prior to injections to dilate the veins in their tails. The tails were wiped with ethanol swabs to clean them prior to injection, and 200 μL of the ⁶⁴Cu-labeled nanotube solution was injected into the lateral tail veins of the mice. The final solution injected contained approximately 0.8 pmol of ⁶⁴Cu and 1.5 nmol of DOTA-labeled ssDNA-amphiphiles, approximately 1,850 times molar excess of DOTA to ⁶⁴Cu, which has been shown to entirely chelate all available copper. The radioactivity and time of measurement for each individual syringe was measured immediately before and after tail vein injections. 15 minutes prior to each imaging time point (1, 3, and 24 hours post injection), mice were anesthetized using 3% isofluorane in oxygen at 0.8 L/minute flow. μPET and CT scans were taken on a Siemens Inveon PET/CT scanner. After the 1 hour imaging time point, mice were placed under a heat lamp until they regained consciousness. After either the 3 hour or the 24 hour time point, mice were euthanized for ex vivo organ radioactivity measurements; if mice were not euthanized after the 3 hour time point, they were placed under a heat lamp until they regained consciousness.

Images from the μPET/CT scans were saved as DICOM files and cropped to separate each individual mouse (www.mevislab.de). Care was taken to maintain the coordinate system and the calibrated radiological values contained in the original DICOM files. From these separated images, volumetric 3D renderings of each mouse were created for the whole mouse body. ImageJ was used to create the maximum intensity projections of the head of each mouse and to plot the μPET intensity profiles as a function of distance across the head of the mouse, starting from the left hemisphere.

### Ex Vivo Biodistribution Analysis

At 3 or 24 hours post injections, mice were euthanized to collect organs for the biodistribution measurements. Organs were excised and weighed to determine their mass. The radioactivity of each organ (kilo counts per minute, kcpm) was recorded using a scintillator and converted to μCi using a calibration curve. The radiation values for each organ were then adjusted for the decay half-life of ⁶⁴Cu (12.7 hours). The total injected dose was calculated by measuring the decay-adjusted radiation in the syringe prior to the injection and subtracting the decay-adjusted radiation in the syringe after injection. Additionally, the decay-adjusted radiation in each mouse's tail at the time of euthanasia was subtracted due to the possibility of missing the vein during injection, thereby limiting the amount of ⁶⁴Cu systemically delivered. Organ radioactivity was scaled to the normalized injected dose and then scaled by the mass of the organ. Data were plotted as percent injected dose per gram of tissue (% ID/gr).

### Statistical Analysis

Statistical differences were determined using unpaired two-tailed Student's t-tests or one-way ANOVA with Tukey's honest significant difference post-hoc test. Survival Kaplan-Meier curves were constructed and compared using a two-sided log-rank test. Statistical analyses were performed using Excel (Microsoft) and the Real Statistics Excel Resource Pack.

### Example 2: Self-assembled ssDNA Nanotubes for Selective Targeting of Breast Cancer Cells and Delivery of Doxorubicin

This Example demonstrates that nanotubes formed from the self-assembly of ssDNA-amphiphiles can be used to selectively target breast cancer cells, including triple negative breast cancer (TNBC) cells, and deliver doxorubicin (DOX). For example, treatment with DOX intercalated in the nanotubes resulted in decreased cell viability of TNBC cells.

### Results and Discussion

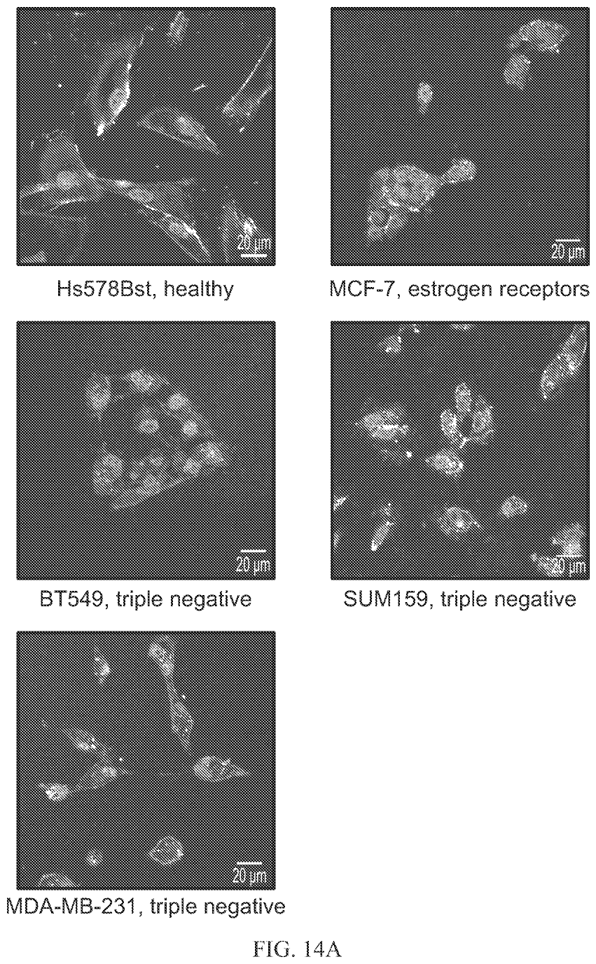

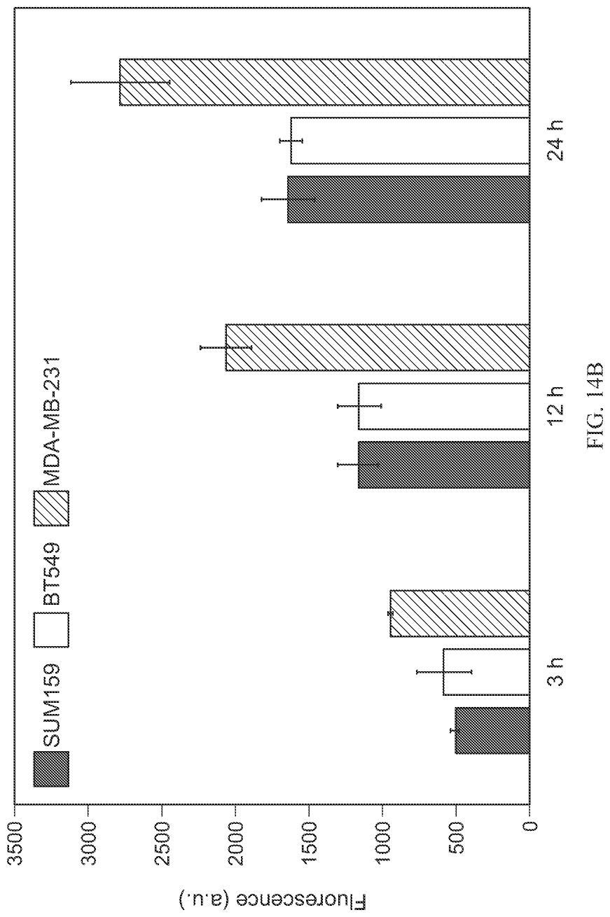

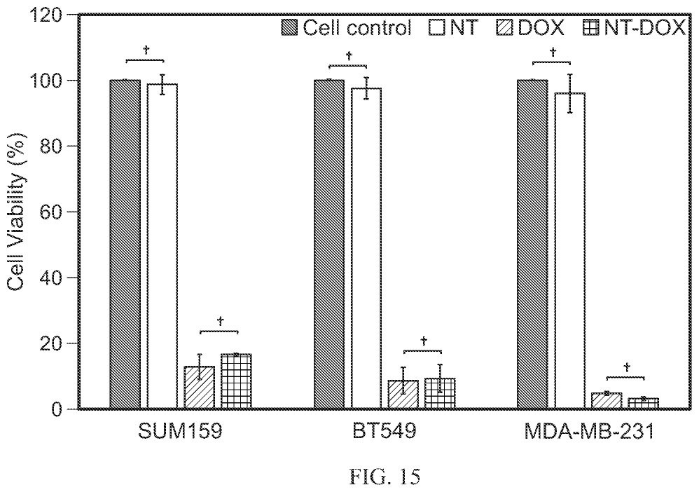

HEX-labeled nanotubes were incubated with Hs578Bst healthy human breast cells, MCF-7 human breast cancer cells that express estrogen receptors, and different TNBC cells (BT549, SUM159, MDA-MB-231) that do not have estrogen receptors, progesterone receptors and HER2, for 3 hours at 37° C. Confocal microscopy was used to determine qualitatively the extent of cell internalization (FIG. 14A). The nanotubes showed strong cell internalization into all breast cancer cells (MCF-7, BT549, SUM159 and MDA-MB-231) with minimal surface binding and no internalization into the healthy Hs578Bst cells. Flow cytometry was used to evaluate quantitatively the association of the nanotubes with the TNBC cells, and FIG. 14B shows that association of the nanotubes with the TNBC cells increased as a function of time. The effect of nanotubes (NT), DOX and DOX intercalated in the nanotubes (NT-DOX) on the viability of TNBC cells was also assessed (FIG. 15). The empty ssDNA nanotubes were shown to have no effect on cell viability, whereas when used to deliver DOX to BT549, SUM159 and MDA-MB-231 cells, they were as cytotoxic as free DOX.

Given the difficulty of treating TNBC, the development of our ssDNA nanotubes that can target TNBC cells could be highly impactful, as it may enable enhanced efficacy with reduced off-target effects for a variety of treatment options including gene therapy and traditional chemotherapeutics. This work shows that nanotubes self-assembled from ssDNA-amphiphiles can selectively target different breast cancer cells, including TNBC cells, and used for the delivery of a chemotherapeutic, such as DOX, thus making them a promising targeted drug delivery system.

### Methods

### Preparation of Nanotubes

The ssDNA-amphiphiles, HEX-labeled nanotubes and nanotubes intercalating DOX were prepared as described in Example 1.

### Cell Internalization of Nanotubes Examined Via Confocal Microscopy

50,000 cells were seeded on glass coverslips in a 12 well plate and allowed to attach overnight at 37° C. The next day, the media was replaced with 500 μL of fresh media and 5 nmol of 20% HEX-labeled nanotubes and let incubate for 3 hours. After 3 hours, the cells were fixed with 4% paraformaldehyde for 30 minutes. The membrane was then stained with AlexaFluor594 Wheat Germ Agglutinin (Thermo Fisher Scientific, Rockford, IL) at 10 μg/mL for 15 minutes. The nuclei were stained with Hoechst 33342 (Thermo Fisher Scientific, Rockford, IL) at 10 μg/mL for another 15 minutes. The coverslips were mounted onto glass slides using Prolong Gold and imaged with a Carl Zeiss LSM780 confocal microscope (Integrated Imaging Center, Institute for NanoBioTechnology).

### Cell Association of Nanotubes Examined Via Flow Cytometry

Cells were seeded at a density of 200,000 cells/well in a 12 well plate and allowed to adhere overnight at 37° C. The next day, 500 μL of fresh media and 5 nmol of 20% HEX-labeled nanotubes in PBS were added and let incubate for 3, 12 and 24 hours at 37° C. The cells were then washed with PBS, detached from the plate, washed again with PBS, and analyzed via flow cytometry (BC FACSCanto, Integrated Imaging Center, Institute for NanoBioTechnology). To ensure only live cells were being examined, 5-10 μL of a 10 μg/mL propidium iodide (PI) solution was added to each sample. Cells were gated by PI staining and by scattering (FCS-A and SSC-A). Association of labeled tubes was examined using the HEX fluorescence of the cells via excitation at 488 nm with a 585/42 filter.

### DOX Cytotoxicity

5,000 cells/well were seeded in white 96-well tissue culture treated plates in 100 μL media and allowed to adhere overnight at 37° C. The next day, the media was removed and replaced with 95 μL of new media and 5 μL of either nanotubes (NT), free DOX or DOX intercalated in the ssDNA nanotubes (NT-DOX). For the NT samples, 1.15 μM of ssDNA-amphiphiles were delivered to SUM159 and BT549 or 11.1 μM for MDA-MB-231, free DOX was delivered at 0.5 μg/mL for SUM159 and BT549 or 5 μg/ml for MDA-MB-231, and the NT-DOX formulations were delivered at the same DOX and amphiphile concentrations. The cells were incubated with the sample for 12 hours at 37° C., then washed with 100 μL PBS, and incubated with 100 μL of media for another 36 hours at 37° C. The plate was then removed from the incubator and allowed to equilibrate to room temperature for 30 minutes while CellTiter-Glo 2.0 was thawed in a room temperature water bath. 100 μL of CellTiter-Glo 2.0 was then added to each well. Per manufacturer instructions, the plate was shaken for 2 minutes and then allowed to rest for 10 minutes in darkness. The luminescence signal of each well was measured on the BioTek Synergy H1 microplate reader, and the luminescence of each treatment group was normalized to the luminescence of the untreated cells for that cell line.

### Example 3: Self-Assembled ssDNA Nanotubes for Targeting of Colon, Liver and Pancreatic Cancer Cells

This Example demonstrates that nanotubes formed from the self-assembly of ssDNA-amphiphiles can be used to target colon, liver and pancreatic cancer cells.

### Results and Discussion

FAM-labeled nanotubes were incubated with CT26 colon cancer cells, PANC-1 pancreatic cancer cells and HepG2 liver cancer cells for 3 hours at 37° C. Confocal microscopy was used to determine qualitatively the extent of cell internalization (FIG. 16). The nanotubes showed strong cell internalization into colon, pancreatic and liver cancer cells.

### Methods

### Preparation of Nanotubes

ssDNA-amphiphiles and FAM-labeled nanotubes were prepared as described in Example 1.

### Cell Internalization of Nanotubes Examined Via Confocal Microscopy

This experiment was performed as discussed in Example 2.

### Example 4: Anti-miRNA ssNA Nanotubes for Targeting Glioblastoma Cells and Sensitizing them to Doxorubicin

This Example demonstrates that nanotubes formed from the self-assembly of single-stranded nucleic acid (ssNA)-amphiphiles, where the ssNA sequence is that of an anti-miRNA, can be used to target glioblastoma (GBM) cells and sensitize them to doxorubicin (DOX). For example, treatment of the GBM cells with the anti-miRNA nanotubes resulted in decreased viability of the cancer cells after exposure to DOX.

### Results and Discussion

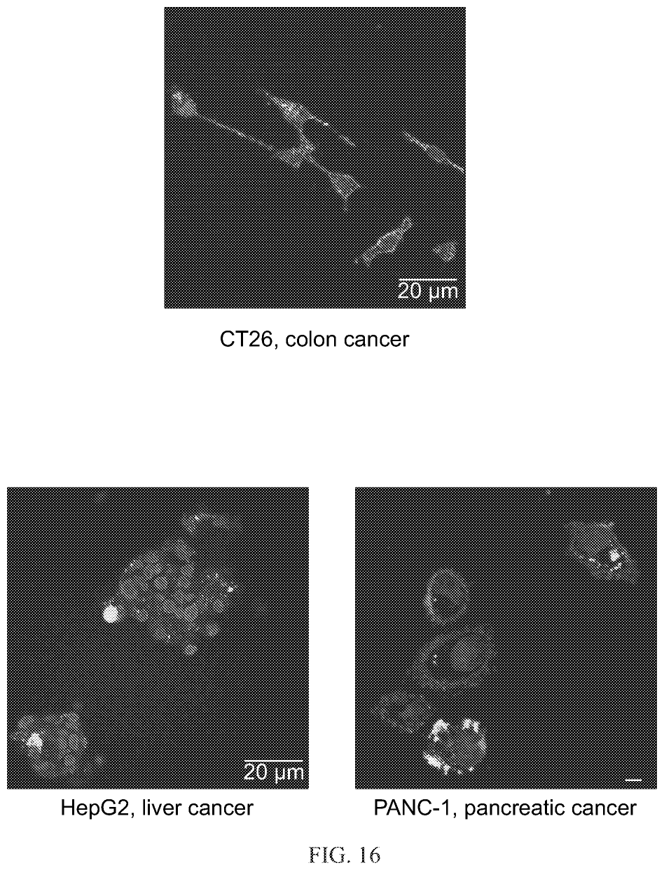

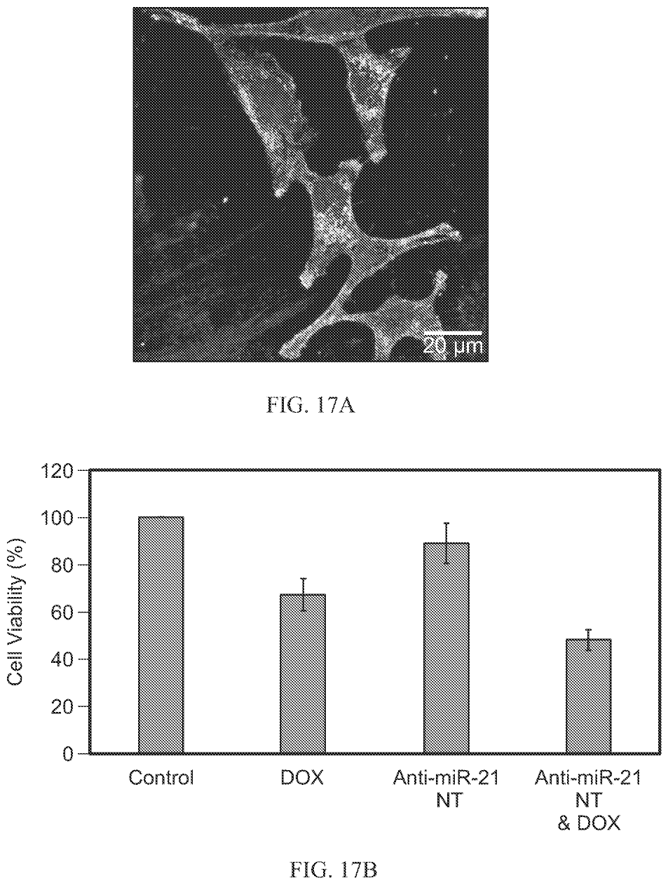

FAM-labeled anti-miR-21 nanotubes were incubated with A172 human GBM cells for 3 hours at 37° C. Confocal microscopy showed strong internalization of the anti-miR-21 nanotubes by the GBM cells (FIG. 17A). The ability of the anti-miR-21 nanotubes to chemosensitize the GBM cells was also evaluated. A172 cells were treated with the anti-miR-21 nanotubes for 24 hours. After incubation, media was replaced and cells were incubated with free DOX for 12 hours. After removing the media and washing the cells, fresh media was added and the cells were allowed to incubate for 36 hours. Results on FIG. 17b show that the anti-miR-21 nanotubes on their own decreased cell viability by 11% compared to the control, and when delivered to the cells before DOX, they decreased cell viability by 52% compared to the control. When comparing this treatment to DOX alone, the anti-miR-21 nanotubes were shown to increase cell death by 29% and thus were shown to effectively sensitize the GBM cells to DOX treatment and have a higher cytotoxicity than DOX alone.

### Methods

### Preparation of Anti-miR-21 Nanotubes

The following sequences were used for the synthesis of the ssNA-amphiphiles and FAM-labeled amphiphiles: 5′-AmMC6-TCAACATCAGTCTGATAAGCTA-3′ (SEQ ID NO 3) and 5′-AmMC6˜TCAA CATCAGTCTGATAAGCTA-3′-6˜FAM (SEQ ID NO:3). The amphiphiles, nanotubes and FAM-labeled nanotubes were prepared as described in Example 1.

### Cell Internalization of Nanotubes Examined Via Confocal Microscopy

20,000 A172 human GBM cells were plated per well in a 12-well plate onto 18 mm circular glass cover slips. They were then incubated for 24 hours at 37° C. and 5% C02. The media was then replaced with 500 μL fresh media and either PBS or 1 nmol FAM-labeled anti-miR-21 nanotubes were added. The cells were then placed back in the incubator for 3 hours. The cells were washed 3× with 1 mL PBS and then fixed with 4% paraformaIdehyde at room temperature for 30 minutes. The cells were washed 3× with 1 mL PBS and stained with 10 μg/mL Wheat Germ Agglutinin Alexafluor647 for 15 minutes at room temperature. Cells were washed 3× with PBS and stained with 10 μg/mL Hoechst for 15 minutes at room temperature. They were washed 2× with PBS and 1× with Milli-Q water before joining on glass slides using Diamond ProLong antifade mount and imaged on a Zeiss LSM700 confocal microscope (Integrated Imaging Center, Institute for NanoBioTechnology).

### Chemo-Sensitization Assay

5,000 A172 cells were plated per well (100 μL of 50,000 cells/mL) in a white 96-well cell culture treated plate. The cells were then incubated at 37° C. and 5% C02 for 24 hours. After that the media in all wells was replaced with fresh media. To this was added 10 μL of either Optimem serum free media or 10 μL of anti-miR-21 nanotubes in Optimem serum free media targeting a final concentration of 90 nM. The cells were then incubated for another 24 hours. After the incubation, the media was removed and replaced with either 95% media 5% PBS or 95% media 5% 4 μg/mL doxorubicin-HCl (DOX) in PBS (final targeted concentration 0.2 μg/mL DOX). The cells were then incubated for 12 hours before removing the media, washing once with PBS, and adding fresh media. The cells were then incubated for 36 hours. Relative proliferation was calculated using the CellTiter-Glo 2.0 assay utilizing the methods outlined by the manufacturer. The luminescence signal of each well was measured on the BioTek Synergy H1 microplate reader, and the luminescence of each treatment group was normalized to the luminescence of the untreated cells.

### Example 5: miRNA dsNA Nanotubes for Targeting Glioblastoma Cells

This Example demonstrates that nanotubes are formed from double-stranded nucleic acid (dsNA)-amphiphiles, where the dsNA sequence is that of a miRNA duplex or siRNA duplex. For example, miRNA nanotubes were prepared and used to target glioblastoma (GBM) cells.

### Results and Discussion

miRNA or siRNA nanotubes were made by the self-assembly of dsNA-amphiphiles, where the dsNA sequence is that of a miRNA duplex or siRNA duplex. The dsNA-amphiphiles were prepared by conjugating the dsNA to the hydrophobic tail-spacer molecule, or by preparing the ssNA-amphiphile and then hybridizing its complementary sequence to the amphiphile.

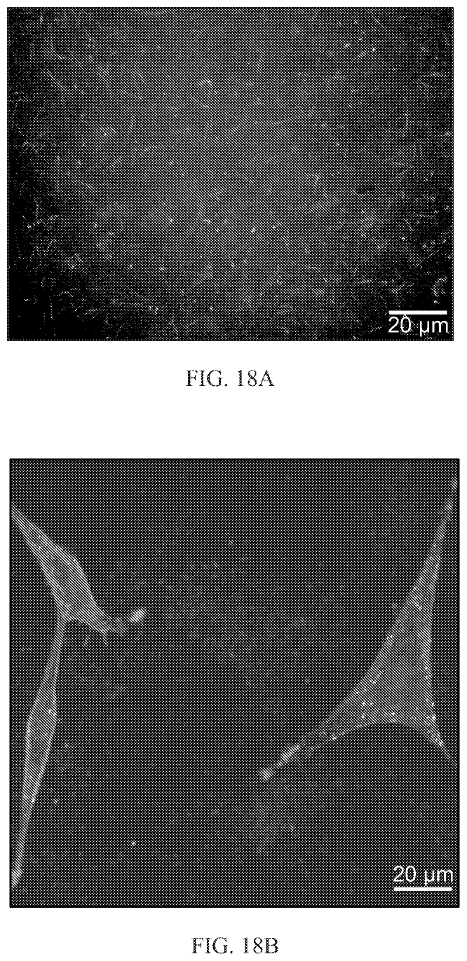

In this example, nanotubes were prepared from the self-assembly of ssNA-amphiphiles. The ssNA sequence is the guide miRNA-21 (miR-21). Duplex miR-21 nanotubes were prepared by hybridizing its complementary sequence (anti-miR-21) to the ssNA-amphiphiles within the pre-formed nanotubes. The complementary sequence had a FAM fluorophore, thus allowing for the visualization of the duplex miR-21 nanotubes via fluorescence microscopy (FIG. 18A). The nanotubes composed of the miR-21 duplexes were used to target GBM cells. FAM-labeled miR-21 nanotubes were incubated with A172 human GBM cells for 3 hours at 37° C. Confocal microscopy showed strong internalization of the miR-21 nanotubes by the GBM cells (FIG. 18B).

### Methods

Preparation of miR-21 dsNA Nanotubes

ssNA amphiphiles were synthesized as described in Example 1 by using the guide miR-21 sequence with an extra G5 at the conjugation end, 5′-TAGCTTATCAGACTGATG TTGAGGGGG-AmMO-3′ (SEQ ID NO:4). The nanotubes were prepared as described in Example 1. The complementary sequence to miR-21 was ordered with a FAM fluorophore (anti-miR-21: 5′-TCAACATCAGTCTGATAAGCTA-3′-6-FAM; SEQ ID NO:3).

Nanotubes at 1 nmol of ssNA-amphiphdles were mixed with 1 nmol of the free complementary sequence that was fluorescently labeled. The sample was left at room temperature overnight to hybridize, and the next day 1 μM of the sample was imaged with a fluorescent microscope.

### Cell Internalization of Nanotubes Examined Via Confocal Microscopy

This experiment was performed as discussed in Example 4.

### Example 6: Hybrid Peptide-Nucleic Acid Nanotubes for Targeting Cancer Cells and Delivering Nucleic Acids

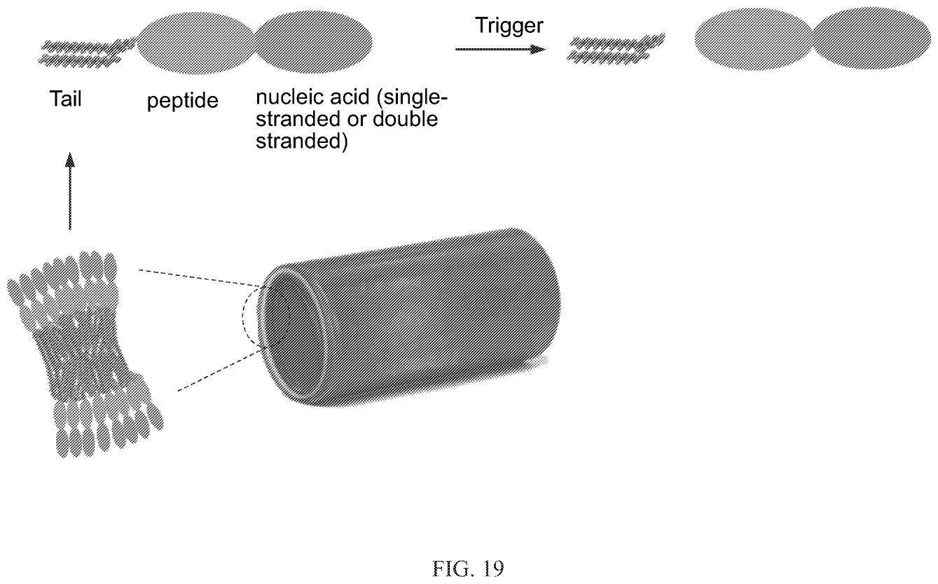

This Example describes (peptide-NA)-amphiphiles that self-assemble into nanotubes. The amphiphiles can be static or dynamic that can release the peptide and NA (single-stranded or double-stranded) under a specific trigger, such as NIR light or pH (FIG. 19). The peptide is included in the design of the amphiphile to promote escape of the NA from endosomes and lysosomes after cell internalization. Such nanotubes can be used to deliver ssDNA, dsDNA, siRNA, and miRNA (mimics or antagonists) having a therapeutic tumor suppressive function to cancer cells.

For the synthesis of the static (peptide-NA)-amphiphiles, the peptide is conjugated to the NA as described elsewhere (see, e.g., Wickramathilaka and Tao, J. Biol. Eng., 13, 63 (2019)), and the (peptide-NA)-amphiphile is synthesized as described elsewhere (see, e.g., Mardilovich et al., Langmuir, 22:3259-3264 (2006); and Pearce and Kokkoli, Soft Matter, 11:109-117 (2015)). For the synthesis of the dynamic (peptide-NA)-amphiphiles, spacers that are sensitive to NIR light (see, e.g., Yang et al., Colloids Surf B, 128:427-438 (2015)) or pH (see, e.g., Gillies et al., Bioconjug. Chem. 15:1254-1263 (2004)) are used between the tail-spacer and (peptide-NA), or between each building block: the tail-spacer and (peptide-NA) and between the peptide and NA. The critical micelle concentration of the resulting amphiphiles and their charge are evaluated. To evaluate the release profiles of the NAs from the nanotubes under NIR light or pH (5-6), the NAs are labeled with a fluorophore. When the fluorescently labeled NAs are conjugated to the nanotubes the solution is fluorescent; however, after exposure to NIR light or pH solution the NAs released from the nanotubes and the sample has a decreased fluorescence. The exposure time needed to release all NAs from the nanotubes is determined for different NA concentrations. Morphology of the assembled structures before and after exposure to a trigger is assessed via cryo-TEM. The stability of the nanotubes is evaluated via gel electrophoresis after exposure to triggers, serum, and different concentrations of DNA and RNA nucleases.

Specific binding to different cancer cells, cell internalization, and trafficking of nanotubes are evaluated. Appropriate healthy cells are used as controls. Binding and internalization are evaluated via flow cytometry and confocal microscopy. Trafficking of nanotubes is evaluated by blocking endocytosis with different agents, and visualizing colocalization of the nanotubes with different organelles via confocal microscopy. The effect of NIR light is also evaluated in the trafficking of the nanotubes. Cytotoxicity and cell apoptosis is evaluated in the presence and absence of NIR light using metabolic assays, live-dead assays and caspase assays.

### Example 7: Formation of NA Nanotubes from NA Globular Micelles

As discussed in Example 1, NA-amphiphiles self-assemble into small micelles (spherical/ellipsoidal) and nanotubes. This Example describes methods that can increase the formation of microns-long nanotubes from a sample that has small spherical/ellipsoidal micelles and a few short nanotubes. This Example also describes methods for shortening long nanotubes using probe sonication.

### Results and Discussion

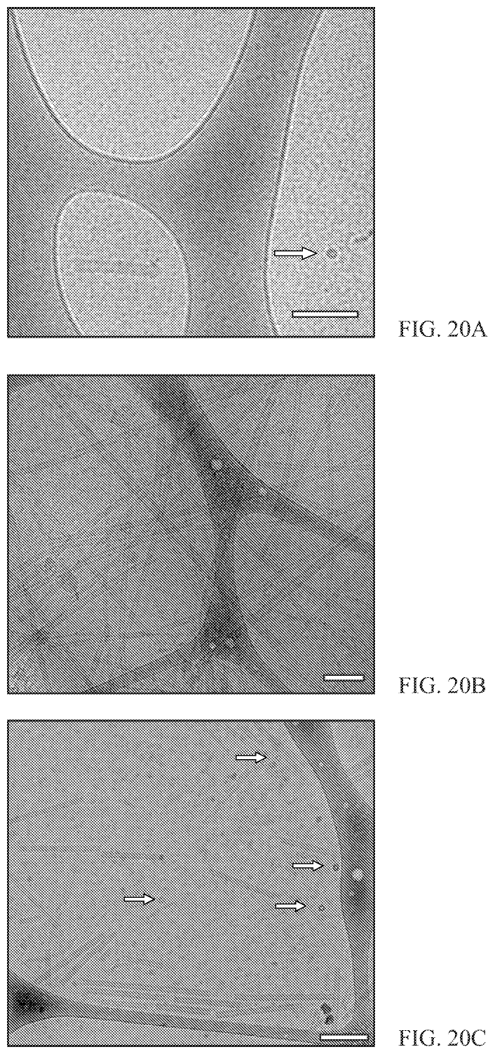

ssDNA-amphiphiles were synthesized as described in Example 1. Cryo-TEM imaging verified the presence of many small globular micelles and short nanotubes (FIG. 20A). The excess tail method was used to form long nanotubes from micelles. Cryo-TEM images showed that with this method shifted the amphiphile distribution towards microns-long nanotubes (FIG. 20B). The long nanotubes were shortened to the desired length using probe sonication. Cryo-TEM analysis of the shortened tubes (FIG. 20C) found them to measure 319±126 nm in length and 30-50 nm in diameter.

### Methods

### Preparation of Long Nanotubes Via the Excess Tail Method

50-250 μM ssDNA-amphiphiles that self-assemble into small micelles were first neutralized by combining them in a 1:0.1:3 volume ratio of aqueous amphiphile:3M sodium acetate pH 5.2: ethanol, vortexing after each addition. The amphiphile solution was then cooled to −80° C. for at least 1 hour to ensure precipitation and collected via centrifugation at 16,100 RCF and 4° C. for 45 minutes. The supernatant was then removed, and the pellet was washed twice with 75% ethanol, 25% Milli-Q water (centrifuging for 10 minutes between each wash), and dried in a vacuum oven at 40° C. The dried, neutralized amphiphiles were then combined with 10-20× molar excess dialkyl (C₁₆)₂ tail with attached C₁₂ spacer in 65° C. DMSO, stirred for at least 2 hours at 65° C., and added drop-wise to Milli-Q water while mixing rapidly with a stir bar. The residual DMSO was then removed via dialysis using a 1,000 MWCO Medi Tube-O-DIALYZER (G-Biosciences, St Louis, MO) or a second ethanol/acetate purification, matching the first step except resuspending in MilliQ water instead of drying in a vacuum oven.

### Shortening of Nanotubes Via Probe Sonication

Solutions of long nanotubes were diluted to a volume of at least 500 μL in a 15 mL conical tube or 2 mL in a 50 mL conical tube. The tube was then clamped in place in an ice bath to prevent large temperature fluctuations. A Q125 or Q500 probe sonicator (Qsonica, Newtown, CT) with a ⅛ inch diameter probe was used for sonication. The tip of the probe was submerged within the solution such that the end was near the bottom of the tube but was not touching either the walls or bottom of the tube. The sample was then sonicated on the 40% amplitude or 20% amplitude setting for 1 minute total of sonication in pulses of 10 seconds on and 10 seconds off. 40% amplitude was shown to produce slightly shorter nanotubes than 20% amplitude and could therefore be utilized when shorter nanotubes are desired.

### Cryogenic Transmission Electron Microscopy (Cryo-TEM)

The nanostructures formed by the NA-amphiphiles were examined via cryo-TEM as described in Example 1.

### Example 8: Hydrophobic Molecules Encapsulated in the Wall of the NA Nanotubes Can Kill Senescent and Proliferating Cancer Cells and Repolarize Macrophages

This Example demonstrates methods for encapsulating hydrophobic molecules used to treat cancer (e.g., chemotherapeutics and senolytics) in the hydrophobic wall of the ssDNA nanotubes. The Example also demonstrates that such nanotubes encapsulating hydrophobic molecules used to treat cancer (e.g., chemotherapeutics and senolytics) can be used to kill senescent cancer cells and/or proliferating cancer cells, and can be used to re-polarize tumor-associated macrophages.

### Results and Discussion

Senolytics Encapsulated in ssDNA Nanotubes Kill Senescent Cancer Cells and Sensitize them to Chemotherapy

ABT-263, a hydrophobic senolytic, can sensitize senescent cells to doxorubicin (DOX) such that dual delivery of ABT-263 and DOX can lead to an additive effect, and could prolong the effectiveness of DOX treatment. The dual delivery of both these drugs using a single targeted system could therefore mean a much more efficacious treatment, with a decreased risk of recurrence.

Successful encapsulation of ABT-263 in the ssDNA nanotubes was achieved with an average encapsulation efficiency of 89%. An examination of the release kinetics showed burst release of ˜30% over the first 4 hours, followed by a much slower release after that. ABT-263 was released from the nanotubes in a period of 30 days.

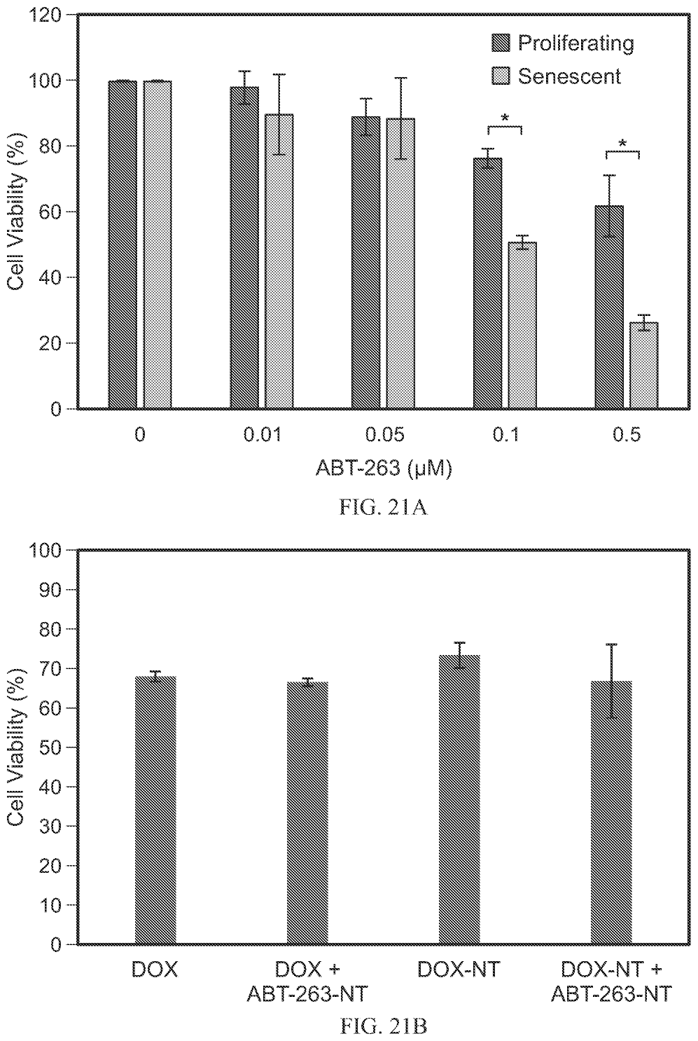

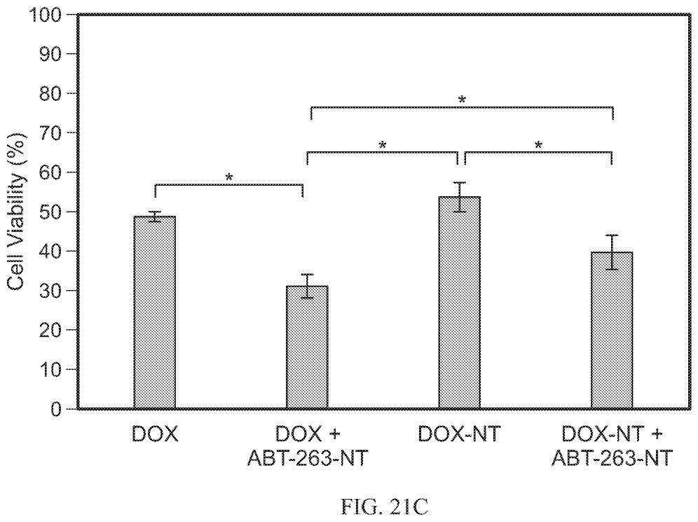

The nanotubes encapsulating ABT-263 were delivered to proliferating and senescent triple negative breast cancer (TNBC) cells. A range of ABT-263 concentrations encapsulated in the nanotubes was delivered to either proliferating or senescent MDA-MB-231 TNBC cells for 48 hours (FIG. 21A). ABT-263 encapsulated in the nanotubes was more cytotoxic to senescent cells than proliferating MDA-MB-231 cancer cells. A combination of either free DOX or DOX intercalated in the nanotubes plus ABT-263 encapsulated in the nanotubes were delivered to proliferating (FIG. 21B) and senescent (FIG. 21C) MDA-MB-231 cancer cells in separate nanotubes at 0.5 μg/mL DOX and 0.1 M ABT-263. For both free DOX and DOX-nanotube treatments, the addition of ABT-263-nanotubes significantly decreased senescent cell viability without significantly affecting proliferating cell viability, showcasing ABT-263's ability to target senescent cells (FIG. 21C).

Hydrophobic Drugs Encapsulated in ssDNA Nanotubes Kill GBM Cells

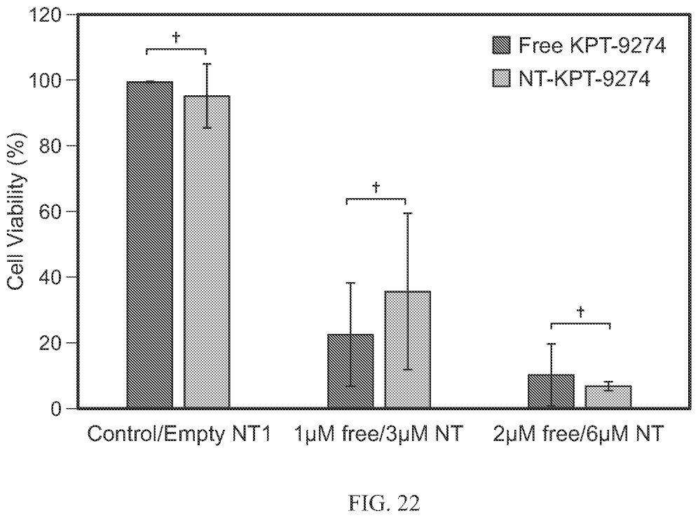

KPT-9274 was delivered to human U87 GBM cells, either free or encapsulated in the ssDNA nanotubes. Results show that delivery of KPT-9274 through the nanotubes was as effective as the free drug in killing GBM cancer cells (FIG. 22).

ssDNA Nanotubes Repolarize TAMs

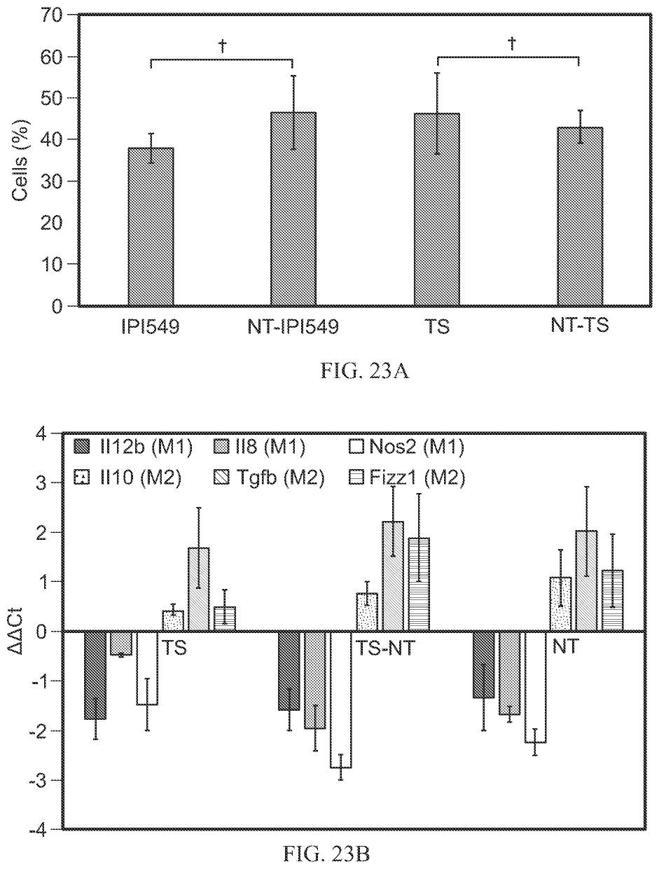

ssDNA nanotubes were used to repolarize TAMs from an M2-phenotype (pro-tumor phenotype found in tumors) to an M1-phenotype (anti-tumor phenotype), after encapsulating in the nanotubes either IPI-549 or thiostrepton (TS). Freshly isolated primary CD14+human monocytes were first differentiated into MO macrophages and then into M2 macrophages. Finally, different treatments (IPI-549 or TS, free or encapsulated in the nanotubes) were delivered to the M2-phenotype macrophages. At the end of treatment, cells were analyzed for surface markers CD163 and CD206 (present on M2-like macrophages), and CD80 and CD86 (present on M1-like macrophages) using flow cytometry. Results showed that there was no significant statistical difference between the number of M2-macrophages that were repolarized into an M1-phenotype after treatment with molecules (IPI-549 or TS) delivered free or encapsulated in the nanotubes (FIG. 23A).

The ability of different treatments to change the expression of phenotype specific genes (e.g., 1110, Tgfb and Fizzi for M2-macrophages, and 112b, 118 and Nos2 for M1-macrophages) was examined at the mRNA level via RT-qPCR. M2-like macrophages were treated with free TS, TS encapsulated in the nanotubes (TS-NT) and empty ssDNA nanotubes (NT). FIG. 23B shows that all three treatments (including the empty ssDNA nanotubes, NT) were successful at downregulating genes associated with M2-like macrophages (as shown by positive ΔΔCt) and upregulating genes of M1-like macrophages (as shown by negative ΔΔCt) compared to untreated M2-macrophages.

### Methods

### Encapsulation of Hydrophobic Molecules

50-250 μM ssDNA-amphiphiles were first neutralized by combining them in a 1:0.1:3 volume ratio of aqueous amphiphile:3M sodium acetate pH 5.2: ethanol, vortexing after each addition. The amphiphile solution was then cooled to −80° C. for at least 1 hour to ensure precipitation and collected via centrifugation at 16,100 RCF and 4° C. for 45 minutes. The supernatant was removed and the pellet was washed twice with 75% ethanol, 25% Milli-Q water (centrifuging for 10 minutes between each wash), and dried in a vacuum oven at 40° C. The dried, neutralized amphiphiles were then combined with 5-20× molar excess dialkyl (C₁₆)₂ tail with attached C₁₂ spacer in 65° C. DMSO and varying concentrations of hydrophobic drug (e.g., ABT-263, paclitaxel, KPT-9274, thiostrepton, IPI-549), stirred for at least 2 hours at 50-65° C.

In a first method, the solution with the hydrophobic molecules was added drop-wise to Milli-Q water while mixing rapidly with a stir bar. The residual DMSO was then removed via dialysis using a 1,000 MWCO Medi Tube-O-DIALYZER (G-Biosciences, St Louis, MO).

In a second method, the solution with the hydrophobic molecules was dried overnight via airflow to form a thin film and remove all DMSO. The sample was then rehydrated with Milli-Q water at 40° C. on a rotary evaporator device (no vacuum).

### Evaluating Effect of ABT-263 and DOX on Proliferating and Senescent Cancer Cells

5,000 MDA-MB-231 cells/well were seeded in white 96-well tissue culture treated plates in 100 μL media and allowed to adhere overnight at 37° C. The next day, the media were removed and replaced with either 100 μL of new media or 100 μL of media containing 0.05 μg/mL DOX for 3 days to induce senescence. The cells were then washed with 100 μL 1× PBS and incubated with 100 μL of new media containing the desired concentration of ABT-263 encapsulated in the nanotubes, for another 48 hours. For the combination treatment, DOX at the desired concentration, free or in the nanotubes, was also added at this point. Cell viability was assessed using the CellTiter-Glo 2.0 assay (Promega, Madison, WI) according to the manufacturer's instructions.

### Proliferation Study

Cells were plated at 5,000 cells per well in white 96-well plates and incubated for −24 hours. The next day the media were replaced, and the different treatments were spiked in at various concentrations. The cells were then incubated for the indicated time before analyzing by CellTiter Glo 2.0 assay according to the manufacturer's instructions.

### Primary Macrophage Polarization Study

Freshly isolated primary CD14+human monocytes were plated at a density of 500,000 cells/well in the presence of 50 ng/mL of M-CSF (macrophage colony stimulating factor) to differentiate them to MO-macrophages. The media were renewed with the same composition (DMEM+10% FBS+1% Penicillin-Streptomycin+50 ng/mL M-CSF) every 2-3 days until complete differentiation. The macrophages were treated with 20 ng/mL of IL-4 for 3 days differentiate them to an M2-phenotype. 100 nM IPI-549 or 1 μM thiostrepton (TS) were added (free or encapsulated in ssDNA nanotubes) in the presence of IL-4 to repolarize the macrophages to an M1-phenotype.

### Evaluation of Surface Markers Via Flow Cytometry

Cells were detached using 1× Tryp1E Express Enzyme (ThermoFisher Scientific) and washed with PBS. The cell suspensions were treated with Human TruStain FcX (Biolegend) FcR blocking reagent for 15 minutes at room temperature. The cells were then stained with FITC anti-human CD206 (Biolegend), PE anti-human CD163 (Biolegend), PE-Cy5 anti-human CD80 (Biolegend), and APC anti-human CD86 (Biolegend). Fluorescence Minus One (FMO) controls were prepared by splitting the cell suspensions prior to staining, and staining with the respective antibodies. Single color compensation controls were prepared by adding respective antibodies to one drop of UltraComp eBeads Plus Compensation beads (ThermoFisher Scientific). Cells were analysed on the BD FACS Canto and data analysis was conducted on FlowJo.

### Evaluation of Macrophage Gene Expression Via RT-qPCR

Cells were detached using 1× Tryp1E Express Enzyme (ThermoFisher Scientific) and washed with PBS. TRIzol (ThermoFisher Scientific) was added to the cells followed by absolute ethanol. mRNA was collected in Zymo-Spin IC columns (Zymo Research) by centrifuging the cell solution in the spin columns. The mRNA was washed and resuspend in RNAse and DNAse free water. After evaluating the mRNA concentration, the cDNA reaction mixture was prepared by adding the mRNA to nuclease-free water, iScript Reverse Transcriptase (Bio-Rad) and 5× iScript Reaction Mix. The cDNA reaction was performed using TurboCycler Lite Thermal Cycler (Blue-Ray Biotech). The cDNA was then aliquoted and added to a mixture of primers for the genes 1112b, 118, Nos2, 1110, Tgfb1 and Fizz1 respectively along with iTaq™ Universal SYBR® Green Supermix (Bio-Rad). The housekeeping genes used for the primary human macrophages were Gapdh and Rp137a. The polymerase chain reaction (PCR) was performed with CFX384 Touch Real-Time PCR Detection System (Bio-Rad). Data was analysed using the ΔΔCt method.

### Example 9: Nanotubes Formed Via Layer-by-Layer and Composed of Anti-microRNA or

Anti-microRNA and MicroRNA, are Used to Change the Expression of Genes of Interest, Minimize Cancer Cell Migration, Kill Cancer Cells, and Repolarize Macrophages This Example uses a layer-by-layer (LBL) approach to form nanotubes composed of anti-microRNA or anti-microRNA and microRNA. One of the layers is polyethylenimine (PEI), used to allow escape of the nanotubes from vesicles (endosomes and lysosomes) after cell internalization. The LBL nanotubes can downregulate or upregulate genes of interest to minimize cancer cell migration, decrease cancer cell proliferation, and/or repolarize TAMs.

### Results and Discussion

### Synthesis and Characterization of LBL Nanotubes

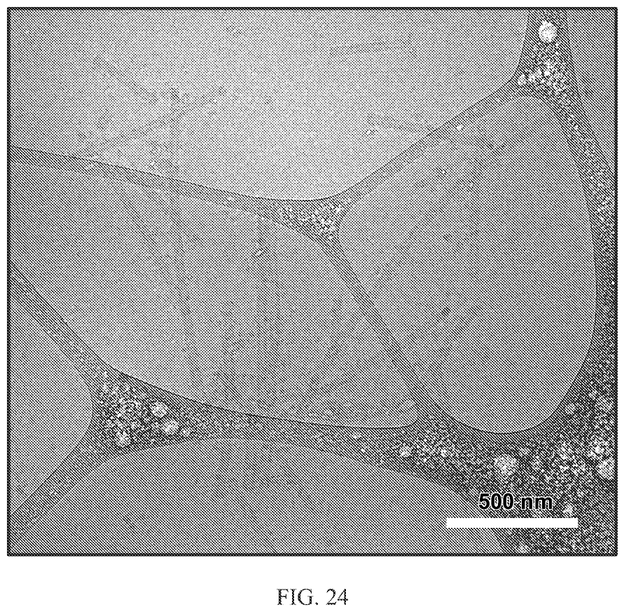

Anti-miR-21 amphiphiles were synthesized with anti-miR including locked nucleic acids (LNAs). Mixed ssDNA/LNA nucleotides were used as they can successfully sequester the targeted miR in a heteroduplex with high affinity. A LBL approach was used to generate anti-miR-21 nanotubes covered with a layer of PEI and then a layer of fucoidan or ssDNA (the 1OntGS ssDNA sequence was used from Example 1). Fucoidan can bind to a variety of receptors such as, different scavenger receptors, toll-like receptors, C-type lectins, selectins, integrins, vascular endothelial growth factors and their receptors, chemokines, elastin peptide receptor, extracellular matrix proteins and transforming growth factor-β (TGF-β) (see, e.g., Lin et al., Marine Drugs, 18:376 (2020)). PEI can enhance endosomal escape, thus allowing the anti-miR-21 to interact with its target miR-21 in the cytoplasm, rather than getting degraded in the lysosomes. Fucoidan or ssDNA that bind to scavenger receptors are present on the outer layer of the nanotubes to give specificity for the cancer cells. To verify successful coverage of each layer, the zeta potential of the nanotubes was measure at each step, after addition of a layer (Table 2). Cryo-TEM imaging was used to verify the presence of nanotubes at the end of the LBL process (FIG. 24).

TABLE 2
Zeta potential of LBL nanotubes after addition of each layer

| | | Zeta potential |
|---|---|---|
| Sample | Mass ratio | (mV) |
| Anti-miR-21 NT (NT) | 1 (NT) | −51.5 ± 2.9 |
| NT + PEI | 1 (NT):0.75 (PEI) | 30.7 ± 2.0 |
| NT + PEI + Fucoidan (NT-F) | 1 (NT):0.75 (PEI):5 | −33.7 ± 3.2 |
| | (Fucoidan) | |
| NT + PEI + 10ntG₅ ssDNA | 1 (NT):0.75 (PEI):3 | −36.6 ± 0.9 |
| (NT-10) | (ssDNA) | |

### LBL Nanotubes can Effectively Change the Expression of Target Genes in Different Cancer Cells, can Minimize Cancer Cell Migration, Kill Cancer Cells and Repolarize Macrophages.

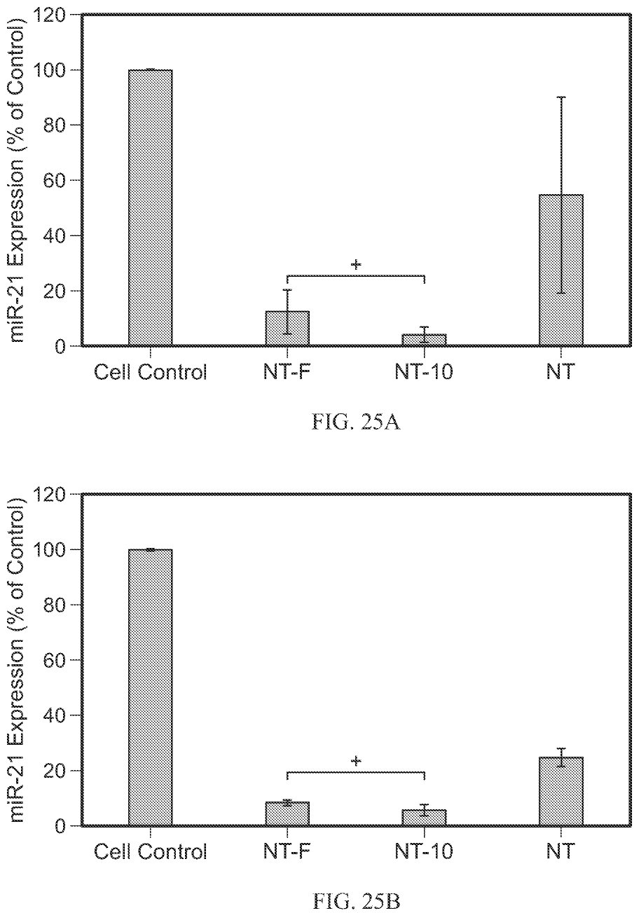

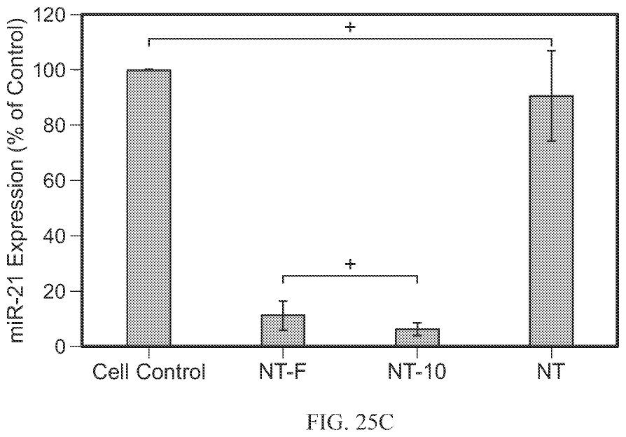

Anti-miR-21 nanotubes (NT) and LBL anti-miR-21 nanotubes with either an outer layer of fucoidan (NT-F) or the 10ntGS ssDNA sequence (NT-10) were delivered to different cancer cells and their ability to downregulate miR-21 at the mRNA level was examined via RT-qPCR. Results showed that both NT-F and NT-10 nanotubes, successfully downregulated miR-21 in U87 GBM cells (FIG. 25A), MDA-MD-231 TNBC cells (FIG. 25B) and Panc 10.05 pancreatic cancer cells (FIG. 25C). However, the ability of the anti-miR-21 nanotubes (NT) to downregulate miR-21 varied depending on the cell type, suggesting that the presence of the PEI layer can increase transfection.

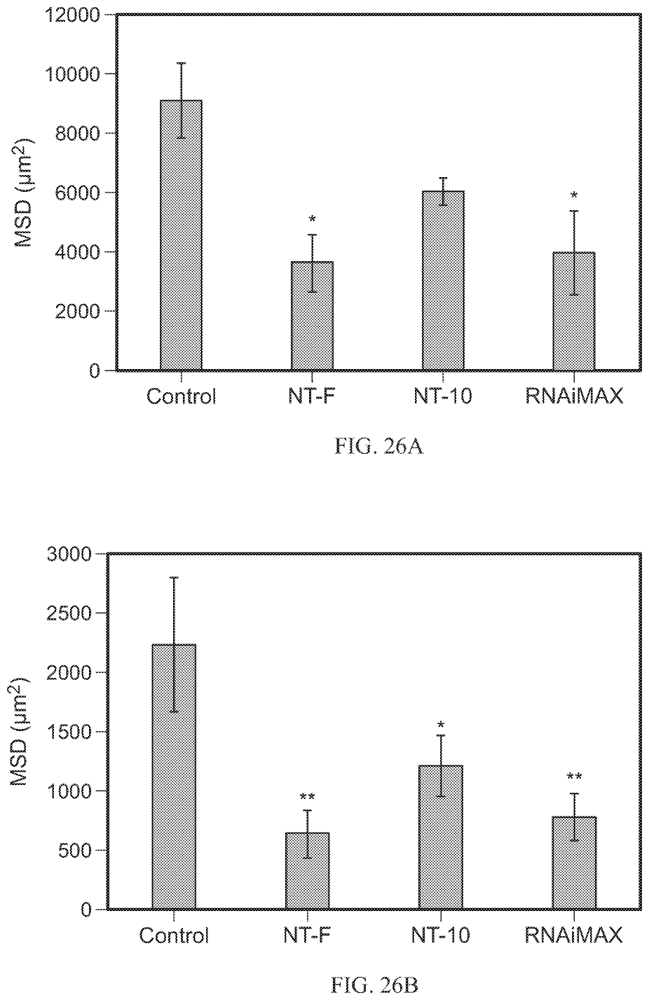

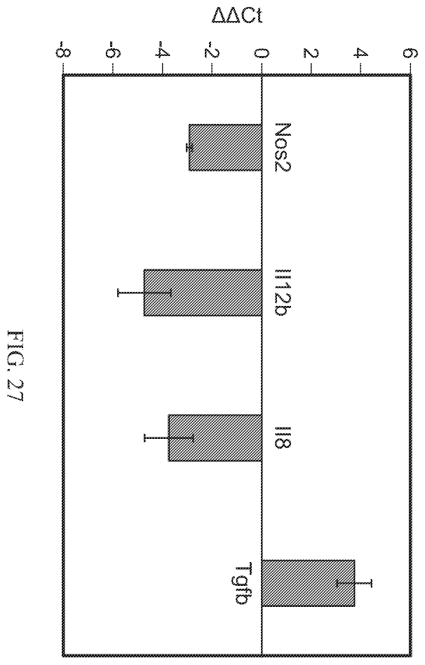

NT-F and NT-10 LBL anti-miR-21 nanotubes were effective at minimizing migration of different cancer cells (FIG. 26). NT-F were shown to be the most effective at decreasing the mean squared displacement of U87 GBM cells (FIG. 26A) and MDA-MB-231 TNBC cells (FIG. 26A), with no significant statistical difference between anti-miR-21 delivered via the NT-F nanotubes and the transfection agent RNAiMAX.

NT-F nanotubes were also evaluated for their ability to repolarize M2-like macrophages to MI-like macrophages as done in Example 8. RT-qPCR experiments showed that the NT-F nanotubes were successful at downregulating genes associated with M2-like macrophages (as shown by positive ΔΔCt) and upregulating genes of M1-like macrophages (as shown by negative ΔΔCt) compared to untreated M2-macrophages (FIG. 27).

LBL nanotubes were designed that carried both an anti-microRNA and a microRNA. The design featured the anti-miR-21 nanotubes, followed by layers of PEI, miR-603, PEI and fucoidan. The nanotubes effectively changed the expression of both target genes at the mRNA level in U87 GBM cells, as they downregulated the expression of miR-21 (ΔΔCt=3.29, 0.1 fold miR-21 expression) and upregulated the expression of miR-603 (ΔΔCt=−10.70, 1,663.5 fold miR-603 expression). These nanotubes were also cytotoxic to U87 GBM cells, and toxicity increased as a function of concentration (Table 3).

TABLE 3
Cell cytotoxicity of anti-miR-21 and
miR-603 present in LBL nanotubes

| Anti-miR-21 | MIR-603 | Cell proliferation |
|---|---|---|
| (nM) | (nM) | (% of control) |
| 0 | 0 | 100 (control) |
| 26 | 45 | 98 |
| 53 | 90 | 92 |
| 106 | 180 | 88 |
| 159 | 270 | 83 |
| 318 | 540 | 64 |

### Methods

### Layer-by-Layer (LBL) Assembly

Anti-miR-21 amphiphiles were synthesized as described in Example 1. The anti-miR-21 sequence was a mixture of ssDNA and LNA (5′AmMC₆/+T+C+AACATCAGTCTG ATAA+G+C+TA-3′ (SEQ ID NO:3), seed region underlined, LNA shown with+). The excess tail method described in Example 7 was then used to generate microns-long nanotubes from the amphiphiles, which were then shortened via probe sonication as described in Example 7. 3-20 nmol of these samples were then spiked with HEPES buffer to a final concentration of 25 mM HEPES pH 7.4. 25 kDa branched PEI (Sigma Aldrich, St. Louis, MO) was solubilized in 25 mM HEPES, spiked into the stirring solution of amphiphiles and allowed to stir for 20-30 minutes. Depending on the desired subsequent layer this solution was then spiked with ssDNA or fucoidan and stirred for another 20-30 minutes. The optimized mass ratio for spiking was determined to be 1:0.75:3 antimiR-21 nanotube core:PEI:ssDNA (l0ntG5 sequence used in Example 1) or 1:0.75:5 antimiR-21 nanotube core:PEI:fucoidan. This represented the final product for NT-10 (outer layer of 10ntG5 ssDNA sequence) and NT-F (outer layer of fucoidan). The five-layer construct that contained miR-603, additional layers of 25 kDa branched PEI and fucoidan were added in a similar manner, such that the final mass ratio was 1:0.75:3:1:5 antimiR-21 nanotube core:PEI:miR-603:PEI:fucoidan. The zeta potential of the nanotubes (evaluated via electrophoretic light scattering) was measured using a Zetasizer Nano ZS (Malvern Panalytical, Westborough, MA).

### Cryogenic Transmission Electron Microscopy (Cryo-TEM)

5 μL of amphiphile solutions (100-200 μM in 25 mM HEPES pH 7.4) were deposited onto lacey formvar/carbon copper grids that had been treated with glow discharge and vitrified in liquid ethane by Vitrobot (Vitrobot parameters: 3-5 seconds blot time, 0 offset, 3 seconds wait time, 0-3 seconds relax time, 95-100% humidity). After vitrification, the grids were kept under liquid nitrogen and were transferred to a F200C Talos TEM operated at an acceleration voltage of 200 kV (Integrated Imaging Center at the Johns Hopkins University Institute for NanoBioTechnology). Images were captured using a Ceta camera.

### Evaluation of Cancer Cell Gene Expression Via RT-qPCR

Cells were plated at 200,000 cells per well in a 6-well plate, incubated overnight, then treated for 48 hours. Cells were trypsinized and washed twice with PBS. TRIzol (ThermoFisher Scientific, Rockford, IL) was added to the cells followed by absolute ethanol. mRNA was collected using a Direct-zol RNA MicroPrep mrNA isolation kit (Zymo Research, Irvine, CA). The isolated mRNA in RNAse/DNAse free water was analyzed by UV-VIS spectrometry using a Synergy H1 plate reader (BioTek, Winooski, VT). The mRNA was diluted using Milli-Q water to 5 ng/L, and cDNA synthesis was completed using a miRCURY LNA RT kit (Qiagen, Germantown, MD). For cDNA synthesis, the mRNA was combined with 5× miRCURY RT SYBR green reaction buffer, 10× miRCURY RT Enzyme mix, UniSP6 RNA Spike-in Template, and Milli-Q water, and then thermo-cycled according to manufacturer's instructions. cDNA was combined with miRCURY LNA miRNA PCR assays (UniSP6, miR21, or miR603), 2× miRCURY SYBR green master mix, and Milli-Q water, and polymerase chain reaction (PCR) was performed with CFX384 Touch Real-Time PCR Detection System (Bio-Rad, Hercules, CA). miR21 or miR603 expression was normalized to the UniSP6 spike in expression and compared to the untreated control.

### Migration Study

MDA-MB-231 TNBC or U87 GBM cells (50,000 cells/mL) were embedded in 2 mg/mL collagen I gel as described elsewhere (Fraley et al., Sci. Rep., 5:14580 (2015)). The cells were treated for 72 hours with only media (control), 270 nM anti-miR-21 LBL nanotubes (NT-F and NT-10), or free anti-miR-21 complexed with RNAiMAX (RNAiMAX). After treatment (t=0), 200 cells were tracked for 6 hours, and their mean-squared displacement was measured as described elsewhere (Valencia et al., Oncotarget, 6:43438-43451 (2015)).

### Evaluation of Macrophage Gene Expression Via RT-qPCR

This experiment was performed as discussed in Example 8.

### Proliferation Study

Cells were plated at 5,000 cells per well in white 96-well plates and incubated for ˜24 hours. The next day the media were replaced and the LBL nanotubes were spiked in at various concentrations. The cells were then incubated for 48 hours before analyzing by CellTiter Glo 2.0 (Promega, Madison, WI) according to the manufacturer's instructions.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A nanotube comprising an anti-cancer agent, wherein said nanotube comprises nucleic acid (NA)-amphiphiles, each NA amphiphile comprising a hydrophilic NA headgroup and hydrophobic dialkyl tail comprising a hydrophobic spacer, wherein said anti-cancer agent is intercalated in said nanotube, and wherein said anti-cancer agent is selected from the group consisting of doxorubicin, gemcitabine, 5FU, carboplatin, cyclophosphamide, cisplatin, and oxaliplatin.

2. A nanotube comprising a hydrophobic therapeutic agent, wherein said nanotube comprises NA-amphiphiles, each NA amphiphile comprising a hydrophilic NA headgroup and hydrophobic dialkyl tail comprising a hydrophobic spacer, wherein said hydrophobic therapeutic agent is encapsulated in said nanotube, and wherein said hydrophobic therapeutic agent is a senotherapeutic agent.

3. (canceled)

4. A nanotube comprising an anti-cancer agent, wherein said nanotube comprises NA-amphiphiles, each NA amphiphile comprising a hydrophilic NA headgroup and hydrophobic dialkyl tail comprising a hydrophobic spacer, wherein said anti-cancer agent is encapsulated within said nanotube, and wherein said anti-cancer agent is selected from the group consisting of tamoxifen, paclitaxel, docetaxel, temozolomide, camptothecin, curcumin, dexamethasone, furosemide, IPI-549, and KPT-9274.

5. The nanotube of claim 1, wherein said hydrophilic NA headgroup comprises from about 4 nucleotides to about 52 nucleotides.

6. The nanotube of claim 1, wherein said hydrophilic NA headgroup comprises single stranded nucleic acid.

7. The nanotube of claim 1, wherein said hydrophilic NA headgroup comprises double stranded nucleic acid.

8. The nanotube of claim 1, wherein said hydrophilic NA headgroup comprises a non-targeting nucleotide sequence.

9. The nanotube of claim 8, wherein said non-targeting nucleotide sequence comprises a nucleotide sequence selected from the group consisting of CTCTTGGGGG (SEQ ID NO:1) and GGGGGTTCTC (SEQ ID NO:2).

10. The nanotube of claim 1, wherein said hydrophobic dialkyl tail comprising said hydrophobic spacer comprises a structure set forth in Formula I: 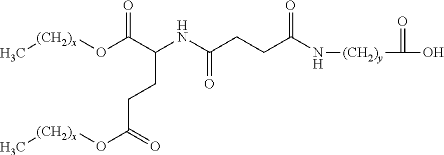
wherein x=15, and wherein y=11.

11. The nanotube of claim 1, wherein said NA-amphiphiles comprise a linker between said hydrophilic NA headgroup and said hydrophobic dialkyl tail.

12. The nanotube of claim 11, wherein said linker is selected from the group consisting of a near-infrared (NIR) light sensitive linker, a pH sensitive linker, a disulfide linker, an acetal, and a positively charged polypeptide.

13. A nanotube comprising NA-amphiphiles, wherein each NA-amphiphile comprises a hydrophilic NA headgroup and hydrophobic dialkyl tail comprising a hydrophobic spacer, wherein said hydrophilic NA headgroup comprises a microRNA (miRNA), a miRNA mimic, an anti-miRNA, or a small interfering RNA (siRNA).

14. The nanotube of claim 13, wherein said miRNA is selected from the group consisting of miR-34a, miR128, miR-21, miR-603, miR-218, miR-219, miR-183m, miR-451, miR-133, miR-134, miR-302c, miR-324, miR-379, miR-491, miR-340, miR-7, miR-128, miR-368-3p, miR-10b, miR-15a, miR-16, miR-17-5p, miR-26a, miR-29, miR-29b, miR-31, miR-33a, miR-34, miR-93, miR-101, miR-101-3p, miR-122, miR-122a, miR-125b, miR-130a, miR-133-b, miR-136, miR-143, miR-145, miR-146a-5p, miR-148a, miR-181d, miR-182, miR-183, miR-195, miR-199a-5p, microRNAs in the miR-200 family, miR-203, miR-203b-3p, miR-205, miR-206, miR-217, miR-298, miR-375, miR-490-3p, miR-539, miR-542-3p, miR-613, miR-638, miR-940, and a microRNA in the let-7 family.

15. (canceled)

16. The nanotube of claim 13, wherein said anti-mRNA is selected from the group consisting of anti-miR-9, anti-miR-10b, anti-miR-20a-5p, anti-miR-21, anti-miR-25-3p, anti-miR-26a, anti-miR-27a, anti-miR-29b, anti-miR-30b/30e/30d, anti-miR-31, anti-miR-34a, anti-miR-103, anti-miR-107, anti-miR-122, anti-miR-125b, anti-miR-126, anti-miR-139, anti-miR-143, anti-miR-146a, anti-miR-146b-5p, anti-miR-153, anti-miR-155, anti-miR-181a/b, anti-miR-182, anti-miR-199a-3p, anti-miR-199a-5p, anti-miR-200s, anti-miR-210, anti-miR-214, anti-miR-221, anti-miR-222, anti-miR-223, anti-miR-335, anti-miR-342, anti-miR-373, anti-miR-455-3p, anti-miR-429, anti-miR-493, anti-miR-494, anti-miR-520c, anti-miR-520h, and anti-miR-1908.

17. (canceled)

19. The nanotube of claim 13, wherein said NA-amphiphiles comprise a linker between said miRNA, said miRNA mimic, said anti-miRNA, or siRNA and said hydrophobic dialkyl tail.

20-22. (canceled)

23. The nanotube of claim 1, wherein said nanotube is coated, at least in part, by a layer comprising a polymer selected from the group consisting of polyethylenimine (PEI), poly(allylamine), a polyamine-based polymer, polylysine, polyarginine, polyglutamic acid, a polyamino ester, a polymethacrylate, a cyclodextrin-based polymer, fucoidan, chitosan, hyaluronic acid, dextran, dextran sulfate, β-cyclodextrin, cyclodextrins, alginic acid, alginate, cellulose sulfate, cellulose, heparin, protamine sulfate, carboxymethylcellulose, poly(styrene sulfonate), poly(dimethyldiallylammonium chloride), poly(N-isopropyl acrylamide), poly(acrylic acid), poly(methacrylic acid), poly(vinyl sulfate), poly(ethylene oxide), and poly(ethylene glycol).

24. The nanotube of claim 1, wherein said nanotube is coated, at least in part, by a layer comprising a non-targeting nucleotide sequence comprises a nucleotide sequence selected from the group consisting of CTCTTGGGGG (SEQ ID NO:1) and GGGGGTTCTC (SEQ ID NO:2).

25. The nanotube of claim 1, wherein said nanotube is coated, at least in part, by a layer comprising a polysaccharide selected from the group consisting of fucoidan, chitosan, hyaluronic acid, dextran, dextran sulfate, β-cyclodextrin, cyclodextrins, alginic acid, alginate, cellulose sulfate, cellulose, heparin, protamine sulfate, and carboxymethylcellulose.

26. The nanotube of claim 1, wherein said nanotube is coated, at least in part, by a layer comprising a miRNA selected from the group consisting of miR-34a, miR128, miR-21, miR-603, miR-218, miR-219, miR-183m, miR-451, miR-133, miR-134, miR-302c, miR-324, miR-379, miR-491, miR-340, miR-7, miR-128, and miR-368-3p, miR-10b, miR-15a, miR-16, miR-17-5p, miR-26a, miR-29, miR-29b, miR-31, miR-33a, miR-34, miR-93, miR-101, miR-101-3p, miR-122, miR-122a, miR-125b, miR-130a, miR-133-b, miR-136, miR-143, miR-145, miR-146a-5p, miR-148a, miR-181d, miR-182, miR-183, miR-195, miR-199a-5p, microRNAs in the miR-200 family, miR-203, miR-203b-3p, miR-205, miR-206, miR-217, miR-298, miR-375, miR-490-3p, miR-539, miR-542-3p, miR-613, miR-638, miR-940, and a microRNA in the let-7 family.

27. The nanotube of claim 1, wherein said nanotube is coated, at least in part, by a layer comprising an anti-miRNA selected from the group consisting of anti-miR-9, anti-miR-10b, anti-miR-20a-5p, anti-miR-21, anti-miR-25-3p, anti-miR-26a, anti-miR-27a, anti-miR-29b, anti-miR-30b/30e/30d, anti-miR-31, anti-miR-34a, anti-miR-103, anti-miR-107, anti-miR-122, anti-miR-125b, anti-miR-126, anti-miR-139, anti-miR-143, anti-miR-146a, anti-miR-146b-5p, anti-miR-153, anti-miR-155, anti-miR-181a/b, anti-miR-182, anti-miR-199a-3p, anti-miR-199a-5p, anti-miR-200s, anti-miR-210, anti-miR-214, anti-miR-221, anti-miR-222, anti-miR-223, anti-miR-335, anti-miR-342, anti-miR-373, anti-miR-455-3p, anti-miR-429, anti-miR-493, anti-miR-494, anti-miR-520c, anti-miR-520h, and anti-miR-1908.

28. A method for treating a mammal having cancer, wherein said method comprises:
administering a composition comprising the nanotube of claim 1 to said mammal.

29. The method of claim 28, wherein said mammal is a human.

30. The method of claim 28, wherein said cancer is selected from the group consisting of a glioblastoma, an astrocytoma, an oligodendroglioma, an oligoastrocytoma, an ependymoma, a medulloblastoma, a meningioma, a diffuse intrinsic pontine glioma (DIPG), a breast cancer, a colon cancer, a liver cancer, a pancreatic cancer, a prostate cancer, a lung cancer, an ovarian cancer, a kidney cancer, a spleen cancer, and a gastric cancer.

31. A method for repolarizing a tumor-associated microglia and macrophage (TAM) to an M1-phenotype within a mammal having cancer, wherein said method comprises:
administering a composition comprising the nanotube of claim 1 to said mammal.

32. (canceled)

33. (canceled)
